(19) United States
(12) Patent Application Publication
(10) Pub. No.: US 2024/0360062 A1
(43) Pub. Date: Oct. 31, 2024

(54) HALOGENATED ETHER-CONTAINING ELECTROLYTES

(71) Applicant: Feon Energy, Inc., Woburn, MA (US)

(72) Inventors: Zhiao YU, Cambridge, MA (US); Wenxiao HUANG, Medford, MA (US)

(21) Appl. No.: 18/644,624

(22) Filed: Apr. 24, 2024

Related U.S. Application Data
(60) Provisional application No. 63/461,496, filed on Apr. 24, 2023.

(51) Int. Cl. C07C 43/12; C07C 41/16; C07C 41/50; C07C 41/60; C07C 43/313; C07C 43/32; C07D 307/10; C07D 317/16; H01M 10/0525; H01M 10/0568; H01M 10/0569; H01M 10/42

(52) U.S. Cl. CPC C07C 43/123; C07C 41/16; C07C 41/50; C07C 41/60; C07C 43/313; C07C 43/32; C07D 307/10; C07D 317/16; H01M 10/0525; H01M 10/0568; H01M 10/0569; H01M 10/4235; H01M 2300/0034; H01M 2300/0037

(57) ABSTRACT

Provided herein are halogenated ether compounds of Formula (I), Formula (II), or Formula (III):

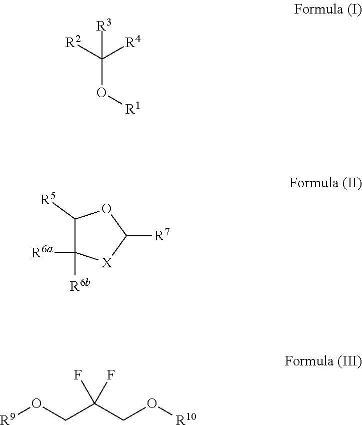

Also provided are electrolytes comprising one or more compounds of Formula (I), Formula (II), or Formula (III) and electrochemical cells comprising electrolytes comprising one or more compounds of Formula (I), Formula (II), or Formula (III).

### CROSS-REFERENCE TO RELATED APPLICATIONS

This application claims the priority benefit of U.S. Provisional Application No. 63/461,496, filed Apr. 24, 2023, the entire contents of which are hereby incorporated by reference herein.

### FIELD

The present disclosure relates generally to halogenated ether compounds, and more specifically to halogenated ether compounds and use thereof in electrolytes and electrochemical cells.

### BACKGROUND

Existing electrolyte formulations used in commercial lithium ion batteries are incompatible with lithium metal anodes due to the resulting low coulombic efficiency and the formation of lithium dendrites during battery cycling. The design of commercially viable electrolyte compounds that are simultaneously compatible with both lithium metal anodes (or graphite, graphite-silicon composite, graphite-silicon monoxide composite, silicon monoxide, and silicon anodes) and high-voltage cathodes is lacking. Accordingly, there is a need for suitable electrolytes to enable the preparation of high-performance lithium batteries.

### BRIEF SUMMARY

Provided herein are halogenated ether compounds that exhibit excellent properties for as electrolytes, or electrolyte components in electrochemical cell, including, for example, batteries.

A major challenge encountered in the design of lithium ion batteries is the potential for electrolyte components to undergo undesired side reactions at the anode. Such parasitic reactions can result in low Coulombic efficiency (CE) for the battery, as well as continuous loss of the active lithium reservoir and consumption of the electrolyte. To prevent such side reactions from occurring, it is beneficial to form a protective solid electrolyte interphase (SEI) that can inhibit reaction between the anode and the electrolyte components. Additionally, the electrolyte components used in batteries (including, for example, lithium ion batteries) should have both high oxidation potentials and low reduction potentials, in order to both avoid undergoing reduction at the anode and avoid undergoing oxidation at the cathode. While ether-based electrolytes can suppress the formation of undesirable morphologies at lithium anodes, they are prone to oxidation at high-voltage cathodes. In contrast, the halogenated ether compounds provided herein exhibit greater stability against oxidation than the corresponding ethers, while still maintaining the stability at lithium anodes. Additionally, the halogenated ether compounds provided herein may facilitate the formation of more robust SEI layers than do other electrolyte compounds. The presence of fluorine functional groups at selected positions on the halogenated ether compounds provided herein are beneficial to the stability and function of these compounds for three main reasons: (1) fluorine functional groups can weaken the solvation ability of the solvent and provide stronger Li-ion/anion interaction in the electrolyte, leading to more anion-derived SEI, (2) the fluorine present in these compounds can provide an additional fluorine source that can be incorporated into the SEI, and (3) the strong electron-withdrawing ability of the fluorine functional groups can stabilize the ethereal oxygen found in these compounds, thus preventing it from undergoing oxidation at high-voltage cathodes. Additionally, the placement of fluorine functional groups should be rationally designed. While rationally selected fluorination can achieve balanced beneficial effects, excessive fluorination may result in sluggish transport or insolubility of salts in these electrolytes. In one aspect, provided herein is a compound of Formula (I), Formula (II), or Formula (III):

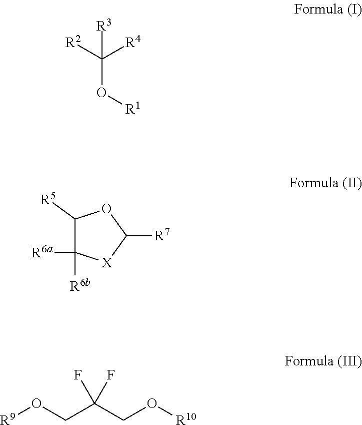

wherein:
X is selected from the group consisting of *—(CH₂)ₙO—**, *—(CH₂)ₙ—**, and *—O(CHR⁸)—**, wherein * denotes the point of attachment to the carbon bearing R^{6a} and R^{6b}, and ** denotes the point of attachment to the carbon bearing R⁷;
n is 0-3;
R¹ is C₁-C₄ alkyl substituted by one or more halogen, wherein each halogen is independently selected;
R² is selected from the group consisting of R^{2′} and —OR^{2′}, wherein R^{2′} is C₁-C₄ alkyl substituted by one or more halogen, wherein each halogen is independently selected;
R³ is selected from the group consisting of H, R^{3′}, and —OR^{3′}, wherein R^{3′} is C₁-C₄ alkyl substituted by one or more halogen, wherein each halogen is independently selected;
R⁴ is selected from the group consisting of H, R^{4′}, and —OR^{4′}, wherein R^{4′} is C₁-C₄ alkyl substituted by one or more halogen, wherein each halogen is independently selected;
R⁵ is selected from the group consisting of H, R^{5′}, and —CH₂OR^{5′}, wherein R^{5′} is C₁-C₄ alkyl substituted by one or more halogen, wherein each halogen is independently selected;
R^{6a} is selected from the group consisting of H, halogen, R^{6′}, and —CH₂OR^{6′}, wherein R^{6′} is C₁-C₄ alkyl substituted by one or more halogen, wherein each halogen is independently selected;
R^{6b} is selected from the group consisting of H and halogen;
R⁷ is selected from the group consisting of H, R^{7′}, and —CH₂OR^{7′}, wherein R^{7′} is C₁-C₄ alkyl substituted by one or more halogen, wherein each halogen is independently selected; and
R⁸ is selected from the group consisting of H, R^{8′}, and —CH₂OR^{8′}, wherein R^{8′} is C₁-C₄ alkyl substituted by one or more halogen, wherein each halogen is independently selected;
R⁹ is C₁-C₄ alkyl;
R¹⁰ is C₁-C₄ alkyl;
provided that when R⁷ is H, R^{6a} is H, and R^{6b} is H, then R⁵ is R^{5′}, and provided that the compound is not

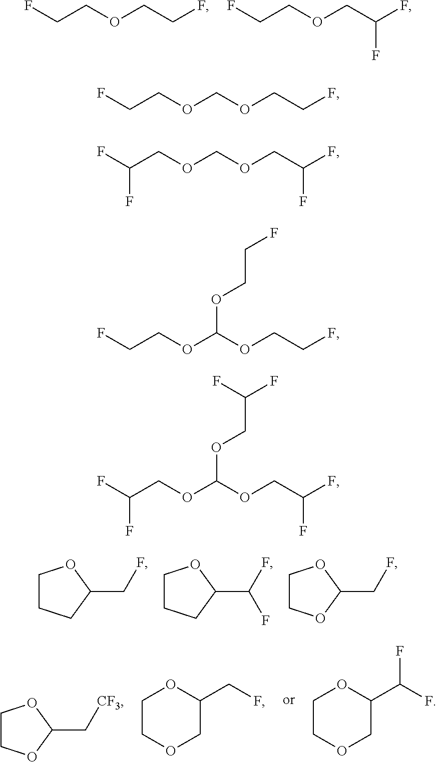

In some variations, the compound of Formula (I) is a compound of Formula (I-A).

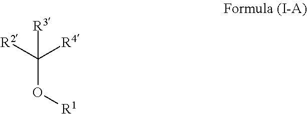

In some variations, the compound of Formula (I) is a compound of Formula (I-B).

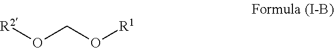

In some variations, the compound of Formula (I) is a compound of Formula (I-C).

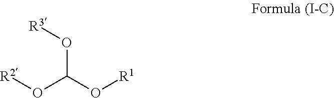

In some variations, the compound of Formula (I) is a compound of Formula (I-D).

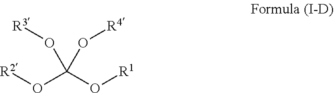

In some variations, the compound of Formula (II) is a compound of Formula (II-A).

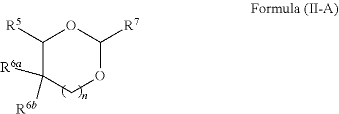

In some variations, the compound of Formula (II) is a compound of Formula (II-B).

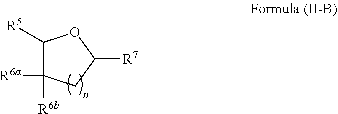

In some variations, the compound of Formula (II) is a compound of Formula (II-C).

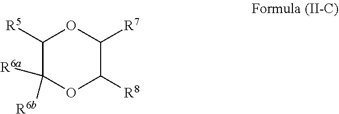

In one aspect, provided is a compound selected from the group consisting of

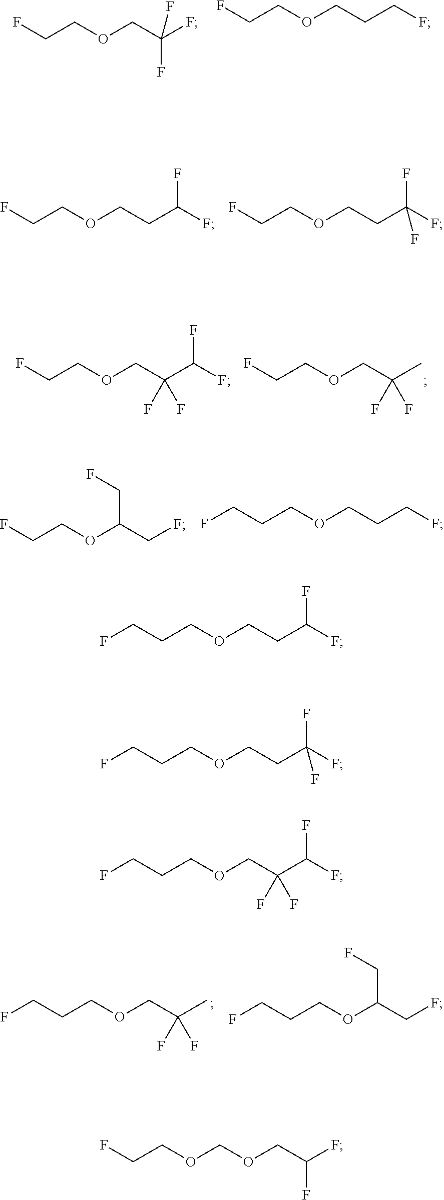

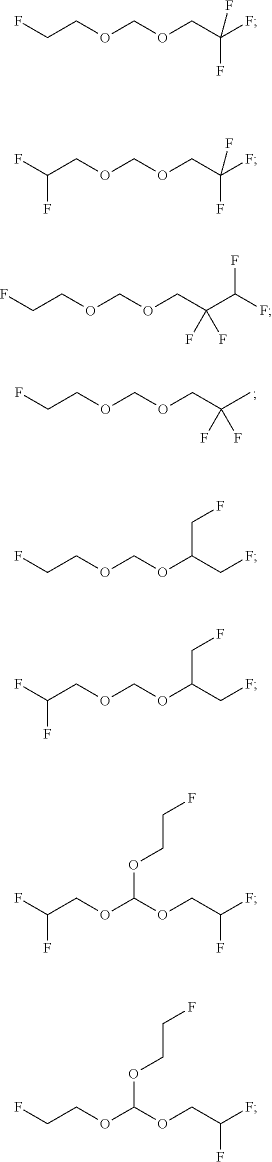

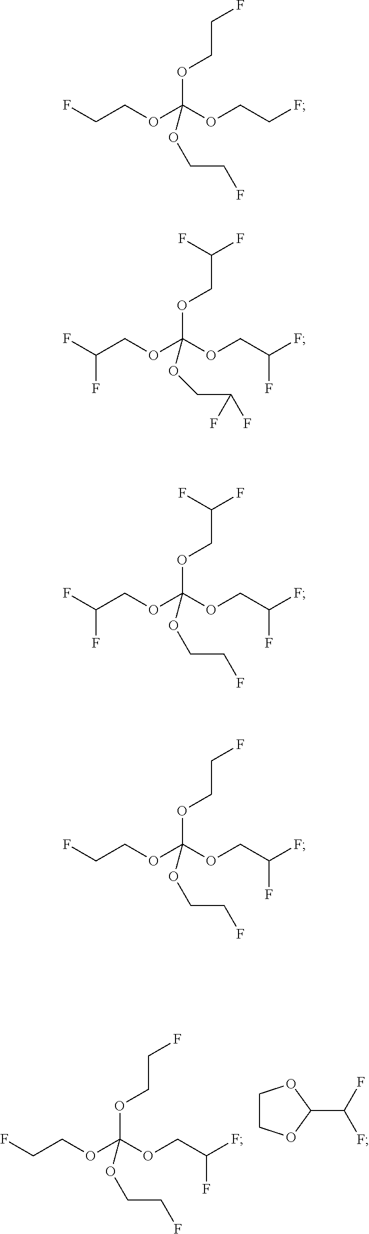

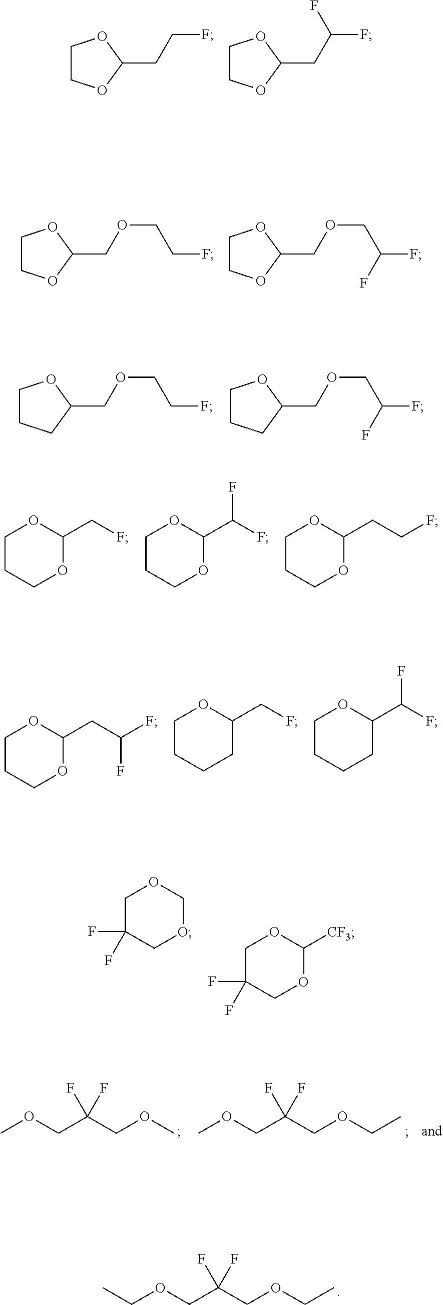

In one aspect, provided herein is a method of preparing a compound of Formula (I-A), comprising reacting a compound of Formula (S1)

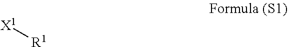

with a compound of Formula (S2)

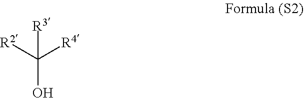

and a base, to provide a compound of Formula (I-A), wherein X¹ is selected from the group consisting of

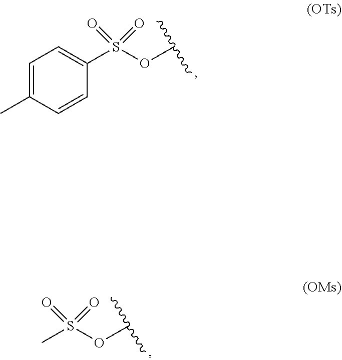

### Cl, Br, and I.

In one aspect, provided herein is a method of preparing a compound of Formula (I-B), comprising reacting a compound of Formula (S3)

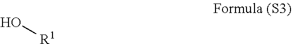

and a compound of formula (S4)

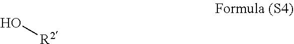

with a compound of Formula (S5)

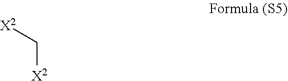

and a base, to provide the compound of Formula (I-B), wherein each X² is independently selected from the group consisting of Cl, Br, and I.

In one aspect, provided herein is a method of preparing a compound of Formula (I-C), comprising reacting a compound of Formula (S3)

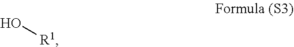

a compound of Formula (S4)

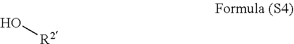

a compound of Formula (S6)

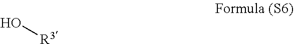

with a compound of Formula (S7)

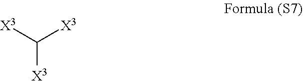

and a base, to provide the compound of Formula (I-C), wherein each X³ is independently selected from the group consisting of Cl, Br, and I.

In one aspect, provided herein is a method of preparing a compound of Formula (I-D), comprising reacting a compound of Formula (S3)

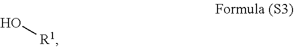

a compound of Formula (S4)

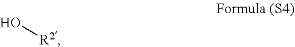

a compound of Formula (S6)

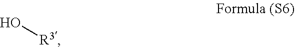

a compound of Formula (S8)

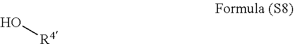

with a compound of Formula (S9)

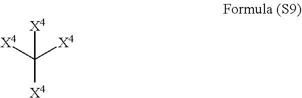

and a base, to provide the compound of Formula (I-D), wherein each X⁴ is independently selected from the group consisting of Cl, Br, and I.

In one aspect, provided herein is a method of preparing a compound of Formula (III), comprising reducing a compound of Formula (S10)

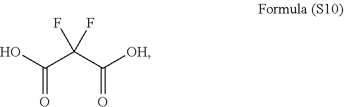

to provide a compound of Formula (S11)

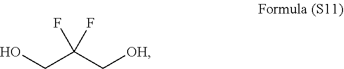

and reacting the compound of Formula (S11) with a compound of Formula (S12)

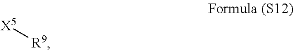

a compound of Formula (S13)

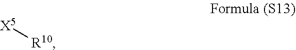

to provide the compound of Formula (III), wherein each X⁵ is independently selected form the group consisting of

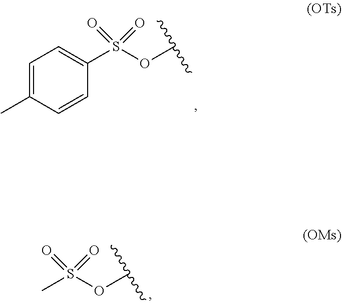

### Cl, Br, and I.

In another aspect, provided herein is an electrolyte comprising a first halogenated ether component of Formula (I), Formula (II), or Formula (III):

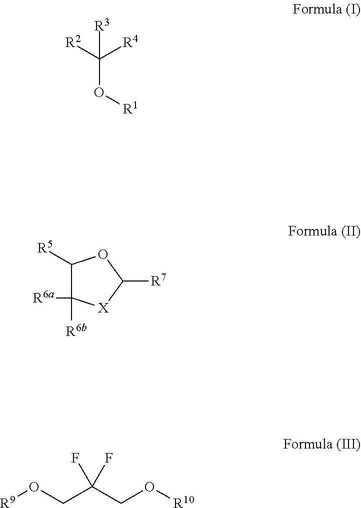

wherein:
X is selected from the group consisting of *—(CH₂)ₙO—**, *—(CH₂)ₙ—**, and *—O(CHR⁸)—**, wherein * denotes the point of attachment to the carbon bearing R^{6a} and R^{6b}, and ** denotes the point of attachment to the carbon bearing R⁷;
n is 0-3;
R¹ is C₁-C₄ alkyl substituted by one or more halogen, wherein each halogen is independently selected;
R² is selected from the group consisting of R^{2′} and —OR^{2′}, wherein R^{2′} is C₁-C₄ alkyl substituted by one or more halogen, wherein each halogen is independently selected;
R³ is selected from the group consisting of H, R^{3′}, and —OR^{3′}, wherein R^{3′} is C₁-C₄ alkyl substituted by one or more halogen, wherein each halogen is independently selected;
R⁴ is selected from the group consisting of H, R^{4′}, and —OR^{4′}, wherein R^{4′} is C₁-C₄ alkyl substituted by one or more halogen, wherein each halogen is independently selected;
R⁵ is selected from the group consisting of H, R^{5′}, and —CH₂OR^{5′}, wherein R^{5′} is C₁-C₄ alkyl substituted by one or more halogen, wherein each halogen is independently selected;
R^{6a} is selected from the group consisting of H, halogen, R^{6′}, and —CH₂OR^{6′}, wherein R^{6′} is C₁-C₄ alkyl substituted by one or more halogen, wherein each halogen is independently selected;
R^{6b} is selected from the group consisting of H and halogen;
R⁷ is selected from the group consisting of H, R^{7′}, and —CH₂OR^{7′}, wherein R^{7′} is C₁-C₄ alkyl substituted by one or more halogen, wherein each halogen is independently selected;
R⁸ is selected from the group consisting of H, R^{8′}, and —CH₂OR^{8′}, wherein R^{8′} is C₁-C₄ alkyl substituted by one or more halogen, wherein each halogen is independently selected; and
R⁹ is C₁-C₄ alkyl;
R¹⁰ is C₁-C₄ alkyl,
provided that when R⁷ is H, R^{6a} is H, and R^{6b} is H, then R⁵ is R^{5′}, and
provided that the compound is not or

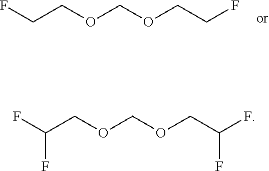 [SMILES: FCCOCOCCF ; FC(F)COCOCC(F)F]

In some embodiments, the first halogenated ether component is a compound selected from the group consisting of

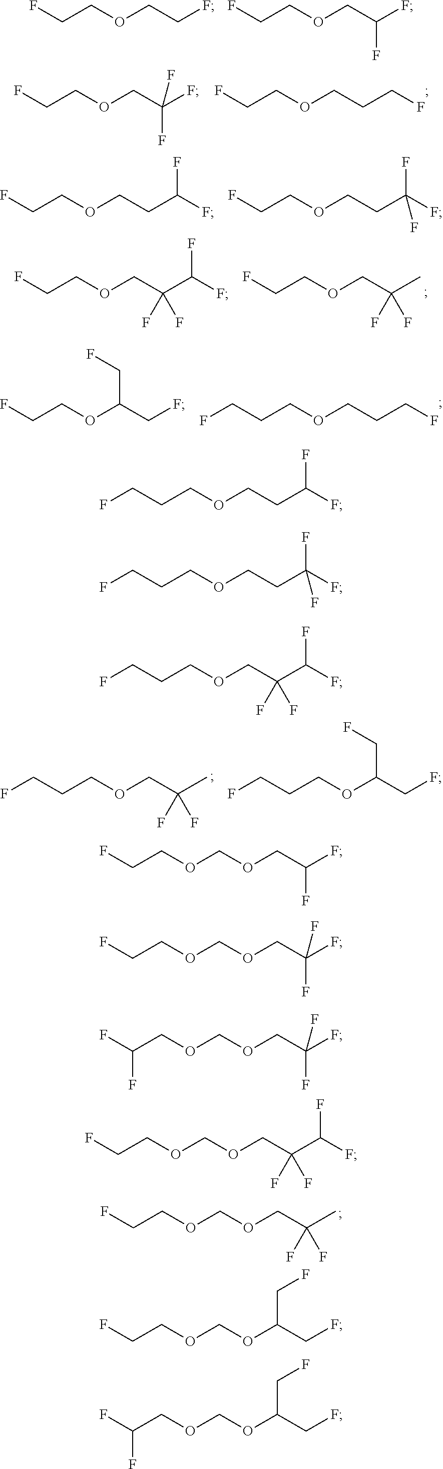

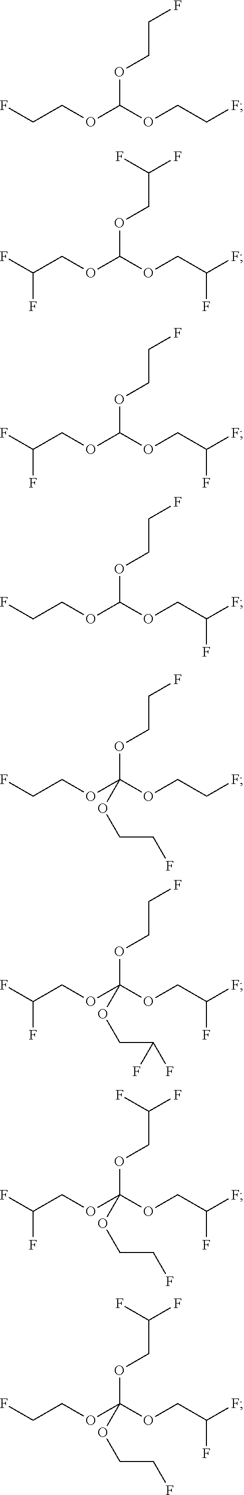

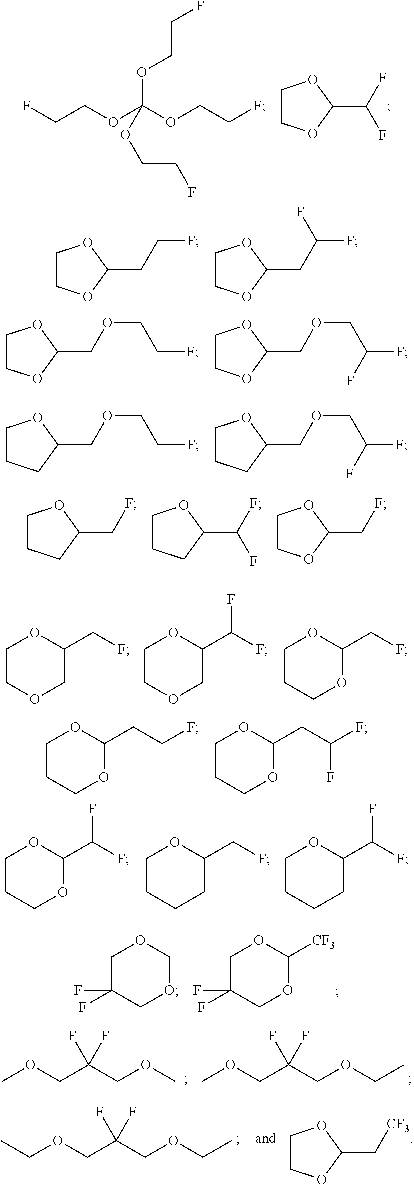

In some embodiments, the electrolyte comprises one or more additional halogenated ether components, each of which is independently a compound of Formula (I), Formula (II), or Formula (III). In some embodiments, the electrolyte comprises a secondary component, wherein the secondary component is a solvent that is not a compound of Formula (I), Formula (II), or Formula (III). In some embodiments, the second component is selected from the group consisting of ethylene carbonate (EC); propylene carbonate (PC); dimethyl carbonate (DMC); diethyl carbonate (DEC); ethyl methyl carbonate (EMC); vinyl carbonate (VC); vinyl ethylene carbonate (VEC); fluoroethylene carbonate (FEC); difluoroethylene carbonate (DFEC); 3,3,3-trifluoropropylene carbonate (TFPC); monofluoroethyl methyl carbonate (F1EMC); difluoroethyl methyl carbonate (F2EMC); trifluoroethyl methyl carbonate (F3EMC); bis(2,2,2-trifluoroethyl) carbonate (TFEC); succinic anhydride (SA), butyric anhydride (BA); 1,2-dimethyoxylethane (DME); 1,3-dioxolane (DOL); 1,4-dioxane (DOX); tetrahydrofuran (THF); tetravinyl silane (TVSI); acetonitrile (AN); ethyl acetate (EA); methyl acetate (MA); methyl propanoate (MP); succinonitrile (SN); adiponitrile (ADN); 1,3,6-Hexanetricarbonitrile (HTCN); trimethyl borate (TMB); triphenyl borate (TPB); triethyl borate (TEB); tris(pentafluorophenyl)borane (TPFPB); tris(trimethylsilyl)phosphate (TTSB); tris(2,2,2-trifluoroethyl) borate (TTFEB); trimethyl phosphate (TMP); triethyl phosphate (TEP); tris(trimethylsilyl)phosphate (TTSP); tris(trimethylsilyl)phosphite (TTSPi); tris(2,2,2-trifluoroethyl) phosphate (TFEPa); tris(2,2,2-trifluoroethyl) phosphite (TFEPi); (pentafluorophenyl)diphenyl phosphine (PFPDPP); tris(pentafluorophenyl) phosphine (TPFPP); 1,3,2-dioxathiolane-2,2-dioxide (DTD); 1,3-propanesultone (PS); prop-1-ene-1,3-sultone (PES); propanediol cyclic sulfate (PCS); ethylene sulfite (ES); 1,4-butane sultone (BS); dimethyl sulfoxide (DMSO); methylene methanedisulfonate (MMDS); N,N-Dimethylformamide (DMF); gamma-butyrolactone (BL); bis(2,2,2-trifluoroethyl) ether (BTFE); 1,1,2,2-tetrafluoroethyl-2,2,3,3-tetrafluoropropyl ether (TTE); 1H,1H,5H-octafluoropentyl-1,1,2,2-tetrafluoroethylether (OTE); 2,2,2-trifluoroethyl 1,1,2,2-tetrafluoroethyl ether (HFE); tris(2,2,2-trifluoroethyl) orthoformate (TFEO); 1,2-bis(1,1,2,2-tetrafluoroethoxy)ethane (F8DEE); 1,1,1,2,3,3-hexafluoro-3-(2,2,2-trifluoroethoxy)propane; hexafluoroisopropyl methyl ether; 1-fluoro-2-(2-methoxyethoxy)ethane; 1,1-difluoro-2-(2-methoxyethoxy)ethane; 1,1,1-trifluoro-2-(2-methoxyethoxy)ethane; 1-ethoxy-2-(2-fluoroethoxy)ethane; 2-(2-ethoxyethoxy)-1,1-difluoroethane; 1,2-bis(2-fluoroethoxy)ethane; 1,1-difluoro-2-(2-(2-fluoroethoxy)ethoxy)ethane; 1,1,1-trifluoro-2-(2-(2-fluoroethoxy)ethoxy)ethane; 2-(2-ethoxyethoxy)-1,1,1-trifluoroethane; 1,2-bis(2,2-difluoroethoxy)ethane; 2-(2-(2,2-difluoroethoxy)ethoxy)-1,1,1-trifluoroethane; or 1,2-bis(2,2,2-trifluoroethoxy)ethane; 2-fluoro-1,3-dimethoxypropane; 2-fluoro-1,3-diethoxypropane; 2,2-difluoro-1,3-dimethoxypropane; or 2,2-difluoro-1,3-diethoxypropane; 2-fluoro-1,3-bis(2-fluoroethoxy)propane; 2-fluoro-1,3-bis(2,2-difluoroethoxy)propane; 2-fluoro-1,3-bis(2,2,2-trifluoroethoxy)propane; 2,2-difluoro-1,3-bis(2-fluoroethoxy)propane; 2,2-difluoro-1,3-bis(2,2-difluoroethoxy)propane; 2,2-difluoro-1,3-bis(2,2,2-trifluoroethoxy)propane; and mixtures of any of the foregoing. In some embodiments, the amount of the secondary component in the electrolyte is between about 0.5 wt. % and about 99.0 wt. %.

In some embodiments, the electrolyte comprises one or more salts. In some embodiments, the salt is selected from the group consisting of a lithium salt, a potassium salt, a sodium salt, a cesium salt, a magnesium salt, a zinc salt, a calcium salt, a silver salt, an aluminum salt, a lanthanum salt, and mixtures of any of the foregoing. In some embodiments, the salt is selected from the group consisting of lithium bis(fluorosulfonyl)imide (LiFSI), lithium bis(trifluoromethanesulfonyl)imide (LiTFSI), lithium bis(pentafluoroethanesulfonyl)imide (LiBETI), lithium hexafluorophosphate (LiPF₆), lithium hexafluoroarsenate (LiAsF₆), lithium tetrafluoroborate (LiBF₄), lithium bis(oxalato)borate (LiBOB), lithium difluoro(oxalato)borate (LiDFOB), lithium difluorophosphate (LiDFP), lithium difluoro(dioxalato)phosphate (LiDFDOP), lithium tetrafluoro(oxalato)phosphate (LiTFOP), lithium nitrate (LiNO₃), lithium perchlorate (LiClO₄), lithium triflate (LiTf), lithium trifluoroacetate (LiTFA), lithium 4,5-dicyano-2-(trifluoromethyl)imidazole (LiTDI), sodium hexafluorophosphate (NaPF₆), sodium bis(fluorosulfonyl)imide (NaFSI), sodium bis(trifluoromethanesulfonyl)imide (NaTFSI), sodium triflate (NaTf), potassium hexafluorophosphate (KPF₆), potassium bis(fluorosulfonyl)imide (KFSI), potassium bis(trifluoromethanesulfonyl)imide (KTFSI), potassium triflate (KTf), cesium bis(fluorosulfonyl)imide (CsFSI), cesium bis(trifluoromethanesulfonyl)imide (CsTFSI), magnesium bis(trifluoromethanesulfonyl)imide (Mg(TFSI)₂), zinc bis(trifluoromethanesulfonyl)imide (Zn(TFSI)₂), calcium bis(trifluoromethanesulfonyl)imide (Ca(TFSI)₂), silver bis(trifluoromethanesulfonyl)imide (AgTFSI), aluminum bis(trifluoromethanesulfonyl)imide (Al(TFSI)₃), lanthanum bis(trifluoromethanesulfonyl)imide (La(TFSI)₃), and mixtures of any of the foregoing.

In another aspect, provided herein is an electrochemical cell comprising an anode, a cathode, and any of the electrolytes described herein. In some embodiments, the electrochemical cell is a battery. In some embodiments, the anode comprises an element selected from the group consisting of lithium, sodium, and potassium. In some embodiments, the anode comprises lithium metal. In some embodiments, the anode comprises a surface protection layer comprising fluorine. In some embodiments, the anode comprises the anode comprises a material selected from the group consisting of lithium metal, graphite, silicon, silicon oxide (SiOₓ), graphite/silicon composite, graphite/silicon oxide (SiOₓ) composite, graphite/silicon nitride (Si₃N₄) composite, graphite/silicon carbide (SiC) composite, sodium metal, hard carbon, potassium metal, and mixtures of any of the foregoing. In some embodiments, the cathode comprises sulfur, a lithium nickel manganese cobalt oxide, a lithium nickel cobalt aluminum oxide (NCA), a lithium nickel manganese aluminum oxide (NMA), a lithium nickel manganese cobalt aluminum oxide (NMCA), a lithium nickel oxide (LNO), a lithium nickel manganese oxide (LiNi_{0.5}Mn_{1.5}O₄), a lithium cobalt oxide (LCO), a lithium manganese oxide (LMO), a lithium and manganese rich cathode (LMR or LLMO), a lithium iron phosphate (LFP), a lithium cobalt phosphate (LCP), a lithium manganese phosphate (LMP), a lithium manganese iron phosphate (LMFP), a transition metal sulfide, a sodium vanadium phosphate (Na₃V₂(PO₄)₃), a sodium copper nickel iron manganese oxide (Na[Cu_{1/9}Ni_{2/9}Fe_{1/3}Mn_{1/3}]O₂), a Prussian white (R—Na_{1.92}Fe[Fe(CN)₆]), and mixtures of any of the foregoing.

### DESCRIPTION OF THE FIGURES

The present application can be understood by reference to the following description taken in conjunction with the accompanying figures.

FIG. 1 depicts the voltage profile for an anode-free battery using a 4 M solution of lithium bis(fluorosulfonyl)imide (LiFSI) in Compound 39 as an electrolyte.

FIG. 2 depicts the capacity vs. voltage curve for the 5^{th} cycle for an anode-free battery using a 4 M solution of lithium bis(fluorosulfonyl)imide (LiFSI) in Compound 39 as an electrolyte.

FIG. 3 depicts the voltage profile for an anode-free battery using a 3 M solution of LiFSI in Compound 41 as an electrolyte.

FIG. 4 depicts the capacity vs. voltage curve for the 5^{th} cycle for an anode-free battery using a 3 M solution of LiFSI in Compound 41 as an electrolyte.

FIG. 5 depicts the capacity vs. voltage curve for the 5^{th} cycle for an anode-free battery using a 2 M solution of LiFSI in Compound 52 as an electrolyte.

FIG. 6 depicts the cycling profile of a lithium metal battery using a 2 M solution of LiFSI in Compound 52 as an electrolyte.

FIG. 7 depicts the voltage profile for an anode-free battery using a 2 M solution of LiFSI in Compound 55 as an electrolyte.

FIG. 8 depicts the capacity vs. voltage curve for the 5^{th} cycle for an anode-free battery using a 2 M solution of LiFSI in Compound 55 as an electrolyte.

FIG. 9 depicts the cycling profile of a lithium metal battery using a 2 M solution of LiFSI in Compound 1 as an electrolyte.

FIG. 10 depicts the cycling profile of a lithium metal battery using a 2 M solution of LiFSI in Compound 2 as an electrolyte.

FIG. 11 depicts the cycling profile of a lithium metal battery using a solution of 2 M LiFSI and 0.1M LiTFSI dissolved in a binary solution of Compound 2 and 2-(2-(2,2-difluoroethoxy)ethoxy)-1,1,1-trifluoroethane (F5) (1:5 v/v, respectively) as an electrolyte.

FIG. 12 depicts the cycling profile at 45° C. (line extending beyond 600 cycles) and 60° C. (line ending before 300 cycles) of two lithium metal batteries using a solution of 2 M LiFSI and 0.1M LiTFSI dissolved in a binary solution of Compound 2 and 2-(2-(2,2-difluoroethoxy)ethoxy)-1,1,1-trifluoroethane (F5) (1:5 v/v, respectively) as an electrolyte.

FIG. 13 depicts the cycling profiles of four anode-free batteries, each using a solution of 3 M LiFSI in a mixture of Compound 2, F5, and 1,1,2,2-tetrafluoroethyl-2,2,3,3-tetrafluoropropyl ether (TTE).

FIG. 14 depicts the capacity vs. voltage curve for the 5^{th} cycle for an anode-free battery using a 2 M solution of LiFSI in Compound 23 as an electrolyte.

FIG. 15 depicts the cycling profile of an anode-free battery using a 3 M solution of LiFSI in a commercial non-fluorinated ether solvent, 1,2-dimethoxyethane (DME), as an electrolyte, which serves as a control.

FIG. 16 depicts a ¹H-NMR spectrum of Compound 1.

FIG. 17 depicts a GC-MS spectrum of Compound 1.

FIG. 18 depicts a ¹H-NMR spectrum of Compound 2.

FIG. 19 depicts a GC-MS spectrum of Compound 2.

FIG. 20 depicts a ¹H-NMR spectrum of Compound 10.

FIG. 21 depicts a GC-MS spectrum of Compound 10.

FIG. 22 depicts a ¹H-NMR spectrum of Compound 23.

FIG. 23 depicts a GC-MS spectrum of Compound 23.

FIG. 24 depicts a ¹H-NMR spectrum of 1,1-Bis(2-fluoroethoxy)methane.

FIG. 25 depicts a GC-MS spectrum of 1,1-Bis(2-fluoroethoxy)methane.

FIG. 26 depicts a ¹H-NMR spectrum of 1,1-Bis(2,2-difluoroethoxy)methane.

FIG. 27 depicts a GC-MS spectrum of 1,1-Bis(2,2-difluoroethoxy)methane.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### Definitions

Reference to “about” a value or parameter herein includes (and describes) embodiments that are directed to that value or parameter per se. For example, description referring to “about X” includes description of “X”. In some embodiments, the term “about” when used in association with a measurement, or used to modify a value, a unit, a constant, or a range of values, refers to variations of ±10%, ±5%, or ±2%.

Reference to “between” two values or parameters herein includes (and describes) embodiments that include those two values or parameters per se. For example, description referring to “between x and y” includes description of “x” and “y” per se.

It is understood that aspects and variations described herein also include “consisting” and/or “consisting essentially of” aspects and variations.

“Alkyl” as used herein refers to and includes, unless otherwise stated, a saturated linear (i.e., unbranched) or branched univalent hydrocarbon chain or combination thereof, having the number of carbon atoms designated (i.e., C₁-C₄ means one to four carbon atoms). Examples of alkyl groups include, but are not limited to, groups such as methyl, ethyl, n-propyl, isopropyl, n-butyl, t-butyl, isobutyl, sec-butyl, and the like.

“Alkylene” as used herein refers to the same residues as alkyl, but having bivalency. Examples of alkylene include, but are not limited to, groups such as methylene (—CH₂—), ethylene (—CH₂CH₂—), propylene (—CH₂CH₂CH₂—), and the like.

“Halogen” or “Halo” refers to elements of the Group 17 series having atomic number 9 to 85. Preferred halo groups include the radicals of fluorine, chlorine, bromine and iodine.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

It is understood that an optionally substituted moiety can be substituted with more than five substituents, if permitted by the number of valences available for substitution on the moiety. For example, a propyl group can be substituted with seven halogen atoms to provide a perhalopropyl group. The substituents may be the same or different.

### Compounds

In one aspect, provided herein is a compound of Formula (I), Formula (II), or Formula (III):

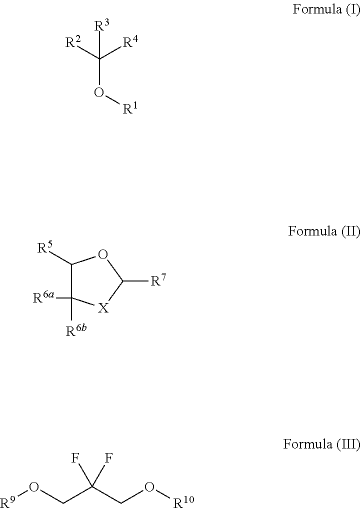

wherein:
X is selected from the group consisting of *—(CH₂)ₙO—**, *—(CH₂)ₙ—**, and *—O(CHR⁸)—**, wherein * denotes the point of attachment to the carbon bearing R^{6a} and R^{6b}, and ** denotes the point of attachment to the carbon bearing R⁷;
n is 0-3;
R¹ is C₁-C₄ alkyl substituted by one or more halogen, wherein each halogen is independently selected;
R² is selected from the group consisting of R^{2′} and —OR^{2′}, wherein R^{2′} is C₁-C₄ alkyl substituted by one or more halogen, wherein each halogen is independently selected;
R³ is selected from the group consisting of H, R^{3′}, and —OR^{3′}, wherein R^{3′} is C₁-C₄ alkyl substituted by one or more halogen, wherein each halogen is independently selected;
R⁴ is selected from the group consisting of H, R^{4′}, and —OR^{4′}, wherein R^{4′} is C₁-C₄ alkyl substituted by one or more halogen, wherein each halogen is independently selected;
R⁵ is selected from the group consisting of H, R^{5′}, and —CH₂OR^{5′}, wherein R^{5′} is C₁-C₄ alkyl substituted by one or more halogen, wherein each halogen is independently selected;
R^{6a} is selected from the group consisting of H, halogen, R^{6′}, and —CH₂OR^{6′}, wherein R^{6′} is C₁-C₄ alkyl substituted by one or more halogen, wherein each halogen is independently selected;
R^{6b} is selected from the group consisting of H and halogen;
R⁷ is selected from the group consisting of H, R^{7′}, and —CH₂OR^{7′}, wherein R^{7′} is C₁-C₄ alkyl substituted by one or more halogen, wherein each halogen is independently selected;
R⁸ is selected from the group consisting of H, R^{8′}, and —CH₂OR^{8′}, wherein R^{8′} is C₁-C₄ alkyl substituted by one or more halogen, wherein each halogen is independently selected;
R⁹ is C₁-C₄ alkyl; and
R¹⁰ is C₁-C₄ alkyl,
provided that when R⁷ is H, R^{6a} is H, and R^{6b} is H, then R⁵ is R^{5′}, and
provided that the compound is not

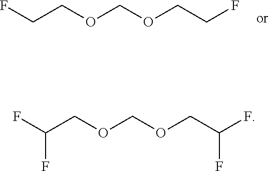 [SMILES: FCCOCOCCF ; FC(F)COCOCC(F)F]

In one aspect, provided herein is a compound of Formula (I), Formula (II), or Formula (III):

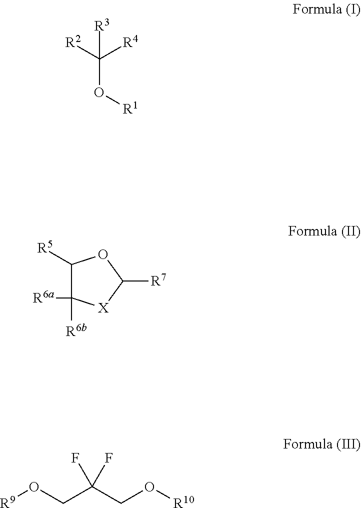

wherein:
X is selected from the group consisting of *—(CH₂)ₙO—**, *—(CH₂)ₙ—**, and *—O(CHR⁸)—**, wherein * denotes the point of attachment to the carbon bearing R^{6a} and R^{6b}, and ** denotes the point of attachment to the carbon bearing R⁷;
n is 0-3;
R¹ is C₁-C₄ alkyl substituted by one or more halogen, wherein each halogen is independently selected;
R² is selected from the group consisting of R^{2′} and —OR^{2′}, wherein R^{2′} is C₁-C₄ alkyl substituted by one or more halogen, wherein each halogen is independently selected;
R³ is selected from the group consisting of H, R^{3′}, and —OR^{3′}, wherein R^{3′} is C₁-C₄ alkyl substituted by one or more halogen, wherein each halogen is independently selected;
R⁴ is selected from the group consisting of H, R^{4′}, and —OR^{4′}, wherein R^{4′} is C₁-C₄ alkyl substituted by one or more halogen, wherein each halogen is independently selected;
R⁵ is selected from the group consisting of H, R^{5′}, and —CH₂OR^{5′}, wherein R^{5′} is C₁-C₄ alkyl substituted by one or more halogen, wherein each halogen is independently selected;
R^{6a} is selected from the group consisting of H, halogen, R^{6′}, and —CH₂OR^{6′}, wherein R^{6′} is C₁-C₄ alkyl substituted by one or more halogen, wherein each halogen is independently selected;
R^{6b} is selected from the group consisting of H and halogen;
R⁷ is selected from the group consisting of H, R^{7′}, and —CH₂OR^{7′}, wherein R^{7′} is C₁-C₄ alkyl substituted by one or more halogen, wherein each halogen is independently selected;
R⁸ is selected from the group consisting of H, R^{8′}, and —CH₂OR^{8′}, wherein R^{8′} is C₁-C₄ alkyl substituted by one or more halogen, wherein each halogen is independently selected;
R⁹ is C₁-C₄ alkyl; and
R¹⁰ is C₁-C₄ alkyl,
provided that when R⁷ is H, R^{6a} is H, and R^{6b} is H, then R⁵ is R^{5′}, and provided that the compound is not

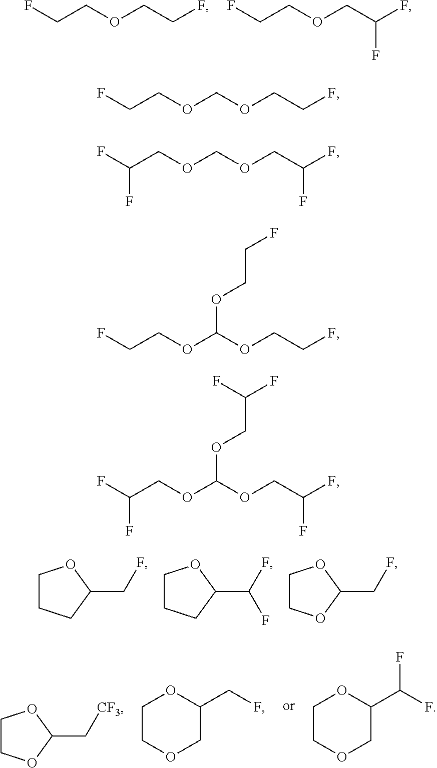

In some embodiments of Formula (I), Formula (II), or Formula (III):
R¹ is C₁-C₄ alkyl substituted by one or more F;
R² is selected from the group consisting of R^{2′} and —OR^{2′}, wherein R^{2′} is C₁-C₄ alkyl substituted by one or more F;
R³ is selected from the group consisting of H, R^{3′}, and —OR^{3′}, wherein R^{3′} is C₁-C₄ alkyl substituted by one or more F;
R⁴ is selected from the group consisting of H, R^{4′}, and —OR^{4′}, wherein R^{4′} is C₁-C₄ alkyl substituted by one or more F;
R⁵ is selected from the group consisting of H, R^{5′}, and —CH₂OR^{5′}, wherein R^{5′} is C₁-C₄ alkyl substituted by one or more F;
R^{6a} is selected from the group consisting of H, F, R^{6′}, and —CH₂OR^{6′}, wherein R^{6′} is C₁-C₄ alkyl substituted by one or more F;
R^{6b} is selected from the group consisting of H and F;
R⁷ is selected from the group consisting of H, R^{7′}, and —CH₂OR^{7′}, wherein R^{7′} is C₁-C₄ alkyl substituted by one or more F; and
R⁸ is selected from the group consisting of H, R^{8′}, and —CH₂OR^{8′}, wherein R^{8′} is C₁-C₄ alkyl substituted by one or more F.

In some embodiments, the compound of Formula (I), Formula (II), or Formula (III) is a compound of Formula (I).

In some embodiments, the compound of Formula (I), Formula (II), or Formula (III) is a compound of Formula (II).

In some embodiments, the compound of Formula (I), Formula (II), or Formula (III) is a compound of Formula (III).

In some embodiments, X is *—(CH₂)ₙO—**, wherein * denotes the point of attachment to the carbon bearing R^{6a} and R^{6b}, and ** denotes the point of attachment to the carbon bearing R⁷. In some embodiments, X is *—(CH₂)ₙ—**, wherein * denotes the point of attachment to the carbon bearing R^{6a} and R^{6b}, and ** denotes the point of attachment to the carbon bearing R⁷. In some embodiments, X is *—O(CHR⁸)—**, wherein * denotes the point of attachment to the carbon bearing R^{6a} and R^{6b}, and ** denotes the point of attachment to the carbon bearing R⁷.

In some embodiments, n is 0. In some embodiments, n is 1. In some embodiments, n is 2. In some embodiments, n is 3.

In some embodiments, R¹ is C₁-C₄ alkyl substituted by one or more F. In some embodiments, R¹ is C₁-C₄ alkyl substituted by 1-4 F. In some embodiments, R¹ is C₁-C₄ alkyl substituted by 1-4 halogen, wherein each halogen is independently selected. In some embodiments, R¹ is selected from the group consisting of

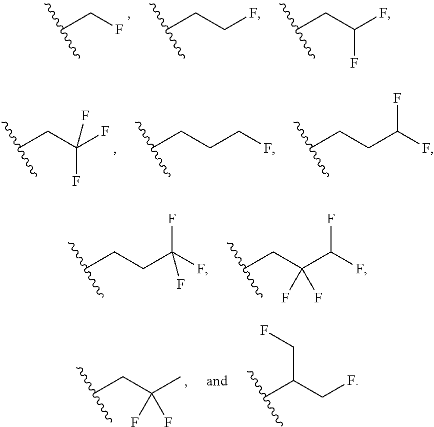

In some embodiments, R¹ is C₁-C₄ alkyl substituted by 1, 2, 3, or 4 F. In some embodiments, the C₁-C₄ alkyl of R¹ is not totally fluorinated. In some embodiments, the C₁-C₄ alkyl of R¹ contains at least one hydrogen atom.

In some embodiments, R² is R^{2′}. In some embodiments, R² is —OR^{2′}. In some embodiments, R^{2′} is C₁-C₄ alkyl substituted by one or more F. In some embodiments, R^{2′} is C₁-C₄ alkyl substituted by 1-4 F. In some embodiments, R^{2′} is C₁-C₄ alkyl substituted by 1-4 halogen, wherein each halogen is independently selected. In some embodiments, R^{2′} is selected from the group consisting of

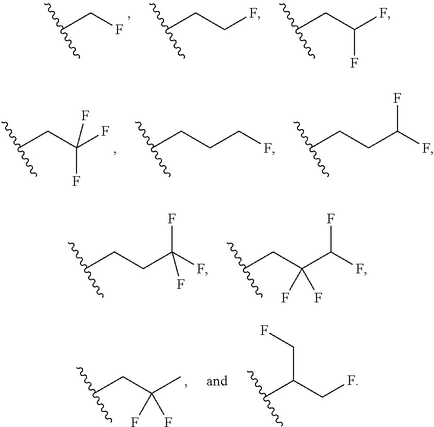

In some embodiments, R^{2′} is C₁-C₄ alkyl substituted by 1, 2, 3, or 4 F. In some embodiments, the C₁-C₄ alkyl of R^{2′} is not totally fluorinated. In some embodiments, the C₁-C₄ alkyl of R^{2′} contains at least one hydrogen atom.

In some embodiments, R³ is H. In some embodiments, R³ is R^{3′}. In some embodiments, R³ is —OR^{3′}. In some embodiments, R^{3′} is C₁-C₄ alkyl substituted by one or more F. In some embodiments, R^{3′} is C₁-C₄ alkyl substituted by 1-4 F. In some embodiments, R^{3′} is C₁-C₄ alkyl substituted by 1-4 halogen, wherein each halogen is independently selected. In some embodiments, R^{3′} is selected from the group consisting of

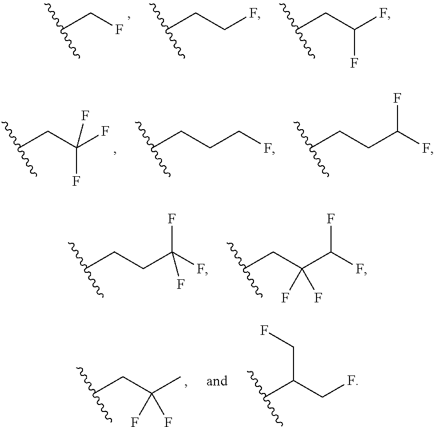

In some embodiments, R^{3′} is C₁-C₄ alkyl substituted by 1, 2, 3, or 4 F. In some embodiments, the C₁-C₄ alkyl of R^{3′} is not totally fluorinated. In some embodiments, the C₁-C₄ alkyl of R^{3′} contains at least one hydrogen atom.

In some embodiments, R⁴ is H. In some embodiments, R⁴ is R^{4′}. In some embodiments, R⁴ is —OR^{4′}. In some embodiments, R^{4′} is C₁-C₄ alkyl substituted by one or more F. In some embodiments, R^{4′} is C₁-C₄ alkyl substituted by 1-4 F. In some embodiments, R^{4′} is C₁-C₄ alkyl substituted by 1-4 halogen, wherein each halogen is independently selected. In some embodiments, R^{4′} is selected from the group consisting of

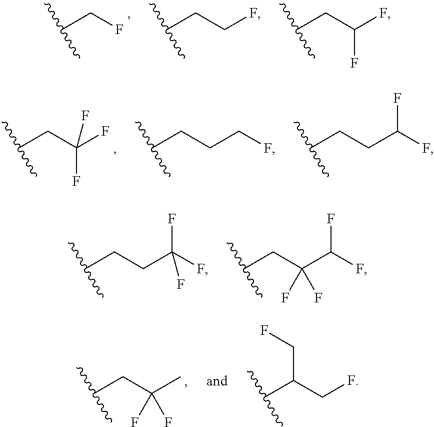

In some embodiments, R⁴ is C₁-C₄ alkyl substituted by 1, 2, 3, or 4 F. In some embodiments, the C₁-C₄ alkyl of R^{4′} is not totally fluorinated. In some embodiments, the C₁-C₄ alkyl of R^{4′} contains at least one hydrogen atom.

In some embodiments, R⁵ is H. In some embodiments, R⁵ is R^{5′}. In some embodiments, R⁵ is —CH₂OR^{5′}. In some embodiments, R^{5′} is C₁-C₄ alkyl substituted by one or more F. In some embodiments, R^{5′} is C₁-C₄ alkyl substituted by 1-4 F. In some embodiments, R^{5′} is C₁-C₄ alkyl substituted by 1-4 halogen, wherein each halogen is independently selected.

In some embodiments, R^{5′} is C₁-C₄ alkyl substituted by 1, 2, 3, or 4 F. In some embodiments, the C₁-C₄ alkyl of R^{5′} is not totally fluorinated. In some embodiments, the C₁-C₄ alkyl of R^{5′} contains at least one hydrogen atom.

In some embodiments R^{6a} is H. In some embodiments R^{6a} is halogen. In some embodiments R^{6a} is F. In some embodiments R^{6a} is R^{6′}. In some embodiments R^{6a} is —CH₂OR^{6′}. In some embodiments, R^{6′} is C₁-C₄ alkyl substituted by 1-4 F. In some embodiments, R^{6′} is C₁-C₄ alkyl substituted by 1-4 halogen, wherein each halogen is independently selected.

In some embodiments, R^{6′} is C₁-C₄ alkyl substituted by 1, 2, 3, or 4 F. In some embodiments, the C₁-C₄ alkyl of R^{6′} is not totally fluorinated. In some embodiments, the C₁-C₄ alkyl of R^{6′} contains at least one hydrogen atom.

In some embodiments R^{6b} is H. In some embodiments R^{6b} is halogen. In some embodiments R^{6b} is F.

In some embodiments R⁷ is H. In some embodiments R⁷ is halogen. In some embodiments R⁷ is F. In some embodiments R⁷ is R^{7′}. In some embodiments R⁷ is —CH₂OR^{7′}. In some embodiments, R^{7′} is C₁-C₄ alkyl substituted by 1-2 F. In some embodiments, R^{7′} is C₁-C₄ alkyl substituted by 1-2 halogen, wherein each halogen is independently selected. In some embodiments, R⁷ is selected from the group consisting of H,

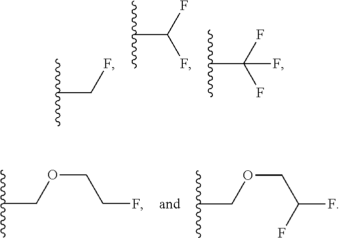

In some embodiments, R^{7′} is C₁-C₄ alkyl substituted by 1, 2, 3, or 4 F. In some embodiments, the C₁-C₄ alkyl of R^{7′} is not totally fluorinated. In some embodiments, the C₁-C₄ alkyl of R^{7′} contains at least one hydrogen atom.

In some embodiments, R⁸ is H. In some embodiments, R⁸ is R^{8′}. In some embodiments, R⁸ is —CH₂OR^{8′}. In some embodiments, R^{8′} is C₁-C₄ alkyl substituted by one or more F. In some embodiments, R^{8′} is C₁-C₄ alkyl substituted by 1-4 F. In some embodiments, R^{8′} is C₁-C₄ alkyl substituted by 1-4 halogen, wherein each halogen is independently selected.

In some embodiments, R^{8′} is C₁-C₄ alkyl substituted by 1, 2, 3, or 4 F. In some embodiments, the C₁-C₄ alkyl of R^{8′} is not totally fluorinated. In some embodiments, the C₁-C₄ alkyl of R^{8′} contains at least one hydrogen atom.

In some embodiments of Formula (I), R² is R^{2′}; R³ is R^{3′}; and R⁴ is R^{4′}. In some embodiments, the compound of Formula (I) is a compound of Formula (I-A):

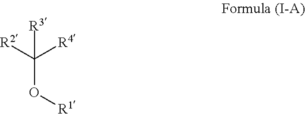

In some embodiments of Formula (I-A), R¹ is C₃-C₄ alkyl substituted by one or more halogen, wherein each halogen is independently selected. In some embodiments, R¹ is C₃-C₄ alkyl substituted by one or more F; R^{2′} is C₁-C₄ alkyl substituted by one or more F; R^{3′} is C₁-C₄ alkyl substituted by one or more F; and R^{4′} is C₁-C₄ alkyl substituted by one or more F.

In some embodiments of Formula (I), R² is —OR^{2′}; R³ is H; and R⁴ is H. In some embodiments, the compound of Formula (I) is a compound of Formula (I-B):

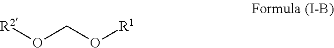

In some embodiments of Formula (I-B), R¹ is C₁-C₄ alkyl substituted by one F; and R^{2′} is C₁-C₄ alkyl substituted by two or more F. In some embodiments of Formula (I-B), R¹ is C₁-C₄ alkyl substituted by two or more F; and R^{2′} is C₁-C₄ alkyl substituted by three or more F. In some embodiments of Formula (I-B), R¹ is C₁-C₄ alkyl substituted by two F; and R^{2′} is C₃-C₄ alkyl substituted by two F.

In some embodiments of Formula (I), R² is —OR^{2′}; R³ is —OR^{3′}; and R⁴ is H. In some embodiments, the compound of Formula (I) is a compound of Formula (I-C):

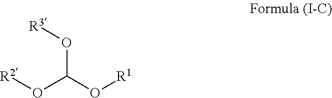

In some embodiments of Formula (I-C), R^{2′} is C₁-C₄ alkyl substituted by one F; and R^{3′} is C₁-C₄ alkyl substituted by two or more F. In some embodiments of Formula (I-C), R^{2′} is C₁-C₄ alkyl substituted by two or more F; and R^{3′} is C₁-C₄ alkyl substituted by three or more F.

In some embodiments of Formula (I), R² is —OR^{2′}; R³ is —OR^{3′}; and R⁴ is —OR^{4′}. In some embodiments, the compound of Formula (I) is a compound of Formula (I-D):

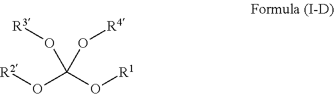

In some embodiments of Formula (I-D), R^{2′} is C₁-C₄ alkyl substituted by one or more F; R^{3′} is C₁-C₄ alkyl substituted by one or more F; and R^{4′} is C₁-C₄ alkyl substituted by one or more F.

In some embodiments of Formula (II), X is *—(CH₂).O—**, wherein * denotes the point of attachment to the carbon bearing R^{6a} and R^{6b}, and ** denotes the point of attachment to the carbon bearing R⁷. In some embodiments, the compound of Formula (II) is a compound of Formula (II-A):

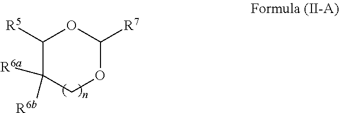

In some embodiments of Formula (II-A), n is 0; R⁵, R^{6a}, and R^{6b} are H; and R⁷ is C₁-C₄ alkyl substituted by two or more halogen, wherein each halogen is independently selected, or R⁷ is —CH₂OR^{7′}, wherein R^{7′} is C₁-C₄ alkyl substituted by one or more halogen, wherein each halogen is independently selected. In some embodiments of Formula (II-A), R⁷ is C₁-C₄ alkyl substituted by two or more F, or R⁷ is —CH₂OR^{7′}, wherein R^{7′} is C₁-C₄ alkyl substituted by one or more F. n is 1; R⁵ is H; and R⁷ is selected from the group consisting of H, R^{7′}, and —CH₂OR^{7′}, wherein R^{7′} is C₁-C₄ alkyl substituted by one or more halogen, wherein each halogen is independently selected. In some embodiments, R⁷ is selected from the group consisting of H, R^{7′}, and —CH₂OR^{7′}, wherein R^{7′} is C₁-C₄ alkyl substituted by one or more F.

In some embodiments of Formula (II), X is *—(CH₂)ₙ—**, wherein * denotes the point of attachment to the carbon bearing R^{6a} and R^{6b}, and ** denotes the point of attachment to the carbon bearing R⁷. In some embodiments, the compound of Formula (II) is a compound of Formula (II-B):

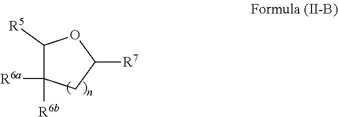

In some embodiments of Formula (II-B), n is 1; R⁵, R^{6a}, and R^{6b} are H; and R⁷ is —CH₂OR^{7′}, wherein R^{7′} is C₁-C₄ alkyl substituted by one or more halogen, wherein each halogen is independently selected. In some embodiments, R⁷ is —CH₂OR^{7′}, wherein R^{7′} is C₁-C₄ alkyl substituted by one or more F. In some embodiments of Formula (II-B), n is 2; R⁵, R^{6a}, and R^{6b} are H; and R⁷ is selected from the group consisting of R^{7′}, and —CH₂OR^{7′}, wherein R^{7′} is C₁-C₄ alkyl substituted by one or more halogen, wherein each halogen is independently selected. In some embodiments, R⁷ is selected from the group consisting of R^{7′}, and —CH₂OR^{7′}, wherein R^{7′} is C₁-C₄ alkyl substituted by one or more F.

In some embodiments of Formula (II), X is *—O(CHR⁸)—**, wherein * denotes the point of attachment to the carbon bearing R^{6a} and R^{6b}, and ** denotes the point of attachment to the carbon bearing R⁷. In some embodiments, the compound of Formula (II) is a compound of Formula (II-C):

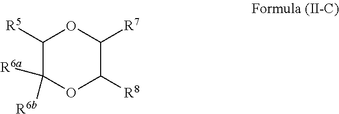

In some embodiments of Formula (II-C), R^{6a} is selected from the group consisting of H, R^{6′}, and —CH₂OR^{6′}, wherein R^{6′} is C₁-C₄ alkyl substituted by one or more halogen, wherein each halogen is independently selected; and R^{6b} is H. In some embodiments, R^{6a} is selected from the group consisting of H, R^{6′}, and —CH₂OR^{6′}, wherein R^{6′} is C₁-C₄ alkyl substituted by one or more F.

In one aspect, provided herein is a compound selected from the group consisting of

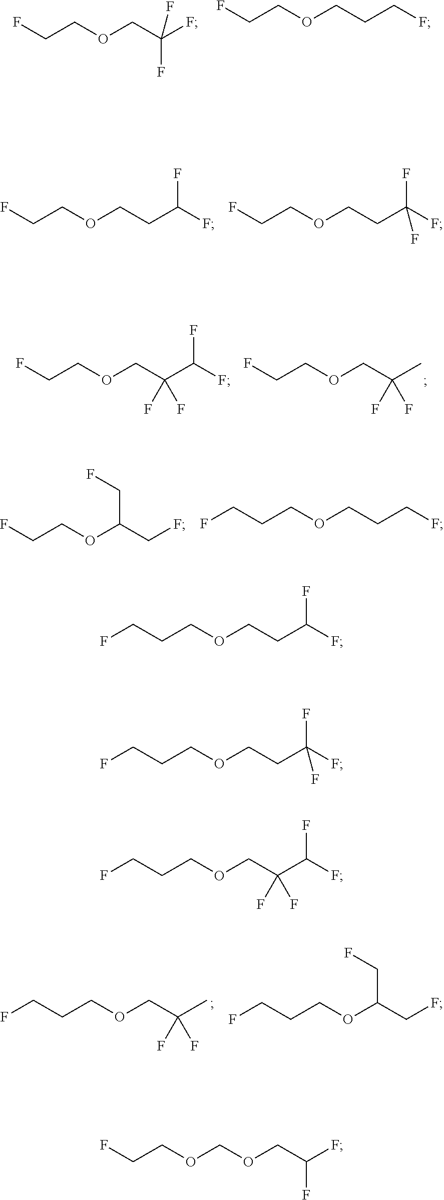

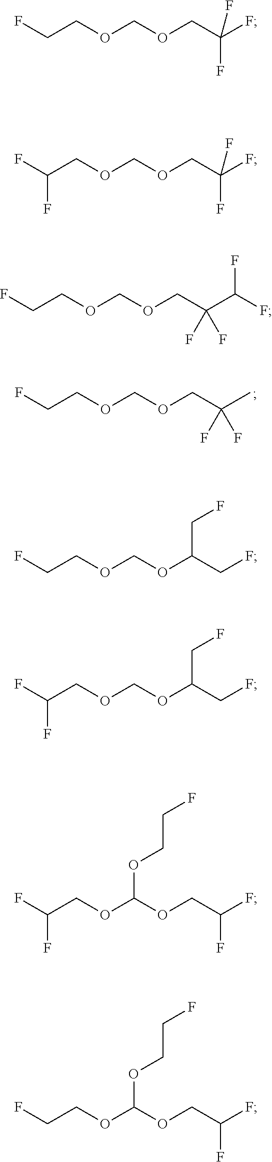

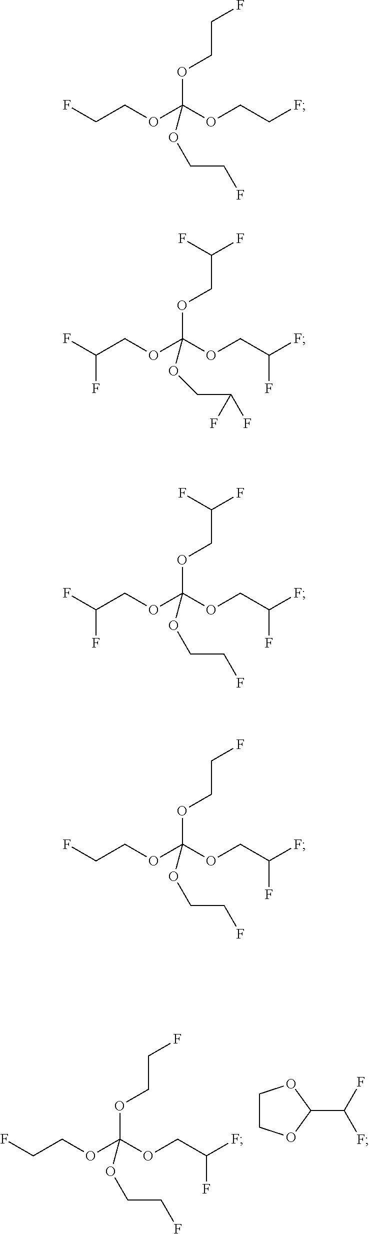

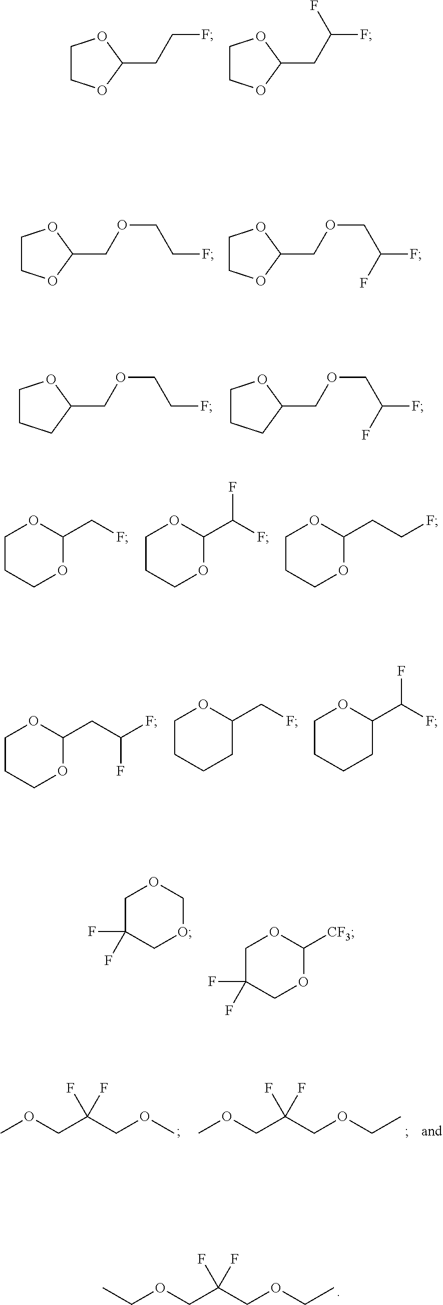

### Methods of Synthesis

In one aspect, provided herein is a method of preparing a compound of Formula (I-A), comprising reacting a compound of Formula (S1)

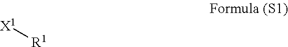

with a compound of Formula (S2)

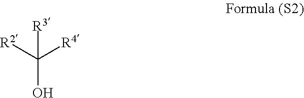

and a base, to provide a compound of Formula (I-A), wherein X¹ is selected from the group consisting of

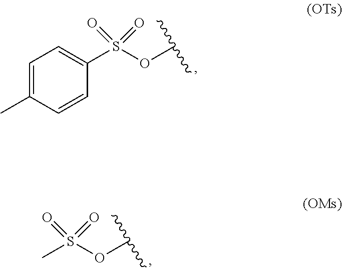

Cl, Br, and I. In some embodiments the base is a methoxide base. In some embodiments, the base is sodium methoxide. In some embodiments the base is a hydride base. In some embodiments, the base is sodium hydride. In some embodiments, the base is a hydroxide base. In some embodiments, the base is sodium hydroxide. In some embodiments, the base is potassium hydroxide. In some embodiments, the reacting is performed in the presence of a solvent. In some embodiments, the solvent is triglyme.

In some embodiments, the method of preparing a compound of Formula (I-A) further comprises preparing a compound of Formula (S1) by reacting a compound of Formula (S3)

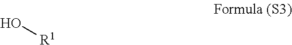

with 4-toluenesulfonyl chloride or methanesulfonyl chloride in the presence of a base to form a compound of Formula (S1). In some embodiments the base is an amine. In some embodiments, the base is triethylamine. In some embodiments the reaction is performed in the presence of a solvent. In some embodiments, the solvent is DCM.

In one aspect, provided herein is a method of preparing a compound of Formula (I-B), comprising reacting a compound of Formula (S3)

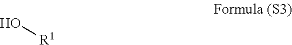

and a compound of formula (S4)

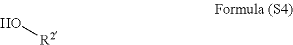

with a compound of Formula (S5)

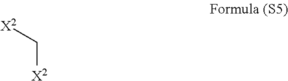

and a base, to provide the compound of Formula (I-B), wherein each X² is independently selected from the group consisting of Cl, Br, and I. In some embodiments, the base is a hydroxide base. In some embodiments, the base is sodium hydroxide. In some embodiments, the base is potassium hydroxide. In some embodiments, the reacting is performed in the presence of a solvent. In some embodiments, the solvent is tetraglyme. In some embodiments, the solvent is tetrahydrofuran (THF).

In one aspect, provided herein is a method of preparing a compound of Formula (I-C), comprising reacting a compound of Formula (S3)

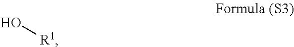

a compound of Formula (S4)

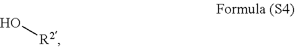

a compound of Formula (S6)

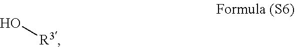

with a compound of Formula (S7)

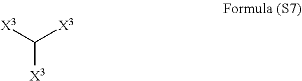

and a base, to provide the compound of Formula (I-C), wherein each X³ is independently selected from the group consisting of Cl, Br, and I. In some embodiments, the base is a hydroxide base. In some embodiments, the base is sodium hydroxide. In some embodiments, the base is potassium hydroxide. In some embodiments, the reacting is performed in the presence of a solvent. In some embodiments, the solvent is tetrahydrofuran (THF).

In one aspect, provided herein is a method of preparing a compound of Formula (I-D), comprising reacting a compound of Formula (S3)

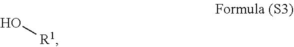

a compound of Formula (S4)

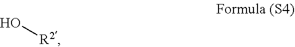

a compound of Formula (S6)

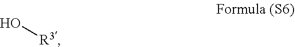

a compound of Formula (S8)

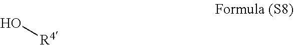

with a compound of Formula (S9)

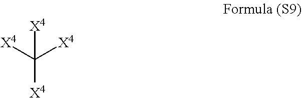

and a base, to provide the compound of Formula (I-D), wherein each X⁴ is independently selected from the group consisting of Cl, Br, and I.

In one aspect, provided herein is a method of preparing a compound of Formula (III), comprising reducing a compound of Formula (S10)

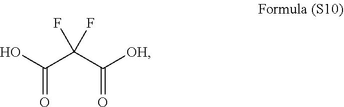

to provide a compound of Formula (S11)

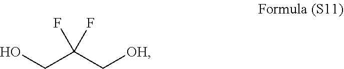

and reacting the compound of Formula (S11) with a compound of Formula (S12)

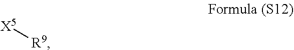

a compound of Formula (S13)

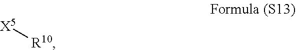

to provide the compound of Formula (III), wherein each X⁵ is independently selected form the group consisting of

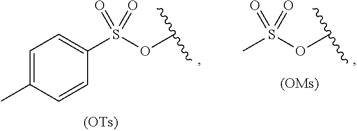

Cl, Br, and I. In some embodiments, the reduction of the compound of Formula (S10) comprises reacting the compound of Formula (S10) with NaBH₄.

### Electrolytes

In one aspect, provided herein is an electrolyte comprising one or more halogenated ether components, wherein each halogenated ether is a compound of Formula (I), Formula (II), or Formula (III). In some embodiments, provided herein is an electrolyte comprising a first halogenated ether component of Formula (I). In some embodiments, provided herein is an electrolyte comprising a first halogenated ether component of Formula (II). In some embodiments, provided herein is an electrolyte comprising a first halogenated ether component of Formula (III).

In some embodiments, provided herein is an electrolyte comprising a single halogenated ether component of Formula (I), Formula (II), or Formula (III), or any embodiments or subformulae thereof. In some embodiments, provided herein is an electrolyte comprising two or more halogenated ether components, wherein each halogenated ether component is independently a compound of Formula (I), Formula (II), or Formula (III), or any embodiments or subformulae thereof. In some embodiments, provided herein is an electrolyte comprising 2, 3, 4, or 5 halogenated ether components, wherein each halogenated ether component is independently a compound of Formula (I), Formula (II), or Formula (III), or any embodiments or subformulae thereof.

In some embodiments, provided herein is an electrolyte comprising a single halogenated ether component of Formula (I) or any embodiments or subformulae thereof. In some embodiments, provided herein is an electrolyte comprising two or more halogenated ether components, wherein each halogenated ether component is independently a compound of Formula (I) or any embodiments or subformulae thereof. In some embodiments, provided herein is an electrolyte comprising 2, 3, 4, or 5 halogenated ether components, wherein each halogenated ether component is independently a compound of Formula (I) or any embodiments or subformulae thereof.

In some embodiments, provided herein is an electrolyte comprising a single halogenated ether component of Formula (II) or any embodiments or subformulae thereof. In some embodiments, provided herein is an electrolyte comprising two or more halogenated ether components, wherein each halogenated ether component is independently a compound of Formula (II) or any embodiments or subformulae thereof. In some embodiments, provided herein is an electrolyte comprising 2, 3, 4, or 5 halogenated ether components, wherein each halogenated ether component is independently a compound of Formula (II) or any embodiments or subformulae thereof.

In some embodiments, provided herein is an electrolyte comprising a single halogenated ether component of Formula (III) or any embodiments or subformulae thereof. In some embodiments, provided herein is an electrolyte comprising two or more halogenated ether components, wherein each halogenated ether component is independently a compound of Formula (III) or any embodiments or subformulae thereof. In some embodiments, provided herein is an electrolyte comprising 2, 3, 4, or 5 halogenated ether components, wherein each halogenated ether component is independently a compound of Formula (III) or any embodiments or subformulae thereof.

In some embodiments, provided herein is an electrolyte comprising a single halogenated ether component selected from the compounds of Table A. In some embodiments, provided herein is an electrolyte comprising two or more halogenated ether components, each of which is independently selected from the compounds of Table A. In some embodiments, provided herein is an electrolyte comprising 2, 3, 4, or 5 halogenated ether components, each of which is independently selected from the compounds of Table A.

TABLE A

| Compound | | |
|---|---|---|
| No. | Compound | Compound Name |
| 1 | 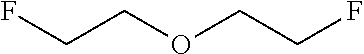 | 1-fluoro-2-(2-fluoroethoxy)- ethane |
| 2 | 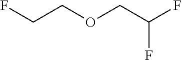 | 1,1-difluoro-2-(2-fluoro- ethoxy)ethane |
| 3 | 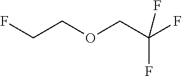 | 1,1,1-trifluoro-2-(2-fluoro- ethoxy)ethane |
| 4 | 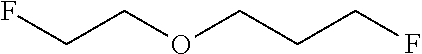 | 1-fluoro-3-(2-fluoroethoxy)- propane |
| 5 | 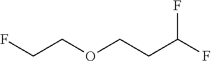 | 1,1-difluoro-3-(2-fluoro- ethoxy)propane |
| 6 | 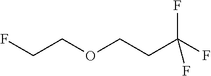 | 1,1,1-trifluoro-3-(2-fluoro- ethoxy)propane |
| 7 | 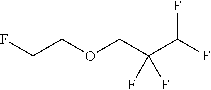 [SMILES: FCCOCC(F)(F)C(F)F] | 1,1,2,2-tetrafluoro-3-(2- fluoroethoxy)propane |
| 8 | 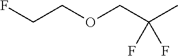 | 2,2-difluoro-1-(2-fluoro- ethoxy)propane |
| 9 | 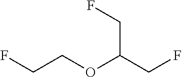 | 1,3-difluoro-2-(2-fluoro- ethoxy)propane |
| 10 | 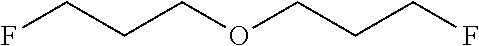 | 1-fluoro-3-(3-fluoropro- poxy)propane |
| 11 | 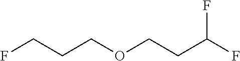 | 1,1-difluoro-3-(3-fluoropro- poxy)propane |
| 12 | 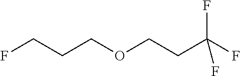 [SMILES: FCCCOCCC(F)(F)F] | 1,1,1-trifluoro-3-(3-fluoro- propoxy)propane |
| 13 | 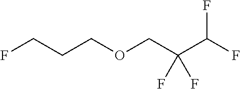 [SMILES: FCCCOCC(F)(F)C(F)F] | 1,1,2,2-tetrafluoro-3-(3- fluoropropoxy)propane |
| 14 | 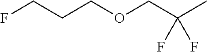 | 2,2-difluoro-1-(3-fluoropro- poxy)propane |
| 15 | 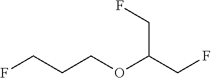 | 1,3-difluoro-2-(3-fluoro- propoxy)propane |
| 16 | 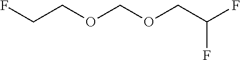 | 1,1-difluoro-2-((2-fluoro- ethoxy)methoxy)ethane |
| 17 | 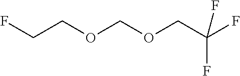 [SMILES: FCCOCOCC(F)(F)F] | 1,1,1-trifluoro-2-((2-fluoro- ethoxy)methoxy)ethane |
| 18 | 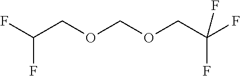 [SMILES: FC(F)COCOCC(F)(F)F] | 2-((2,2-difluoroethoxy)- methoxy)-1,1,1-trifluoro- ethane |
| 19 | 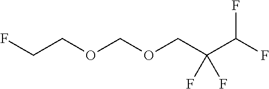 [SMILES: FCCOCOCC(F)(F)C(F)F] | 1,1,2,2-tetrafluoro-3-((2- fluoroethoxy)methoxy)- propane |
| 20 | 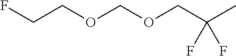 | 2,2-difluoro-1-((2-fluoro- ethoxy)methoxy)propane |
| 21 | 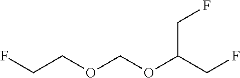 [SMILES: FCCOCOC(CF)CF] | 1,3-difluoro-2-((2-fluoro- ethoxy)methoxy)propane |
| 22 | 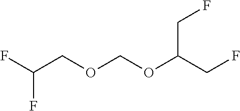 [SMILES: FCC(CF)OCOCC(F)F] | 2-((2,2-difluoroethoxy)- methoxy)-1,3-difluoro- propane |
| 23 | 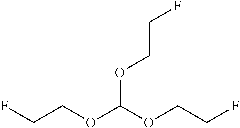 [SMILES: FCCOC(OCCF)OCCF] | tris(2-fluoroethoxy)methane |
| 24 | 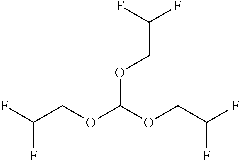 [SMILES: FC(F)COC(OCC(F)F)OCC(F)F] | tris(2,2-difluoroethoxy)- methane |
| 25 | 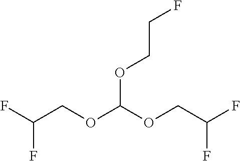 [SMILES: FCCOC(OCC(F)F)OCC(F)F] | 2-((2,2-difluoroethoxy)(2- fluoroethoxy)methoxy)-1,1- difluoroethane |
| 26 | 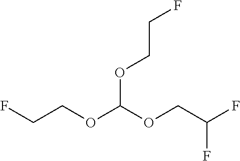 [SMILES: FCCOC(OCCF)OCC(F)F] | 2-(bis(2-fluoroethoxy)meth- oxy)-1,1-difluoroethane |
| 27 | 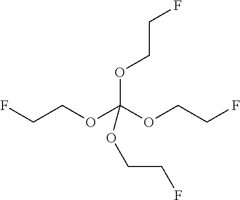 [SMILES: FCCOC(OCCF)(OCCF)OCCF] | tetrakis(2-fluoroethoxy)- methane |
| 28 | 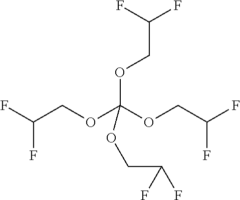 [SMILES: FC(F)COC(OCC(F)F)(OCC(F)F)OCC(F)F] | tetrakis(2,2-difluoroethoxy)- methane |
| 29 | 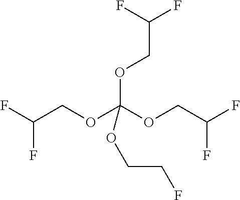 [SMILES: FCCOC(OCC(F)F)(OCC(F)F)OCC(F)F] | 2-(bis(2,2-difluoroethoxy) (2-fluoroethoxy)methoxy)- 1,1-difluoroethane |
| 30 | 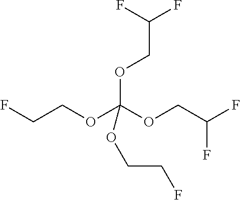 [SMILES: FCCOC(OCCF)(OCC(F)F)OCC(F)F] | bis(2,2-difluoroethoxy)bis- (2-fluoroethoxy)methane |
| 31 | 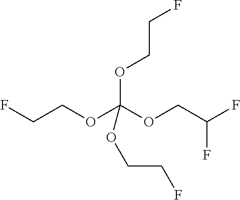 [SMILES: FCCOC(OCCF)(OCCF)OCC(F)F] | 1,1-difluoro-2-(tris(2-fluoro- ethoxy)methoxy)ethane |
| 32 | 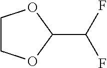 | 2-(difluoromethyl)-1,3- dioxolane |
| 33 | 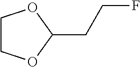 | 2-(2-fluoroethyl)-1,3- dioxolane |
| 34 | 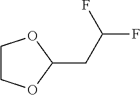 | 2-(2,2-difluoroethyl)-1,3- dioxolane |
| 35 | 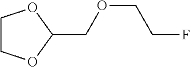 | 2-((2-fluoroethoxy)methyl)- 1,3-dioxolane |
| 36 | 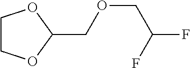 [SMILES: FC(F)COCC1OCCO1] | 2-((2,2-difluoroethoxy)meth- yl)-1,3-dioxolane |
| 37 | 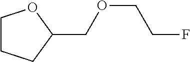 | 2-((2-fluoroethoxy)methyl)- tetrahydrofuran |
| 38 | 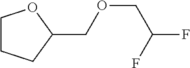 [SMILES: FC(F)COCC1CCCO1] | 2-((2,2-difluoroethoxy)meth- yl)tetrahydrofuran |
| 39 | 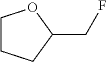 | 2-(fluoromethyl)tetrahydro- furan |
| 40 | 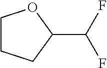 | 2-(difluoromethyl)tetra- hydrofuran |
| 41 | 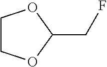 | 2-(fluoromethyl)-1,3- dioxolane |
| 42 | 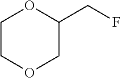 | 2-(fluoromethyl)-1,4-dioxane |
| 43 | 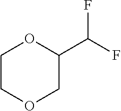 | 2-(difluoromethyl)-1,4- dioxane |
| 44 | 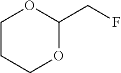 | 2-(fluoromethyl)-1,3-dioxane |
| 45 | 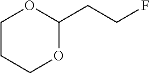 | 2-(2-fluoroethyl)-1,3-dioxane |
| 46 | 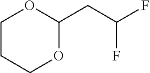 | 2-(2,2-difluoroethyl)-1,3- dioxane |
| 47 | 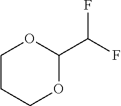 | 2-(difluoromethyl)-1,3- dioxane |
| 48 | 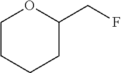 | 2-(fluoromethyl)tetrahydro- 2H-pyran |
| 49 | 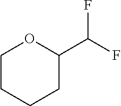 | 2-(difluoromethyl)tetra- hydro-2H-pyran |
| 50 | 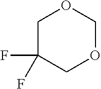 | 5,5-difluoro-1,3-dioxane |
| 51 | 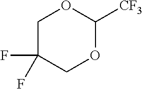 [SMILES: FC1(F)COC(C(F)(F)F)OC1] | 5,5-difluoro-2-(trifluoro- methyl)-1,3-dioxane |
| 52 | 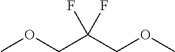 | 2,2-difluoro-1,3-dimethoxy- propane |
| 53 | 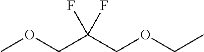 | 1-ethoxy-2,2-difluoro-3- methoxypropane |
| 54 | 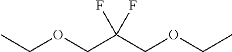 | 1,3-diethoxy-2,2-difluoro- propane |
| 55 | 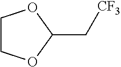 | 2-(2,2,2-trifluoroethyl)-1,3- dioxolane |

In some embodiments, provided herein is an electrolyte comprising a single halogenated ether component selected from compounds 3-22, 25-28, and 44-55 of Table A. In some embodiments, provided herein is an electrolyte comprising two or more halogenated ether components, each of which is independently selected from compounds 3-22, 25-28, and 44-55 of Table A. In some embodiments, provided herein is an electrolyte comprising 2, 3, 4, or 5 halogenated ether components, each of which is independently selected from compounds 3-22, 25-28, or 44-55 of Table A.

In some embodiments, provided herein is an electrolyte comprising a single halogenated ether component selected from compounds 3-22, 25-28, and 44-54 of Table A. In some embodiments, provided herein is an electrolyte comprising two or more halogenated ether components, each of which is independently selected from compounds 3-22, 25-28, and 44-54 of Table A. In some embodiments, provided herein is an electrolyte comprising 2, 3, 4, or 5 halogenated ether components, each of which is independently selected from compounds 3-22, 25-28, or 44-54 of Table A.

In some embodiments, provided herein is an electrolyte comprising a single halogenated ether component selected from compounds 3-22 and 25-31 of Table A. In some embodiments, provided herein is an electrolyte comprising two or more halogenated ether components, each of which is independently selected from compounds 3-22 and 25-31 of Table A. In some embodiments, provided herein is an electrolyte comprising 2, 3, 4, or 5 halogenated ether components, each of which is independently selected from compounds 3-22 and 25-31 of Table A.

In some embodiments, provided herein is an electrolyte comprising a single halogenated ether component selected from compounds 32-38 and 44-51 of Table A. In some embodiments, provided herein is an electrolyte comprising two or more halogenated ether components, each of which is independently selected from compounds 32-38 and 44-51 of Table A. In some embodiments, provided herein is an electrolyte comprising 2, 3, 4, or 5 halogenated ether components, each of which is independently selected from compounds 32-38 and 44-51 of Table A.

In some embodiments, provided herein is an electrolyte comprising a single halogenated ether component selected from compounds 52-54 of Table A. In some embodiments, provided herein is an electrolyte comprising two or more halogenated ether components, each of which is independently selected from compounds 52-54 of Table A. In some embodiments, provided herein is an electrolyte comprising 2, 3, 4, or 5 halogenated ether components, each of which is independently selected from compounds 52-54 of Table A.

In some embodiments, the electrolyte further comprises a solvent component that is not a compound of Formula (I), Formula (II), or Formula (III). In some embodiments, the solvent component is selected from the group consisting of ethylene carbonate (EC); propylene carbonate (PC); dimethyl carbonate (DMC); diethyl carbonate (DEC); ethyl methyl carbonate (EMC); vinyl carbonate (VC); vinyl ethylene carbonate (VEC); fluoroethylene carbonate (FEC); difluoroethylene carbonate (DFEC); 3,3,3-trifluoropropylene carbonate (TFPC); monofluoroethyl methyl carbonate (F1EMC); difluoroethyl methyl carbonate (F2EMC); trifluoroethyl methyl carbonate (F3EMC); bis(2,2,2-trifluoroethyl) carbonate (TFEC); succinic anhydride (SA), butyric anhydride (BA); 1,2-dimethyoxylethane (DME); 1,3-dioxolane (DOL); 1,4-dioxane (DOX); tetrahydrofuran (THF); tetravinyl silane (TVSI); acetonitrile (AN); ethyl acetate (EA); methyl acetate (MA); methyl propanoate (MP); succinonitrile (SN); adiponitrile (ADN); 1,3,6-Hexanetricarbonitrile (HTCN); trimethyl borate (TMB); triphenyl borate (TPB); triethyl borate (TEB); tris(pentafluorophenyl)borane (TPFPB); tris(trimethylsilyl)phosphate (TTSB); tris(2,2,2-trifluoroethyl) borate (TTFEB); trimethyl phosphate (TMP); triethyl phosphate (TEP); tris(trimethylsilyl)phosphate (TTSP); tris(trimethylsilyl)phosphite (TTSPi); tris(2,2,2-trifluoroethyl) phosphate (TFEPa); tris(2,2,2-trifluoroethyl) phosphite (TFEPi); (pentafluorophenyl)diphenyl phosphine (PFPDPP); tris(pentafluorophenyl) phosphine (TPFPP); 1,3,2-dioxathiolane-2,2-dioxide (DTD); 1,3-propanesultone (PS); prop-1-ene-1,3-sultone (PES); propanediol cyclic sulfate (PCS); ethylene sulfite (ES); 1,4-butane sultone (BS); dimethyl sulfoxide (DMSO); methylene methanedisulfonate (MMDS); N,N-Dimethylformamide (DMF); gamma-butyrolactone (BL); bis(2,2,2-trifluoroethyl) ether (BTFE); 1,1,2,2-tetrafluoroethyl-2,2,3,3-tetrafluoropropyl ether (TTE); 1H,1H,5H-octafluoropentyl-1,1,2,2-tetrafluoroethylether (OTE); 2,2,2-trifluoroethyl 1,1,2,2-tetrafluoroethyl ether (HFE); tris(2,2,2-trifluoroethyl) orthoformate (TFEO); 1,2-bis(1,1,2,2-tetrafluoroethoxy)ethane (F8DEE); 1,1,1,2,3,3-hexafluoro-3-(2,2,2-trifluoroethoxy)propane; hexafluoroisopropyl methyl ether; 1-fluoro-2-(2-methoxyethoxy)ethane; 1,1-difluoro-2-(2-methoxyethoxy)ethane; 1,1,1-trifluoro-2-(2-methoxyethoxy)ethane; 1-ethoxy-2-(2-fluoroethoxy)ethane; 2-(2-ethoxyethoxy)-1,1-difluoroethane; 1,2-bis(2-fluoroethoxy)ethane; 1,1-difluoro-2-(2-(2-fluoroethoxy)ethoxy)ethane; 1,1,1-trifluoro-2-(2-(2-fluoroethoxy)ethoxy)ethane; 2-(2-ethoxyethoxy)-1,1,1-trifluoroethane; 1,2-bis(2,2-difluoroethoxy)ethane; 2-(2-(2,2-difluoroethoxy)ethoxy)-1,1,1-trifluoroethane; or 1,2-bis(2,2,2-trifluoroethoxy)ethane; 2-fluoro-1,3-dimethoxypropane; 2-fluoro-1,3-diethoxypropane; 2,2-difluoro-1,3-dimethoxypropane; or 2,2-difluoro-1,3-diethoxypropane; 2-fluoro-1,3-bis(2-fluoroethoxy)propane; 2-fluoro-1,3-bis(2,2-difluoroethoxy)propane; 2-fluoro-1,3-bis(2,2,2-trifluoroethoxy)propane; 2,2-difluoro-1,3-bis(2-fluoroethoxy)propane; 2,2-difluoro-1,3-bis(2,2-difluoroethoxy)propane; 2,2-difluoro-1,3-bis(2,2,2-trifluoroethoxy)propane; and mixtures of any of the foregoing. In some embodiments, provided herein is an electrolyte comprising 2, 3, 4, or 5 components, wherein each component is independently selected from the foregoing list, or is a compound of Formula (I), Formula (II), or Formula (III), or any embodiments or subformulae thereof, provided that at least one component is a compound of Formula (I), Formula (II), or Formula (III), or any embodiments or subformulae thereof.

In some embodiments, the electrolyte does not contain an additional solvent component that is not a compound of Formula (I), Formula (II), or Formula (III). In some embodiments, the electrolyte contains an additional solvent component that is not a compound of Formula (I), Formula (II), or Formula (III), and the proportion of the one or more halogenated ether components in the electrolyte is between about 0.5 wt. % and 99.5 wt, %. In some embodiments, the electrolyte contains an additional solvent component that is not a compound of Formula (I), Formula (II), or Formula (III), and the proportion of the one or more halogenated ether components in the electrolyte is about 0.5 wt. %, about 1 wt. %, about 5 wt. %, about 10 wt. %, about 15 wt. %, about 20 wt. %, about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, about 80 wt. %, about 85 wt. %, about 90 wt. %, about 95 wt. %, about 99 wt. %, or about 99.5 wt. % or a range between any two of the preceding values. In some embodiments, the proportion of the one or more halogenated ether components in the electrolyte is at least about 1 wt. %, at least about 2 wt. %, at least about 3 wt. %, at least about 4 wt. %, at least about 5 wt. %, at least about 10 wt. %, at least about 15 wt. %, at least about 20 wt. %, at least about 25 wt. %, at least about 30 wt. %, at least about 35 wt. %, at least about 40 wt. %, at least about 45 wt. %, at least about 50 wt. %, at least about 55 wt. %, at least about 60 wt. %, at least about 65 wt. %, at least about 70 wt. %, at least about 75 wt. %, at least about 80 wt. %, at least about 85 wt. %, at least about 90 wt. %, at least about 95 wt. %, at least about 98 wt. %, at least about 99 wt. %, at least about 99 wt. %, at least about 99.5 wt. %, or at least about 100 wt. % of the electrolyte. In some embodiments, each of the halogenated ether components in the electrolyte is present in a proportion that is independently selected from between about 0.5 wt. % and about 100 wt. %, provided that the total amount of all halogenated ether components in the electrolyte does not exceed 100 wt. %.

In some embodiments, the electrolyte further comprises one or more salts. In some embodiments, the one or more salts are selected from the group consisting of a lithium salt, a potassium salt, a sodium salt, a cesium salt, a magnesium salt, a zinc salt, a calcium salt, a silver salt, an aluminum salt, a lanthanum salt, and mixtures of any of the foregoing. In some embodiments, the electrolyte comprises one or more salts, each of which is independently selected from the group consisting of lithium bis(fluorosulfonyl)imide (LiFSI); lithium bis(trifluoromethanesulfonyl)imide (LiTFSI); lithium bis(pentafluoroethanesulfonyl)imide (LiBETI); lithium hexafluorophosphate (LiPF₆); lithium hexafluoroarsenate (LiAsF₆); lithium tetrafluoroborate (LiBF₄); lithium bis(oxalato)borate (LiBOB); lithium difluoro(oxalato)borate (LiDFOB); lithium difluorophosphate (LiDFP); lithium difluoro(dioxalato)phosphate (LiDFDOP); lithium tetrafluoro(oxalato)phosphate (LiTFOP); lithium nitrate (LiNO₃); lithium perchlorate (LiClO₄); lithium triflate (LiTf); lithium trifluoroacetate (LiTFA); lithium 4,5-dicyano-2-(trifluoromethyl)imidazole (LiTDI); sodium hexafluorophosphate (NaPF₆); sodium bis(fluorosulfonyl)imide (NaFSI); sodium bis(trifluoromethanesulfonyl)imide (NaTFSI); sodium triflate (NaTf); potassium hexafluorophosphate (KPF₆); potassium bis(fluorosulfonyl)imide (KFSI); potassium bis(trifluoromethanesulfonyl)imide (KTFSI); potassium triflate (KTf); cesium bis(fluorosulfonyl)imide (CsFSI); cesium bis(trifluoromethanesulfonyl)imide (CsTFSI); magnesium bis(trifluoromethanesulfonyl)imide (Mg(TFSI)₂); zinc bis(trifluoromethanesulfonyl)imide (Zn(TFSI)₂); calcium bis(trifluoromethanesulfonyl)imide (Ca(TFSI)₂); silver bis(trifluoromethanesulfonyl)imide (AgTFSI); aluminum bis(trifluoromethanesulfonyl)imide (Al(TFSI)₃); lanthanum bis(trifluoromethanesulfonyl)imide (La(TFSI)₃), and mixtures of any of the foregoing.

In some embodiments, the electrolyte comprises the halogenated ether component of any of the foregoing embodiments, and a salt of any of the foregoing embodiments (e.g., a lithium salt). In some embodiments, the electrolyte comprises one or more halogenated ether components, and the solvent component of the foregoing embodiments, and a salt of any of the foregoing embodiments (e.g., lithium salt). In some embodiments, an amount of the halogenated ether component (or the one or more halogenated ether component and the solvent component) in the electrolyte is at least about 60% by weight of a total weight of the electrolyte, such as at least about 65% by weight, at least about 70% by weight, at least about 75% by weight, or at least about 80% by weight. In some embodiments, the electrolyte consists essentially of the halogenated ether component (or the mixture of one or more halogenated ether components and solvent component) and the salt (e.g., lithium salt). In some embodiments, the electrolyte includes (i) a mixture of one or more halogenated ether components of Formula (I), Formula (II), or Formula (III) or any embodiments or subformulae thereof and one or more solvent components, wherein the solvent component is selected from the group consisting of ethers and carbonates, and (ii) the salt (e.g., lithium salt). Examples of the salts include those elaborated above.

In some embodiments, the electrolyte does not undergo oxidation below a potential of about 3 V vs. Li⁺/Li, about 3.5 V vs. Li⁺/Li, about 4 V vs. Li⁺/Li, about 4.5 V vs. Li⁺/Li, about 5 V vs. Li⁺/Li, about 5.5 V vs. Li⁺/Li, about 6 V vs. Li⁺/Li, about 6.5 V vs. Li⁺/Li, about 7 V vs. Li⁺/Li, about 7.5 V vs. Li⁺/Li, about 8 V vs. Li⁺/Li, about 8.5 V vs. Li⁺/Li, or about 9 V vs. Li⁺/Li. In some embodiments, the electrolyte does not undergo oxidation below a potential of about 6 V vs. Li⁺/Li. In some embodiments, each of the one or more halogenated ether components has a first oxidation potential that is greater than about 3 V vs. Li⁺/Li, greater than about 3.5 V vs. Li⁺/Li, greater than about 4 V vs. Li⁺/Li, greater than about 4.5 V vs. Li⁺/Li, greater than about 5 V vs. Li⁺/Li, greater than about 5.5 V vs. Li⁺/Li, greater than about 6 V vs. Li⁺/Li, greater than about 6.5 V vs. Li⁺/Li, greater than about 7 V vs. Li⁺/Li, greater than about 7.5 V vs. Li⁺/Li, greater than about 8 V vs. Li⁺/Li, greater than about 8.5 V vs. Li⁺/Li, or greater than about 9 V vs. Li⁺/Li. In some embodiments, each of the one or more halogenated ether components has a first oxidation potential that is greater than about 6 V vs. Li⁺/Li.

In some embodiments, the electrolyte does not undergo reduction above a potential of about −3 V vs. Li⁺/Li, about −2.5 V vs. Li⁺/Li, about −2 V vs. Li⁺/Li, about −1.5 V vs. Li⁺/Li, about −1 V vs. Li⁺/Li, about −0.5 V vs. Li⁺/Li, about 0 V vs. Li⁺/Li, about 0.5 V vs. Li⁺/Li, about 1 V vs. Li⁺/Li, about 1.5 V vs. Li⁺/Li, about 2 V vs. Li⁺/Li, about 2.5 V vs. Li⁺/Li, or about 3 V vs. Li⁺/Li. In some embodiments, the electrolyte does not undergo reduction above a potential of about 0 V vs. Li⁺/Li. In some embodiments, each of the one or more halogenated ether components has a first reduction potential that is more negative than about −3 V vs. Li⁺/Li, about −2.5 V vs. Li⁺/Li, about −2 V vs. Li⁺/Li, about −1.5 V vs. Li⁺/Li, about −1 V vs. Li⁺/Li, about −0.5 V vs. Li⁺/Li, about 0 V vs. Li⁺/Li, about 0.5 V vs. Li⁺/Li, about 1 V vs. Li⁺/Li, about 1.5 V vs. Li⁺/Li, about 2 V vs. Li⁺/Li, about 2.5 V vs. Li⁺/Li, or about 3 V vs. Li⁺/Li. In some embodiments, each of the one or more halogenated ether components has a first reduction potential that is more negative than about 0 V vs. Li⁺/Li. In some embodiments the electrolyte has an electrochemical stability window of about −1 V vs. Li⁺/Li to about 7 V vs. Li⁺/Li, about −0.5 V vs. Li⁺/Li to about 6.5 V vs. Li⁺/Li, about 0 V vs. Li⁺/Li to about 6 V vs. Li⁺/Li, about 0.5 V vs. Li⁺/Li to about 5.5 V vs. Li⁺/Li, or about 1 V vs. Li⁺/Li to about 5 V vs. Li⁺/Li. In some embodiments the electrolyte has an electrochemical stability window of about 0 V vs. Li⁺/Li to about 6 V vs. Li⁺/Li. In some embodiments, the one or more halogenated ether components have a collective electrochemical stability window of about −1 V vs. Li⁺/Li to about 7 V vs. Li⁺/Li, about −0.5 V vs. Li⁺/Li to about 6.5 V vs. Li⁺/Li, about 0 V vs. Li⁺/Li to about 6 V vs. Li⁺/Li, about 0.5 V vs. Li⁺/Li to about 5.5 V vs. Li⁺/Li, or about 1 V vs. Li⁺/Li to about 5 V vs. Li⁺/Li. In some embodiments, the one or more halogenated ether components have a collective electrochemical stability window of about 0 V vs. Li⁺/Li to about 6 V vs. Li⁺/Li. In some embodiments, each of the one or more halogenated ether components has an electrochemical stability window of about −1 V vs. Li⁺/Li to about 7 V vs. Li⁺/Li, about −0.5 V vs. Li⁺/Li to about 6.5 V vs. Li⁺/Li, about 0 V vs. Li⁺/Li to about 6 V vs. Li⁺/Li, about 0.5 V vs. Li⁺/Li to about 5.5 V vs. Li⁺/Li, or about 1 V vs. Li⁺/Li to about 5 V vs. Li⁺/Li. In some embodiments, each of the one or more halogenated ether components has an electrochemical stability window of about 0 V vs. Li⁺/Li to about 6 V vs. Li⁺/Li.

### Electrochemical Cells

In one aspect, provided herein is an electrochemical cell comprising an electrolyte as described herein. In some embodiments, provided herein is an electrochemical cell comprising an anode, a cathode, and an electrolyte as described herein. In some embodiments, the electrochemical cell is a battery. In some embodiments, the electrochemical cell is a lithium ion battery.

In some embodiments, the anode of the electrochemical cell comprises an element selected from the group consisting of lithium, sodium, and potassium. In some embodiments, the anode comprises lithium metal, sodium metal, lithium-magnesium alloy, or lithium-aluminum alloy. In some embodiments the anode comprises a surface protection layer comprising fluorine.

In some embodiments, the one or more halogenated ether components suppress or mitigate the formation of undesirable morphologies at the anode or cathode. In some embodiments, the one or more halogenated ether components suppress the formation of dendrites at the anode or cathode. In some embodiments, the one or more halogenated ether components suppress the formation of dendrites at the anode. In some embodiments, the anode comprises lithium metal, and the one or more halogenated ether components suppress the formation of lithium dendrites at the anode.

In additional embodiments, the electrochemical cell is a battery, and includes (1) an anode structure including an anode current collector, (2) a cathode structure including a cathode current collector and a cathode material disposed on the cathode current collector, and (3) the electrolyte of any of the foregoing embodiments disposed between the anode structure and the cathode structure. In some embodiments, the anode structure further includes an anode material disposed on the anode current collector. In some embodiments, the anode material comprises lithium metal, graphite, silicon, graphite/silicon (silicon can in some embodiments be Si, SiOₓ, SiC, or Si₃N₄) composite, sodium metal, hard carbon, and/or potassium metal. In some embodiments, the graphite/silicon (silicon can in some embodiments be Si, SiOₓ, SiC, or Si₃N₄) composite anode includes a weight ratio of graphite/silicon of about 5:95 10:90, 20:80, 30:70, 40:60, 50:50, 60:40, 70:30, 20:80, 90:10, or 95:5. In some embodiments, the cathode material comprises a sulfur-based cathode or an air cathode (e.g., a Li—S, Li-SPAN, or a Li-air battery), a lithium nickel manganese cobalt oxide (e.g., NMC111, NMC532, NMC622, NMC811, NMC900505, NMC95025025, etc.), a lithium nickel cobalt aluminum oxide (NCA), a lithium nickel manganese aluminum oxide (NMA), a lithium nickel manganese cobalt aluminum oxide (NMCA), a lithium nickel oxide (LNO), a lithium nickel manganese oxide (LiNi0.5Mn1.5O4), a lithium cobalt oxide (LCO), a lithium manganese oxide (LMO), a lithium and manganese rich cathode (LMR or LLMO), a lithium iron phosphate (LFP), a lithium cobalt phosphate (LCP), a lithium manganese phosphate (LMP), a lithium manganese iron phosphate (LMFP), a transition metal sulfide (e.g., FeS, FeS2, CuS, MoS2, MoS3, TiS2, TiS4, etc.), sodium vanadium phosphate (Na3V2(PO4)3), sodium copper nickel iron manganese oxide (Na[Cu1/9Ni2/9Fe1/3Mn1/3]O2), Prussian white (R—Na1.92Fe[Fe(CN)6]) or any mixture combination of above cathode materials.

### ENUMERATED EMBODIMENTS

The following enumerated embodiments are representative of some aspects of the invention:

Embodiment 1. A compound of Formula (I), Formula (II), or Formula (III):

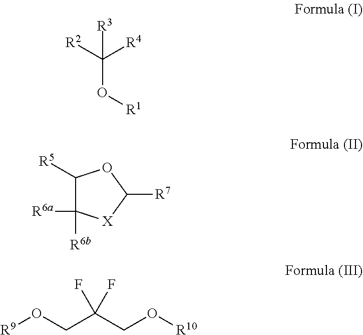

wherein:
X is selected from the group consisting of *—(CH₂)ₙO—**, *—(CH₂)ₙ—**, and *—O(CHR⁸)—**, wherein * denotes the point of attachment to the carbon bearing R^{6a} and R^{6b}, and ** denotes the point of attachment to the carbon bearing R⁷;
n is 0-3;
R¹ is C₁-C₄ alkyl substituted by one or more halogen, wherein each halogen is independently selected;
R² is selected from the group consisting of R^{2′} and —OR^{2′}, wherein R^{2′} is C₁-C₄ alkyl substituted by one or more halogen, wherein each halogen is independently selected;
R³ is selected from the group consisting of H, R^{3′}, and —OR^{3′}, wherein R^{3′} is C₁-C₄ alkyl substituted by one or more halogen, wherein each halogen is independently selected;
R⁴ is selected from the group consisting of H, R^{4′}, and —OR^{4′}, wherein R^{4′} is C₁-C₄ alkyl substituted by one or more halogen, wherein each halogen is independently selected;
R⁵ is selected from the group consisting of H, R^{5′}, and —CH₂OR^{5′}, wherein R^{5′} is C₁-C₄ alkyl substituted by one or more halogen, wherein each halogen is independently selected;
R^{6a} is selected from the group consisting of H, halogen, R^{6′}, and —CH₂OR^{6′}, wherein R^{6′} is C₁-C₄ alkyl substituted by one or more halogen, wherein each halogen is independently selected;
R^{6b} is selected from the group consisting of H and halogen;
R⁷ is selected from the group consisting of H, R^{7′}, and —CH₂OR^{7′}, wherein R^{7′} is C₁-C₄ alkyl substituted by one or more halogen, wherein each halogen is independently selected; and
R⁸ is selected from the group consisting of H, R^{8′}, and —CH₂OR^{8′}, wherein R^{8′} is C₁-C₄ alkyl substituted by one or more halogen, wherein each halogen is independently selected;
R⁹ is C₁-C₄ alkyl;
R¹⁰ is C₁-C₄ alkyl;
provided that when R⁷ is H, R^{6a} is H, and R^{6b} is H, then R⁵ is R^{5′}, and
provided that the compound is not

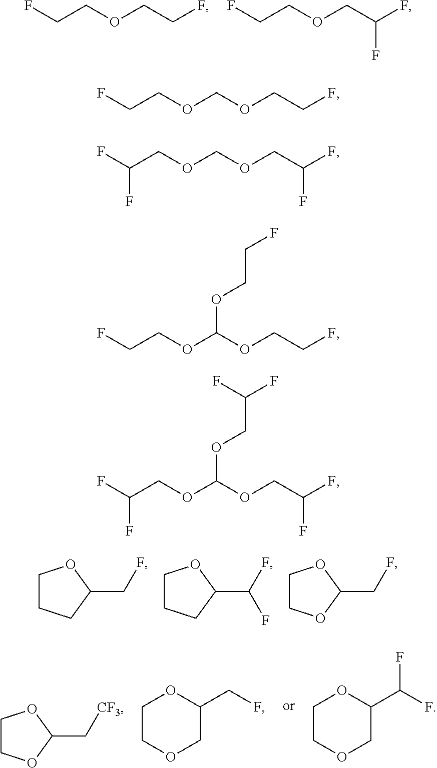

Embodiment 2. The compound of embodiment 1, wherein the compound is a compound of Formula (I).

Embodiment 3. The compound of embodiment 1, wherein the compound is a compound of Formula (II).

Embodiment 4. The compound of embodiment 1, wherein the compound is a compound of Formula (III).

Embodiment 5. The compound of embodiment 1, wherein the compound is a compound of Formula (I), and wherein
R² is R^{2′}.
R³ is R^{3′}; and
R⁴ is R^{4′}.

Embodiment 6. The compound of embodiment 5, wherein R¹ is C₃-C₄ alkyl substituted by one or more halogen, wherein each halogen is independently selected.

Embodiment 7. The compound of embodiment 5 or embodiment 6, wherein
R¹ is C₃-C₄ alkyl substituted by one or more F;
R² is R^{2′}, wherein R^{2′} is C₁-C₄ alkyl substituted by one or more F;
R³ is R^{3′}, wherein R^{3′} is C₁-C₄ alkyl substituted by one or more F; and
R⁴ is R^{4′}, wherein R^{4′} is C₁-C₄ alkyl substituted by one or more F.

Embodiment 8. The compound of embodiment 1, wherein the compound is a compound of Formula (I), and wherein
R² is —OR^{2′};
R³ is H; and
R⁴ is H.

Embodiment 9. The compound of embodiment 8, wherein
R¹ is C₁-C₄ alkyl substituted by one F; and
R² is —OR^{2′}, wherein R^{2′} is C₁-C₄ alkyl substituted by two or more F.

Embodiment 10. The compound of embodiment 8, wherein
R¹ is C₁-C₄ alkyl substituted by two or more F; and
R² is —OR^{2′}, wherein R^{2′} is C₁-C₄ alkyl substituted by three or more F.

Embodiment 11. The compound of embodiment 8, wherein
R¹ is C₁-C₄ alkyl substituted by two F; and
R² is —OR^{2′}, wherein R^{2′} is C₃-C₄ alkyl substituted by two F.

Embodiment 12. The compound of embodiment 1, wherein the compound is a compound of Formula (I), and wherein
R² is —OR^{2′};
R³ is —OR^{3′}; and R⁴ is H.

Embodiment 13. The compound of embodiment 12, wherein
R² is —OR^{2′}, wherein R^{2′} is C₁-C₄ alkyl substituted by one F; and
R³ is —OR^{3′}, wherein R^{3′} is C₁-C₄ alkyl substituted by two or more F.

Embodiment 14. The compound of embodiment 12 wherein
R² is —OR^{2′}, wherein R^{2′} is C₁-C₄ alkyl substituted by two or more F; and
R³ is —OR^{3′}, wherein R^{3′} is C₁-C₄ alkyl substituted by three or more F.

Embodiment 15. The compound of embodiment 1, wherein the compound is a compound of Formula (I), and wherein
R² is —OR^{2′};
R³ is —OR^{3′}; and
R⁴ is —OR^{4′}.

Embodiment 16. The compound of embodiment 15, wherein the compound is a compound of Formula (I), and wherein
R² is —OR^{2′}, wherein R^{2′} is C₁-C₄ alkyl substituted by one or more F;
R³ is —OR^{3′}, wherein R^{3′} is C₁-C₄ alkyl substituted by one or more F; and
R⁴ is —OR^{4′}, wherein R^{4′} is C₁-C₄ alkyl substituted by one or more F.

Embodiment 17. The compound of embodiment 1, wherein the compound is a compound of Formula (II), and wherein X is *—(CH₂).O—**, wherein * denotes the point of attachment to the carbon bearing R^{6a} and R^{6b}, and ** denotes the point of attachment to the carbon bearing R⁷.

Embodiment 18. The compound of embodiment 17, wherein
n is 0;
R⁵, R^{6a}, and R^{6b} are H; and
R⁷ is C₁-C₄ alkyl substituted by two or more halogen, wherein each halogen is independently selected, or
R⁷ is —CH₂OR^{7′}, wherein R^{7′} is C₁-C₄ alkyl substituted by one or more halogen, wherein each halogen is independently selected.

Embodiment 19. The compound of embodiment 18, wherein
R⁷ is C₁-C₄ alkyl substituted by two or more F, or
R⁷ is —CH₂OR^{7′}, wherein R^{7′} is C₁-C₄ alkyl substituted by one or more F.

Embodiment 20. The compound of embodiment 17, wherein
n is 1;
R⁵ is H; and
R⁷ is selected from the group consisting of H, R^{7′}, and —CH₂OR^{7′}, wherein R^{7′} is C₁-C₄ alkyl substituted by one or more halogen, wherein each halogen is independently selected.

Embodiment 21. The compound of embodiment 20, wherein R⁷ is selected from the group consisting of H, R^{7′}, and —CH₂OR^{7′}, wherein R^{7′} is C₁-C₄ alkyl substituted by one or more F.

Embodiment 22. The compound of embodiment 1, wherein the compound is a compound of Formula (II), and wherein X is *—(CH₂)ₙ—**, wherein * denotes the point of attachment to
the carbon bearing R^{6a} and R^{6b}, and ** denotes the point of attachment to the carbon bearing R⁷.

Embodiment 23. The compound of embodiment 22, wherein
n is 1;
R⁵, R^{6a}, and R^{6b} are H; and
R⁷ is —CH₂OR^{7′}, wherein R^{7′} is C₁-C₄ alkyl substituted by one or more halogen, wherein each halogen is independently selected.

Embodiment 24. The compound of embodiment 23, wherein R⁷ is —CH₂OR^{7′}, wherein R^{7′} is C₁-C₄ alkyl substituted by one or more F.

Embodiment 25. The compound of embodiment 21, wherein
n is 2;
R⁵, R^{6a}, and R^{6b} are H; and
R⁷ is selected from the group consisting of R^{7′}, and —CH₂OR^{7′}, wherein R^{7′} is C₁-C₄ alkyl substituted by one or more halogen, wherein each halogen is independently selected.

Embodiment 26. The compound of embodiment 25, wherein R⁷ is selected from the group consisting of R^{7′}, and —CH₂OR^{7′}, wherein R^{7′} is C₁-C₄ alkyl substituted by one or more F.

Embodiment 27. The compound of embodiment 1, wherein the compound is a compound of Formula (II), and wherein X is *—O(CHR⁸)—**, wherein * denotes the point of attachment to the carbon bearing R^{6a} and R^{6b}, and ** denotes the point of attachment to the carbon bearing R⁷.

Embodiment 28. The compound of embodiment 27, wherein
R^{6a} is selected from the group consisting of H, R^{6′}, and —CH₂OR^{6′}, wherein R^{6′} is C₁-C₄ alkyl substituted by one or more halogen, wherein each halogen is independently selected; and
R^{6b} is H.

Embodiment 29. The compound of embodiment 28, wherein R^{6a} is selected from the group consisting of H, R^{6′}, and —CH₂OR^{6′}, wherein R^{6′} is C₁-C₄ alkyl substituted by one or more F.

Embodiment 30. A compound selected from the group consisting of:

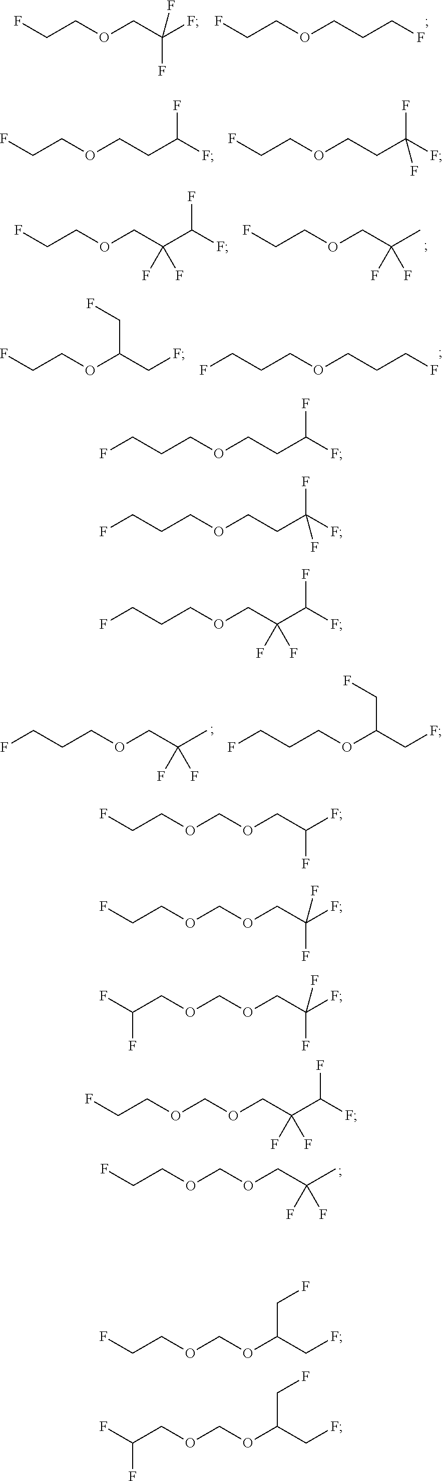

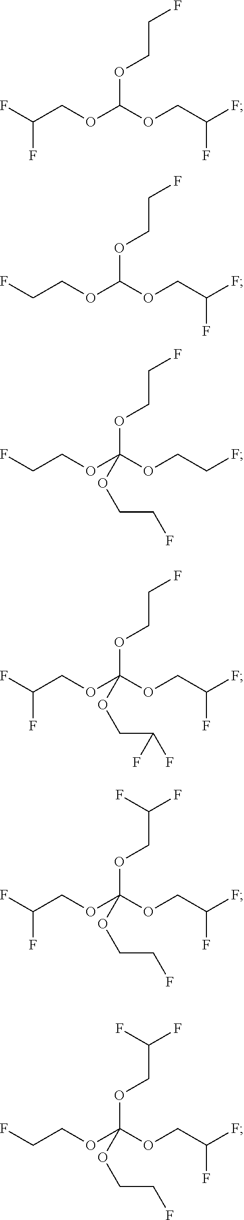

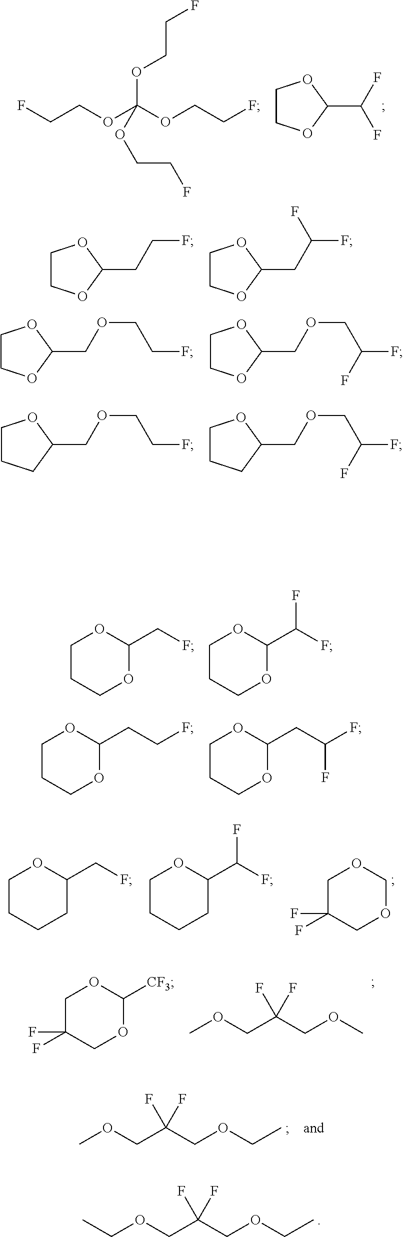

Embodiment 31. An electrolyte comprising a first halogenated ether component of Formula (I), Formula (II), or Formula (III):

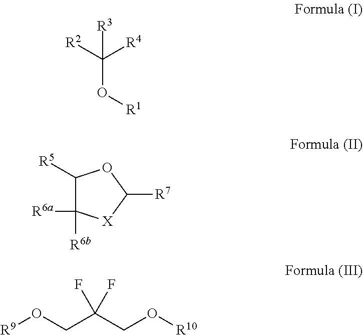

wherein:
X is selected from the group consisting of *—(CH₂)ₙO—**, *—(CH₂)ₙ—**, and *—O(CHR⁸)—**, wherein * denotes the point of attachment to the carbon bearing R^{6a} and R^{6b}, and ** denotes the point of attachment to the carbon bearing R⁷;
n is 0-3;
R¹ is C₁-C₄ alkyl substituted by one or more halogen, wherein each halogen is independently selected;
R² is selected from the group consisting of R^{2′} and —OR^{2′}, wherein R^{2′} is C₁-C₄ alkyl substituted by one or more halogen, wherein each halogen is independently selected;
R³ is selected from the group consisting of H, R^{3′}, and —OR^{3′}, wherein R^{3′} is C₁-C₄ alkyl substituted by one or more halogen, wherein each halogen is independently selected;
R⁴ is selected from the group consisting of H, R^{4′}, and —OR^{4′}, wherein R^{4′} is C₁-C₄ alkyl substituted by one or more halogen, wherein each halogen is independently selected;
R⁵ is selected from the group consisting of H, R^{5′}, and —CH₂OR^{5′}, wherein R^{5′} is C₁-C₄ alkyl substituted by one or more halogen, wherein each halogen is independently selected;
R^{6a} is selected from the group consisting of H, halogen, R^{6′}, and —CH₂OR^{6′}, wherein R^{6′} is C₁-C₄ alkyl substituted by one or more halogen, wherein each halogen is independently selected;
R^{6b} is selected from the group consisting of H and halogen;
R⁷ is selected from the group consisting of H, R^{7′}, and —CH₂OR^{7′}, wherein R^{7′} is C₁-C₄ alkyl substituted by one or more halogen, wherein each halogen is independently selected;
R⁸ is selected from the group consisting of H, R^{8′}, and —CH₂OR^{8′}, wherein R^{8′} is C₁-C₄ alkyl substituted by one or more halogen, wherein each halogen is independently selected; and
R⁹ is C₁-C₄ alkyl;
R¹⁰ is C₁-C₄ alkyl,
provided that when R⁷ is H, R^{6a} is H, and R^{6b} is H, then R⁵ is R^{5′}, and
provided that the compound is not

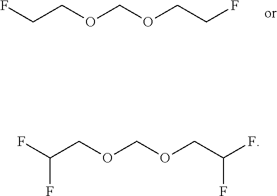 [SMILES: FCCOCOCCF ; FC(F)COCOCC(F)F]

Embodiment 32. The electrolyte of embodiment 31, wherein the first halogenated ether component is a compound of Formula (I).

Embodiment 33. The electrolyte of embodiment 31, wherein the first halogenated ether component is a compound of Formula (II).

Embodiment 34. The electrolyte of embodiment 31, wherein the first halogenated ether component is a compound of Formula (III).

Embodiment 35. The electrolyte of embodiment 31, wherein the first halogenated ether component is a compound of Formula (I), and wherein
R² is R^{2′}.
R³ is R^{3′}; and
R⁴ is R^{4′}.

Embodiment 36. The electrolyte of embodiment 35, wherein
R¹ is C₁-C₄ alkyl substituted by one or more F;
R² is R^{2′}, wherein R^{2′} is C₁-C₄ alkyl substituted by one or more F;
R³ is R^{3′}, wherein R^{3′} is C₁-C₄ alkyl substituted by one or more F; and
R⁴ is R^{4′}, wherein R^{4′} is C₁-C₄ alkyl substituted by one or more F.

Embodiment 37. The electrolyte of embodiment 31, wherein the first halogenated ether component is a compound of Formula (I), and wherein
R² is —OR^{2′};
R³ is H; and
R⁴ is H.

Embodiment 38. The electrolyte of embodiment 37, wherein
R¹ is C₁-C₄ alkyl substituted by one or more F; and
R² is —OR^{2′}, wherein R^{2′} is C₁-C₄ alkyl substituted by one or more F.

Embodiment 39. The electrolyte of embodiment 31, wherein the first halogenated ether component is a compound of Formula (I), and wherein
R² is —OR^{2′};
R³ is —OR^{3′}; and
R⁴ is H.

Embodiment 40. The electrolyte of embodiment 39, wherein
R² is —OR^{2′}, wherein R^{2′} is C₁-C₄ alkyl substituted by one or more F; and
R³ is —OR^{3′}, wherein R^{3′} is C₁-C₄ alkyl substituted by one or more F.

Embodiment 41. The electrolyte of embodiment 31, wherein the first halogenated ether component is a compound of Formula (I), and wherein
R² is —OR^{2′};
R³ is —OR^{3′}; and
R⁴ is —OR^{4′}.

Embodiment 42. The electrolyte of embodiment 41, wherein the first halogenated ether component is a compound of Formula (I), and wherein
R² is —OR^{2′}, wherein R^{2′} is C₁-C₄ alkyl substituted by one or more F;
R³ is —OR^{3′}, wherein R^{3′} is C₁-C₄ alkyl substituted by one or more F; and
R⁴ is —OR^{4′}, wherein R^{4′} is C₁-C₄ alkyl substituted by one or more F.

Embodiment 43. The electrolyte of embodiment 31, wherein the first halogenated ether component is a compound of Formula (II), and wherein X is *—(CH₂).O—**, wherein * denotes the point of attachment to the carbon bearing R^{6a} and R^{6b}, and ** denotes the point of attachment to the carbon bearing R⁷.

Embodiment 44. The electrolyte of embodiment 43, wherein
n is 0;
R⁵, R^{6a}, and R^{6b} are H; and
R⁷ is selected from the group consisting of H, R^{7′}, and —CH₂OR^{7′}, wherein R^{7′} is C₁-C₄ alkyl substituted by one or more F.

Embodiment 45. The electrolyte of embodiment 43, wherein
n is 1;
R⁵ is H; and
R⁷ is selected from the group consisting of H, R^{7′}, and —CH₂OR^{7′}, wherein R^{7′} is
C₁-C₄ alkyl substituted by one or more F.

Embodiment 46. The electrolyte of embodiment 31, wherein the first halogenated ether component is a compound of Formula (II), and wherein X is *—(CH₂)ₙ—**, wherein * denotes the point of attachment to the carbon bearing R^{6a} and R^{6b}, and ** denotes the point of attachment to the carbon bearing R⁷.

Embodiment 47. The electrolyte of embodiment 46, wherein
n is 1;
R⁵, R^{6a}, and R^{6b} are H; and
R⁷ is selected from the group consisting of H, R^{7′}, and —CH₂OR^{7′}, wherein R^{7′} is C₁-C₄ alkyl substituted by one or more F.

Embodiment 48. The electrolyte of embodiment 46, wherein
n is 2;
R⁵, R^{6a}, and R^{6b} are H; and
R⁷ is selected from the group consisting of H, R^{7′}, and —CH₂OR^{7′}, wherein R^{7′} is C₁-C₄ alkyl substituted by one or more F.

Embodiment 49. The electrolyte of embodiment 31, wherein the first halogenated ether component is a compound of Formula (II), and wherein X is *—O(CHR¹)—**, wherein * denotes the point of attachment to the carbon bearing R^{6a} and R^{6b}, and ** denotes the point of attachment to the carbon bearing R⁷.

Embodiment 50. The electrolyte of embodiment 49, wherein
R^{6a} is selected from the group consisting of H, R^{6′}, and —CH₂OR^{6′}, wherein R^{6′} is C₁-C₄ alkyl substituted by one or more F; and
R^{6b} is H.

Embodiment 51. The electrolyte of embodiment 31, wherein the first halogenated ether component is a compound selected from the group consisting of:

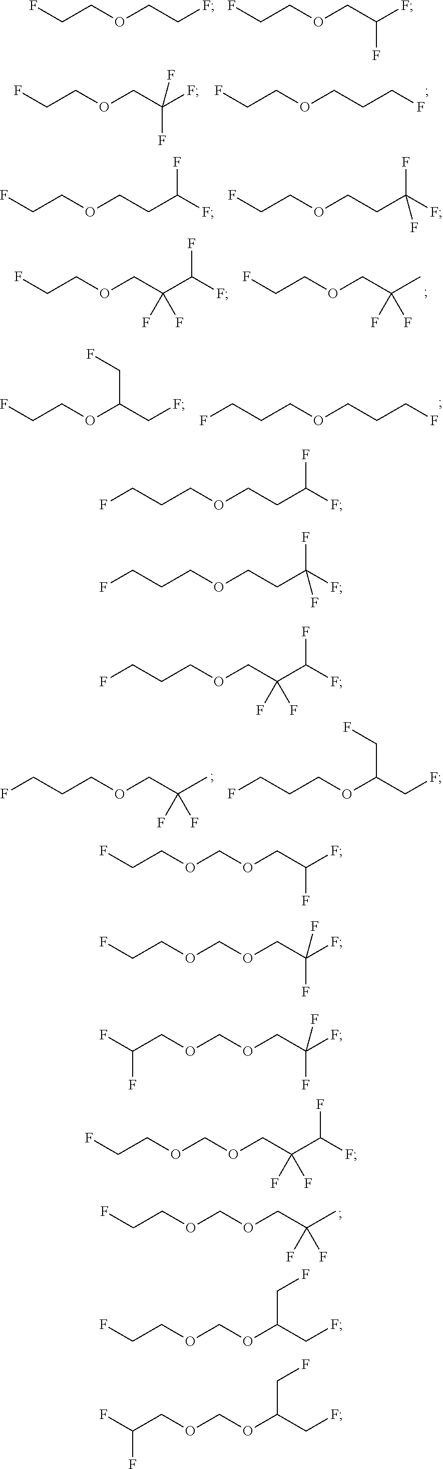

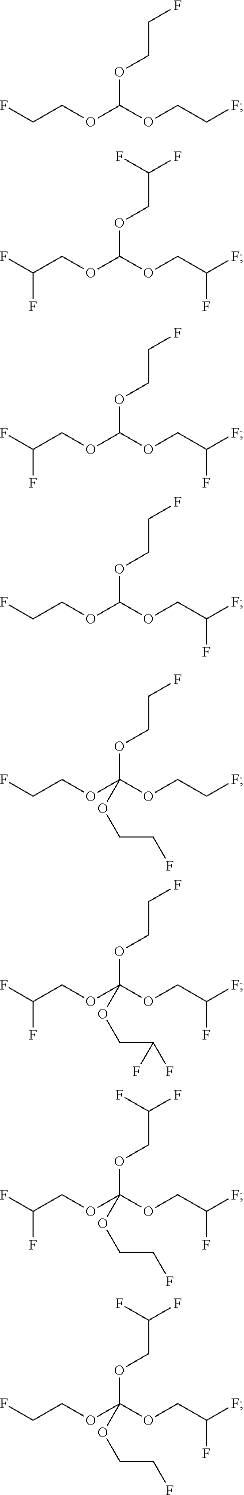

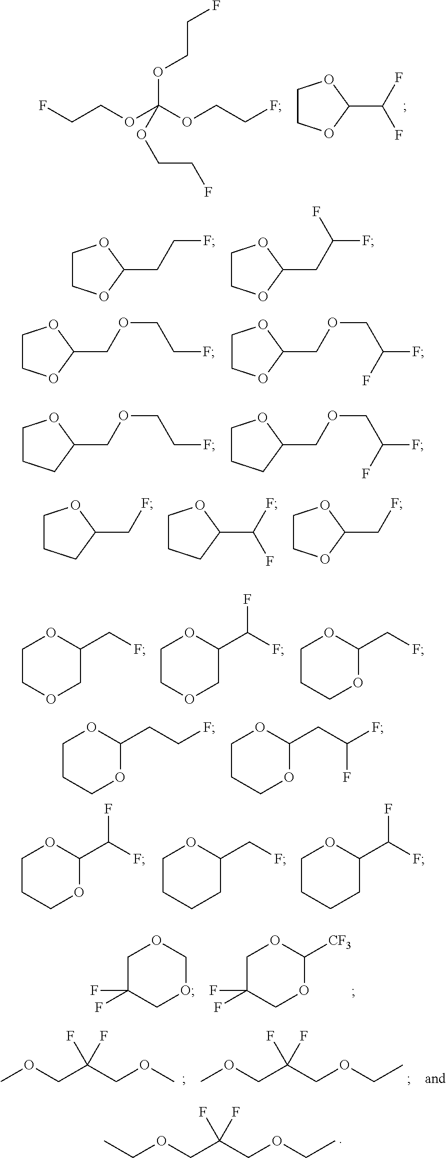

Embodiment 52. The electrolyte of any one of embodiments 31-51, wherein the electrolyte comprises one or more additional halogenated ether components, each of which is independently a compound of Formula (I), Formula (II), or Formula (III).

Embodiment 53. The electrolyte of any one of embodiments 31-52, wherein the electrolyte comprises a solvent component that is not a compound of Formula (I), Formula (II), or Formula (III).

Embodiment 54. The electrolyte of embodiment 53, wherein the solvent component is selected from the group consisting of ethylene carbonate (EC); propylene carbonate (PC); dimethyl carbonate (DMC); diethyl carbonate (DEC); ethyl methyl carbonate (EMC); vinyl carbonate (VC); fluoroethylene carbonate (FEC); difluoroethylene carbonate (DFEC); 3,3,3-trifluoropropylene carbonate (TFPC); monofluoroethyl methyl carbonate (F1EMC); difluoroethyl methyl carbonate (F2EMC); trifluoroethyl methyl carbonate (F3EMC); bis(2,2,2-trifluoroethyl) carbonate (TFEC); 1,2-dimethyoxylethane (DME); 1,3-dioxolane (DOL); 1,4-dioxane (DOX); tetrahydrofuran (THF); 1,3,2-dioxathiolane-2,2-dioxide (DTD); 1,3-propanesultone (PS); acetonitrile (AN); ethyl acetate (EA); methyl acetate (MA); methyl propanoate (MP); succinonitrile (SN); trimethyl phosphate (TMP); triethyl phosphate (TEP); tris(trimethylsilyl)phosphate (TTSP); tris(2,2,2-trifluoroethyl) phosphate (TFEPa); tris(2,2,2-trifluoroethyl) phosphite (TFEPi); prop-1-ene-1,3-sultone (PES); ethylene sulfite (ES); 1,4-butane sultone (BS); dimethyl sulfoxide (DMSO); methylene methanedisulfonate (MMDS); N,N-Dimethylformamide (DMF); gamma-butyrolactone (BL); bis(2,2,2-trifluoroethyl) ether (BTFE); 1,1,2,2-tetrafluoroethyl-2,2,3,3-tetrafluoropropyl ether (TTE); 1H,1H,5H-octafluoropentyl-1,1,2,2-tetrafluoroethylether (OTE); 2,2,2-trifluoroethyl 1,1,2,2-tetrafluoroethyl ether (HFE); tris(2,2,2-trifluoroethyl) orthoformate (TFEO); 1,2-bis(1,1,2,2-tetrafluoroethoxy)ethane (F8DEE); 1-ethoxy-2-(2-fluoroethoxy)ethane (F1DEE); 2-(2-ethoxyethoxy)-1,1-difluoroethane (F2DEE); 1,2-bis(2-fluoroethoxy)ethane (F1F1DEE); 1,1-difluoro-2-(2-(2-fluoroethoxy)ethoxy)ethane (F1F2DEE); 1,1,1-trifluoro-2-(2-(2-fluoroethoxy)ethoxy)ethane (F1F3DEE); 2-(2-ethoxyethoxy)-1,1,1-trifluoroethane (F3DEE); 1,2-bis(2,2-difluoroethoxy)ethane (F4DEE); 2-(2-(2,2-difluoroethoxy)ethoxy)-1,1,1-trifluoroethane (F5DEE); 1,2-bis(2,2,2-trifluoroethoxy)ethane (F6DEE); and mixtures of any of the foregoing.

Embodiment 55. The electrolyte of embodiment 53 or 54, wherein the amount of the solvent component in the electrolyte is between about 0.5 wt. % and about 99 wt. %.

Embodiment 56. The electrolyte of any one of embodiments 31-55, wherein the electrolyte comprises one or more salts.

Embodiment 57. The electrolyte of embodiment 56, wherein the salt is selected from the group consisting of a lithium salt; a potassium salt; a sodium salt; a cesium salt; a magnesium salt; a zinc salt; a calcium salt; a silver salt; an aluminum salt; a lanthanum salt, and mixtures of any of the foregoing.

Embodiment 58. The electrolyte of embodiment 57, wherein the salt is selected from the group consisting of lithium bis(fluorosulfonyl)imide (LiFSI); lithium bis(trifluoromethanesulfonyl)imide (LiTFSI); lithium bis(pentafluoroethanesulfonyl)imide (LiBETI); lithium hexafluorophosphate (LiPF₆); lithium hexafluoroarsenate (LiAsF₆); lithium tetrafluoroborate (LiBF₄); lithium bis(oxalato)borate (LiBOB); lithium difluoro(oxalato)borate (LiDFOB); lithium difluorophosphate (LiDFP); lithium difluoro(dioxalato)phosphate (LiDFDOP); lithium tetrafluoro(oxalato)phosphate (LiTFOP); lithium nitrate (LiNO₃); lithium perchlorate (LiClO₄); lithium triflate (LiTf); lithium trifluoroacetate (LiTFA); lithium 4,5-dicyano-2-(trifluoromethyl)imidazole (LiTDI); sodium hexafluorophosphate (NaPF₆); sodium bis(fluorosulfonyl)imide (NaFSI); sodium bis(trifluoromethanesulfonyl)imide (NaTFSI); sodium triflate (NaTf); potassium hexafluorophosphate (KPF₆); potassium bis(fluorosulfonyl)imide (KFSI); potassium bis(trifluoromethanesulfonyl)imide (KTFSI); potassium triflate (KTf); cesium bis(fluorosulfonyl)imide (CsFSI); cesium bis(trifluoromethanesulfonyl)imide (CsTFSI); magnesium bis(trifluoromethanesulfonyl)imide (Mg(TFSI)₂); zinc bis(trifluoromethanesulfonyl)imide (Zn(TFSI)₂); calcium bis(trifluoromethanesulfonyl)imide (Ca(TFSI)₂); silver bis(trifluoromethanesulfonyl)imide (AgTFSI); aluminum bis(trifluoromethanesulfonyl)imide (Al(TFSI)₃); lanthanum bis(trifluoromethanesulfonyl)imide (La(TFSI)₃), and mixtures of any of the foregoing.

Embodiment 59. An electrochemical cell comprising:
an anode;
a cathode; and
the electrolyte of any one of embodiments 31-58.

Embodiment 60. The electrochemical cell of embodiment 59, wherein the electrochemical cell is a battery.

Embodiment 61. The electrochemical cell of embodiment 59 or 60, wherein the anode comprises an element selected from the group consisting of lithium, sodium, and potassium.

Embodiment 62. The electrochemical cell of any one of embodiments 59-60, wherein the anode comprises lithium metal, sodium metal, lithium-magnesium alloy, or lithium-aluminum alloy.

Embodiment 63. The electrochemical cell of any one of embodiments 59-62, wherein the anode comprises a surface protection layer comprising fluorine.

Embodiment 64. The electrochemical cell of embodiment 59 or 60, wherein the anode comprises a material selected from the group consisting of lithium metal, graphite, silicon, silicon oxide (SiOₓ), graphite/silicon composite, graphite/silicon oxide (SiOₓ) composite, graphite/silicon nitride (Si₃N₄) composite, graphite/silicon carbide (SiC) composite, sodium metal, hard carbon, potassium metal, and mixtures of any of the foregoing.

Embodiment 65. The electrochemical cell of embodiment 59 or 60, wherein the cathode comprises sulfur, a lithium nickel manganese cobalt oxide, a lithium nickel cobalt aluminum oxide (NCA), a lithium nickel manganese aluminum oxide (NMA), a lithium nickel manganese cobalt aluminum oxide (NMCA), a lithium nickel oxide (LNO), a lithium nickel manganese oxide (LiNi_{0.5}Mn_{1.5}O₄), a lithium cobalt oxide (LCO), a lithium manganese oxide (LMO), a lithium and manganese rich cathode (LMR or LLMO), a lithium iron phosphate (LFP), a lithium cobalt phosphate (LCP), a lithium manganese phosphate (LMP), a lithium manganese iron phosphate (LMFP), a transition metal sulfide, a sodium vanadium phosphate (Na₃V₂(PO₄)₃), a sodium copper nickel iron manganese oxide (Na[Cu_{1/9}Ni_{2/9}Fe_{1/3}Mn_{1/3}]O₂), a Prussian white (R—Na_{1.92}Fe[Fe(CN)₆]), and mixtures of any of the foregoing.

Embodiment 66. A method of preparing the compound of embodiment 5, comprising reacting a compound of Formula (S1)

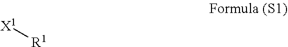

with a compound of Formula (S2)

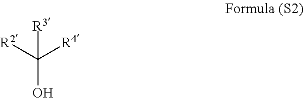

and a base, to provide the compound of embodiment 5, wherein X¹ is selected from the group consisting of

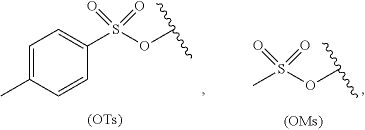

### Cl, Br, and I.

Embodiment 67. A method of preparing the compound of embodiment 8, comprising reacting a compound of Formula (S3)

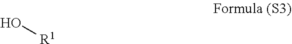

and a compound of formula (S4)

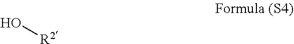

with a compound of Formula (S5)

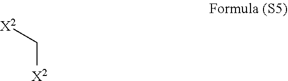

and a base, to provide the compound of embodiment 8, wherein each X² is independently selected from the group consisting of Cl, Br, and I.

Embodiment 68. A method of preparing the compound of embodiment 12, comprising reacting a compound of Formula (S3)

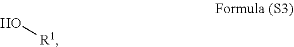

a compound of Formula (S4)

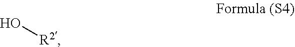

a compound of Formula (S6)

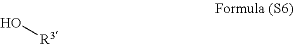

with a compound of Formula (S7)

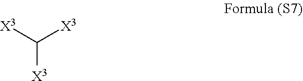

and a base, to provide the compound of embodiment 12, wherein each X³ is independently selected from the group consisting of Cl, Br, and I.

Embodiment 69. A method of preparing the compound of embodiment 15, comprising reacting a compound of Formula (S3)

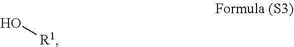

a compound of Formula (S4)

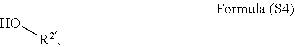

a compound of Formula (S6)

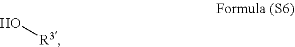

a compound of Formula (S8)

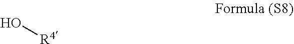

with a compound of Formula (S9)

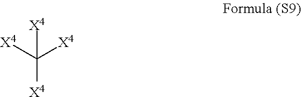

and a base, to provide the compound of embodiment 15, wherein each X⁴ is independently selected from the group consisting of Cl, Br, and I.

Embodiment 70. A method of preparing the compound of embodiment 4, comprising reducing a compound of Formula (S10)

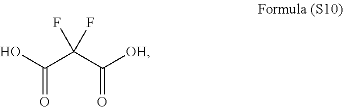

to provide a compound of Formula (S11)

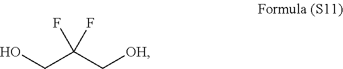

and reacting the compound of Formula (S11) with a compound of Formula (S12)

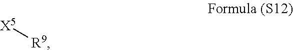

a compound of Formula (S13)

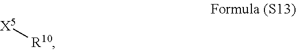

to provide the compound of embodiment 4, wherein each X⁵ is independently selected form the group consisting of

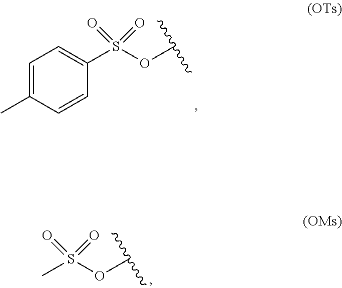

### Cl, Br, and I.

### EXAMPLES

The presently disclosed subject matter will be better understood by reference to the following Examples, which are provided as exemplary of the invention, and not by way of limitation.

### Example 1: Synthesis of Halogenated Ethers of Formula (I)

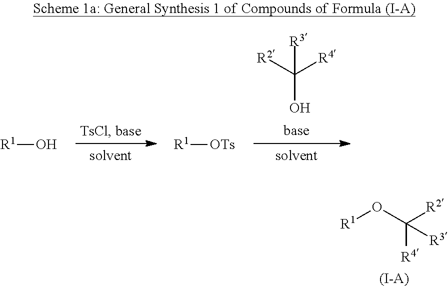

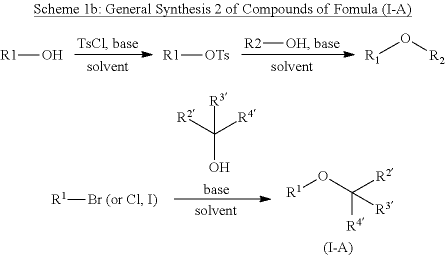

Compounds of Formula (I-A) are prepared according to the general synthesis described in Scheme 1a or Scheme 1b.

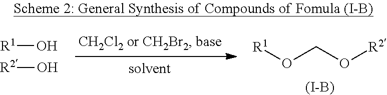

Compounds of Formula (I-B) are prepared according to the general synthesis described in Scheme 2.

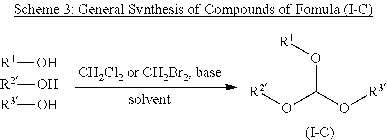

Compounds of Formula (I-C) are prepared according to the general synthesis described in Scheme 3.

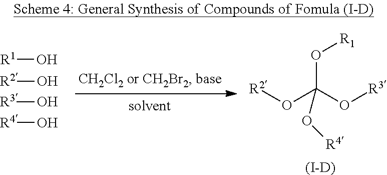

Compounds of Formula (I-D) are prepared according to the general synthesis described in Scheme 4.

### Example 2: Synthesis of Halogenated Ethers of Formula (III)

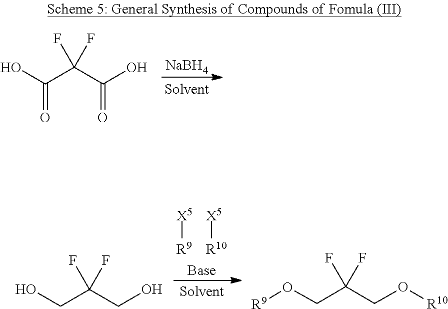

Compounds of Formula (III) are prepared according to the general synthesis described in Scheme 5, wherein each X⁵ is independently selected form the group consisting of

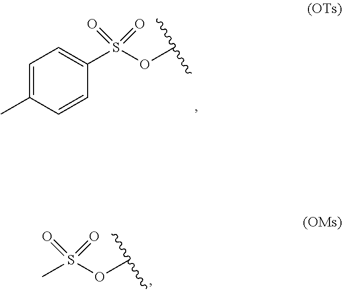

### Cl, Br, and I.

### Example 3: Battery Testing of Electrolytes Containing Halogenated Ethers of Formula (I), Formula (II), or Formula (III)

Electrochemical cells, each containing an electrolyte that includes one or more compounds selected from Table A are prepared. Subsequently, each cell is subjected to electrochemical testing, including electrochemical impedance spectroscopy (EIS), Li⁺ transference number (LTN) testing, and linear scanning voltammetry (LSV) cycling. The Coulombic efficiency (CE) of each cell is also assessed during cycling.

All coin cells are fabricated in an argon-filled glovebox, and one layer of Celgard 2325 is used as a separator. The electrochemical impedance spectroscopy (EIS), Li⁺ transference number (LTN) and linear scanning voltammetry (LSV) cycling are carried out on a Biologic VMP3 system. The cycling tests for coin cells and pouch cells are carried out on an Neware instrument. The EIS measurements are taken over a frequency range of 1 MHz to 100 mHz. For the LTN measurements, 10 mV constant voltage bias is applied to Li∥Li cells. The cathodic cyclic voltammetry tests are carried out over a voltage range of −0.1 to 2 V for one cycle in Li Cu cells, while the anodic LSV tests are over a voltage range of 2.5 to 6.5 V in Li∥ Al cells. For Li∥Li symmetric-cell cycling, 1 mA cm⁻² current density and 1 mAh cm⁻² areal capacity are applied. For Li∥ Cu half-cell Coulombic efficiency (CE) tests, ten pre-cycles between 0 and 1 V are initialized to clean the Cu electrode surface, and then cycling is done by depositing 1 (or 5) mAh cm⁻² of Li onto the Cu electrode followed by stripping to 1 V. The average CE is calculated by dividing the total stripping capacity by the total deposition capacity after the formation cycle. For the Aurbach CE test, a standard protocol is followed: (1) perform one initial formation cycle with Li deposition of 5 mAh cm⁻² on Cu under 0.5 mA cm⁻² current density and stripping to 1 V; (2) deposit 5 mAh cm⁻² Li on Cu under 0.5 mA cm⁻² as a Li reservoir; (3) repeatedly strip/deposit Li of 1 mAh cm⁻² under 0.5 mA cm⁻² for 10 cycles; (4) strip all Li to 1 V. All pouch cells are fabricated in an argon-filled glovebox. The Li∥cathode and Cu∥cathode full cells are cycled with the following method (unless specially listed): after the first two activation cycles at 0.1 C charge/discharge (or 0.1 C charge 0.3 C discharge for anode-free pouch cells), the cells are cycled at different rates. Then a constant-current-constant-voltage protocol is used for cycling: cells are charged to top voltage and then held at that voltage until the current dropped below 0.1 C. The NMC811 coin cells are cycled between 2.8 and 4.4 V and the single-crystal NMC532 pouch cells are cycled between 3.0 and 4.4 V. The Li∥LFP and Cu∥LFP full cells are cycled with the following method (unless specially listed): after the first two activation cycles at 0.1 C charge/discharge (or 0.1 C charge 2 C discharge for anode-free pouch cells), the cells are cycled at different rates. The LFP coin cells are cycled between 2.5 and 3.9 V and the LFP pouch cells are cycled between 2.5 and 3.8 V, or between 2.5 and 3.7 V. All cells are clamped using a specially designed fixture to a rough pressure of 200-300 kPa and cycled under ambient conditions without temperature control.

### Example 4: Tetrahydrofufuryl Fluoride (Compound 39) as Electrolyte Solution in the Use of Anode-Free Lithium Batteries

The chemical structure of Compound 39 is shown below.

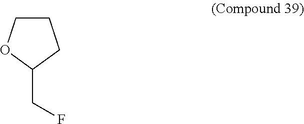

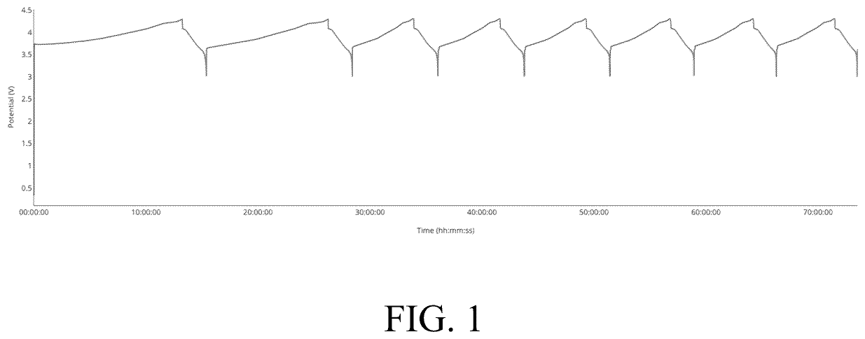

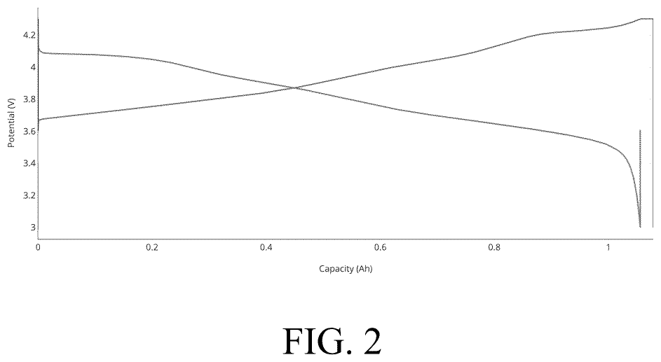

4M lithium bis(fluorosulfonyl)imide (LiFSI) salts were fully dissolved in Compound 39. The as-prepared electrolyte was tested in an anode-free battery with high-Nickle content cathode material. The nominal capacity of this battery was 1 Ah. The total amount of electrolyte added in the dry cell was 1.5 mL. The battery was firstly put through two formation cycles (0.1 C 0.5 D) before the regular cycles (0.2 C 0.5 D) between 3V to 4.3V. The voltage profile for initial cycles is shown in FIG. 1 and the capacity vs. voltage curve of 5^{th} cycle is shown in FIG. 2.

### Example 5: 2-fluoromethyl 1,3-dioxolane (Compound 41) as Electrolyte Solution in the Use of Anode-Free Lithium Batteries

The chemical structure of Compound 41 is shown below.

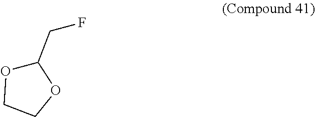

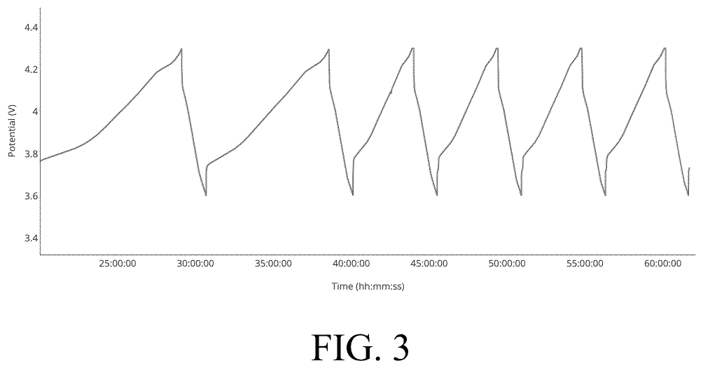

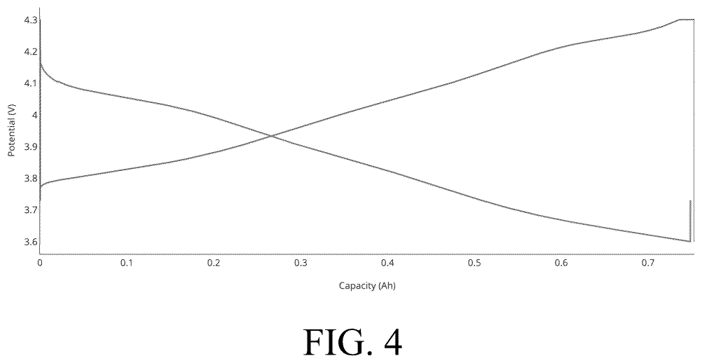

3M LiFSI salts were fully dissolved in Compound 41. The as-prepared electrolyte was examined in an anode-free battery with NMC811 cathode material. The nominal capacity of this battery was 1 Ah. The total amount of electrolyte added in the dry cell was 1.5 mL. The battery was firstly put through two formation cycles (0.1 C 0.5 D) before the regular cycles (0.2 C 0.5 D) between 3.6V to 4.3V. The voltage profile for initial cycles was shown in FIG. 3 and the capacity vs. voltage curve of 5^{th} cycle is shown in FIG. 4.

### Example 6: FDMPr (Compound 52) as Electrolyte Solution in the Use of Anode-Free and Lithium Metal Batteries

The chemical structure of Compound 52 is shown below.

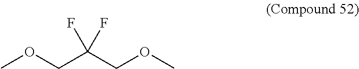

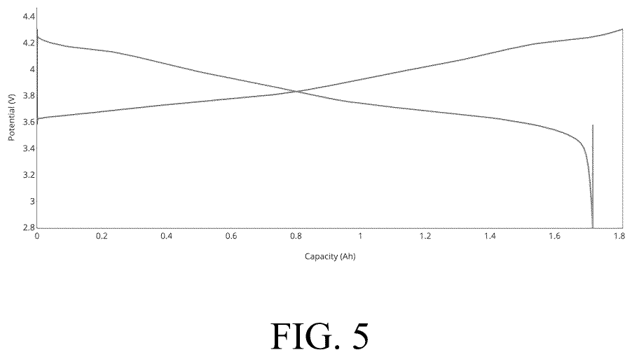

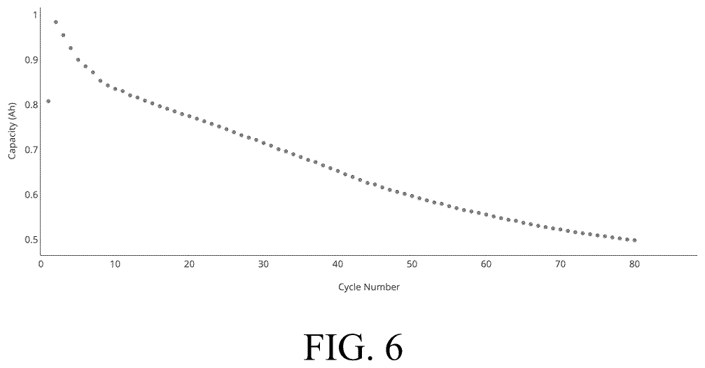

2M LiFSI salts were fully dissolved in Compound 52. The as-prepared electrolyte was firstly tested in an anode-free battery with Ni83 cathode material. The nominal capacity of this battery was 1.1 Ah. The total amount of electrolyte added in the dry cell was 2 mL. The battery was firstly put through two formation cycles (0.1 C 0.5 D) before the regular cycles (0.2 C 0.5 D) between 3V to 4.2V. The capacity vs. voltage curve of 5^{th} cycle is shown in FIG. 5. The same electrolyte was also tested in a lithium metal battery with NMC811 cathode material. The nominal capacity of this lithium metal battery was 2 Ah. The total amount of electrolyte added in the dry cell was 3.5 mL. This battery was also put through two formation cycles (0.1 C 0.1 D) before the regular cycles (0.3 C 1 D) between 2.8V to 4.3V. The capacity vs. cycle number curve of this battery is shown in FIG. 6.

### Example 7: 2-(2,2,2-trifluoroethyl) 1,3-dioxolane (Compound 55) as Electrolyte Solution in the Use of Anode-Free Batteries

The chemical structure of Compound 55 is shown below.

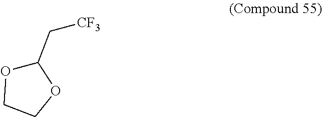

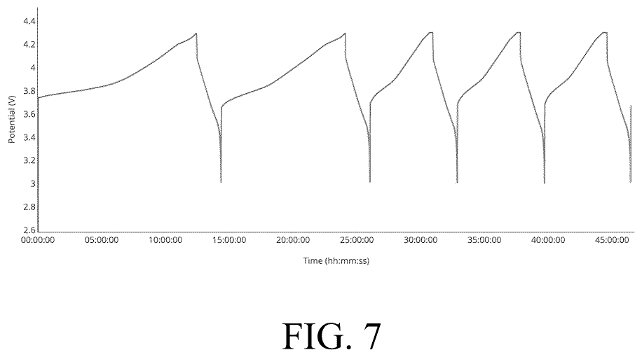

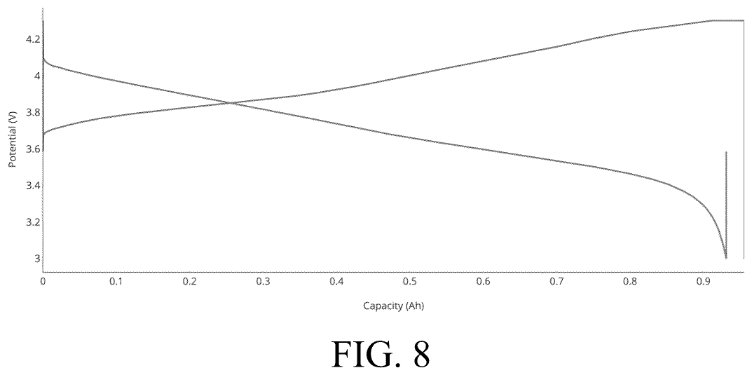

2M LiFSI salts were fully dissolved in Compound 55. The as-prepared electrolyte was tested in an anode-free battery with NMC811 cathode material. The nominal capacity of this battery was 1 Ah. The total amount of electrolyte added in the dry cell was 1.5 mL. The battery was first put through two formation cycles (0.1 C 0.5 D) before the regular cycles (0.2 C 0.5 D) between 3V to 4.3V. The voltage profile for initial cycles is shown in FIG. 7 and the capacity vs. voltage curve of 5^{th} cycle is shown in FIG. 8.

### Example 8: 1,2-bis(2-fluoroethoxy)ethane (Compound 1) as Electrolyte Solution in the Use of Lithium Metal Batteries

The chemical structure of Compound 1 is shown below.

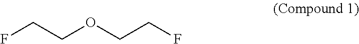

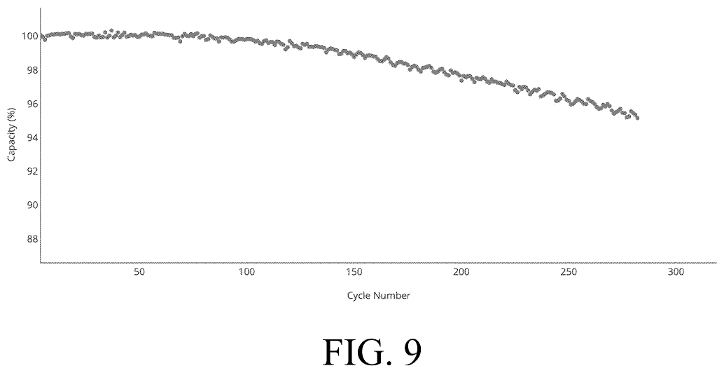

2M LiFSI salts were fully dissolved in Compound 1. The as-prepared electrolyte was tested in a lithium metal battery with NMC811 cathode material. The nominal capacity of this battery was 5 Ah. The total amount of electrolyte added in the dry cell was 8 mL. The battery was allowed to rest for 48 hours at room temperature after electrolyte injection for best wetting results. The battery was then put through two formation cycles (0.1 C 0.1 D) before the regular cycles (0.3 C 1 D) between 2.8V to 4.3V. The capacity vs. cycle number curve is shown in FIG. 9.

### Example 9: 1,1-difluoro-2-(2-fluoroethoxy)ethane (Compound 2) as Electrolyte Solution in the Use of Lithium Metal Batteries

The chemical structure of Compound 2 is shown below.

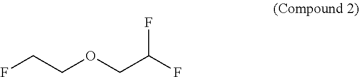

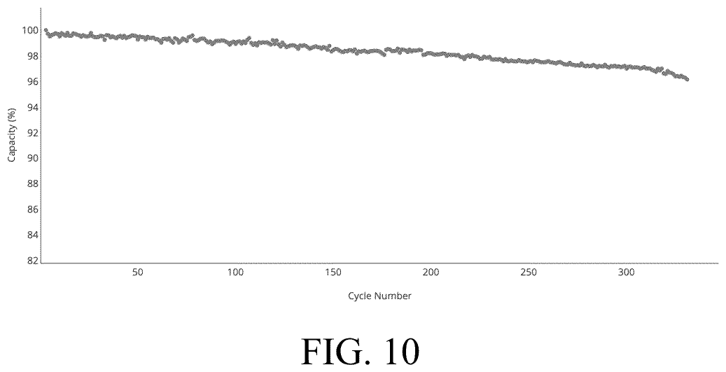

2M LiFSI salts were fully dissolved in pure Compound 2. The as-prepared electrolyte was tested in a lithium metal battery with NMC811 cathode material. The nominal capacity of this battery was 5 Ah. The total amount of electrolyte added in the dry cell was 8 mL. The battery was allowed to rest for 48 hours at room temperature after electrolyte injection for best wetting results. The battery was then put through two formation cycles (0.1 C 0.1 D) before the regular cycles (0.3 C 1 D) between 2.8V to 4.3V. The capacity vs. cycle number curve is shown in FIG. 10.

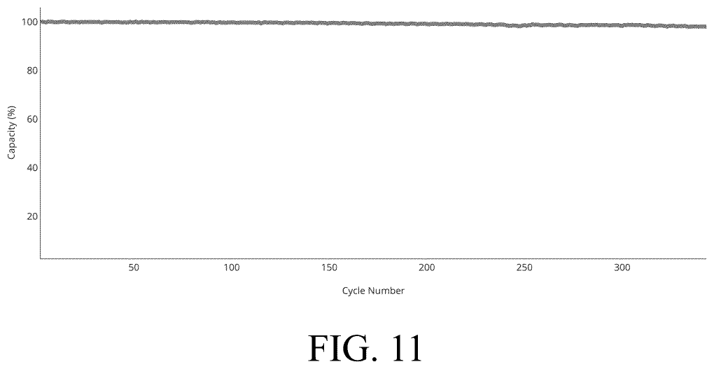

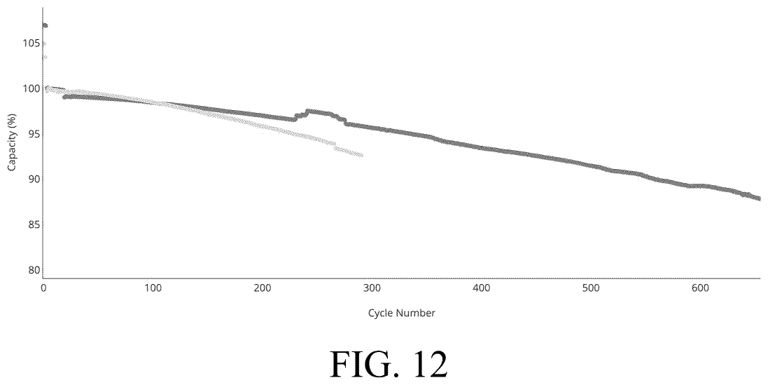

Different recipes of Compound 2 and 2-(2-(2,2-difluoroethoxy)ethoxy)-1,1,1-trifluoroethane (F5) binary solvent were tested. For example, 2M LiFSI salts and 0.1M LiTFSI were dissolved together in a binary Compound 2/F5 (1:5 v/v) solution. The as-prepared electrolyte was tested in a lithium metal battery with NMC811 cathode material. The nominal capacity of this battery was 5 Ah. The total amount of electrolyte added in the dry cell was 8 mL. The battery was allowed to rest for 48 hours at room temperature after electrolyte injection for best wetting results. The battery was then put through two formation cycles (0.1 C 0.1 D) before the regular cycles (0.3 C 1 D) between 2.8V to 4.3V. The capacity vs. cycle number curve is shown in FIG. 11. Two more identical batteries were also cycled under elevated temperature (45° C. and 60° C.), as shown in FIG. 12.

### Example 10: 1,1-difluoro-2-(2-fluoroethoxy)ethane (Compound 2) as a Component of High Entropy Electrolyte in the Use of Anode-Free Batteries

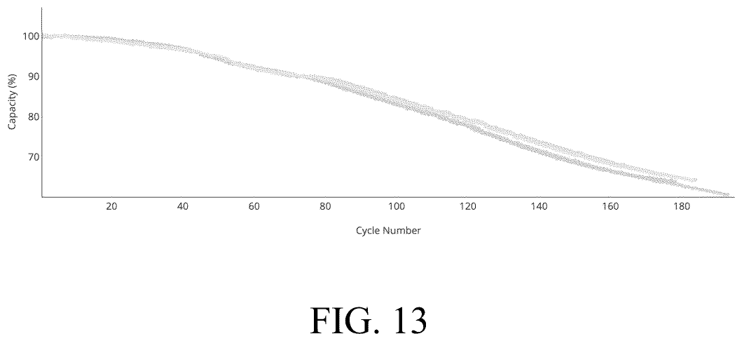

Multiple high entropy electrolytes were prepared with a blend of Compound 2 and F5. 1,1,2,2-tetrafluoroethyl-2,2,3,3-tetrafluoropropyl ether (TTE) serving as diluent in the electrolyte. The concentration of LiFSI salts was controlled at 3M for all groups. Four different electrolyte compositions were tested: (1) Compound 2: F5=1:1 and LiFSI:TTE=1:1; (2) Compound 2: F5=1:2 and LiFSI:TTE=1:1; (3) Compound 2: F5=1:1 and LiFSI:TTE=2:1; (4) Compound 2: F5=1:2 and LiFSI:TTE=2:1. The as-prepared electrolytes were tested in identical anode-free batteries with Ni83 cathode material. The nominal capacity of this battery was 1.1 Ah. The total amount of electrolyte added in the dry cell was 2 mL. The battery was then put through two formation cycles (0.1 C 0.1 D) before the regular cycles (0.3 C 1 D) between 3.6V to 4.3V. FIG. 13 shows four different examples of high entropy electrolytes with their capacity retention curves over 180 cycles.

### Example 11: F3EO (Compound 23) as Electrolyte Solution in the Use of Anode-Free Lithium Batteries

The structure of Compound 23 is shown below.

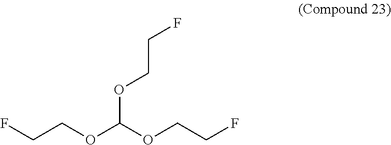 [SMILES: FCCOC(OCCF)OCCF]

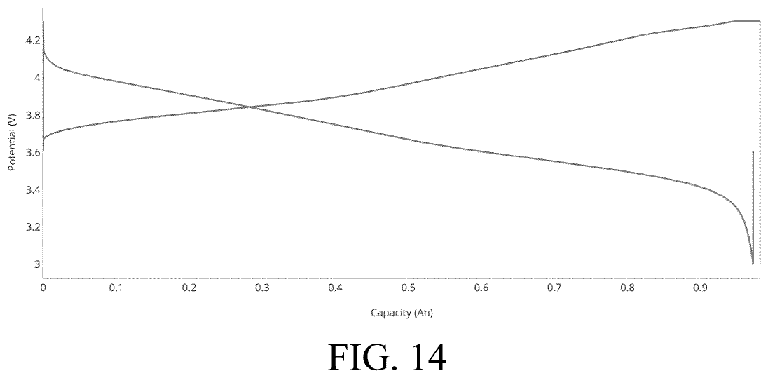

2M LiFSI salts were fully dissolved in Compound 23. The as-prepared electrolyte was tested in an anode-free battery with NMC811 cathode material. The nominal capacity of this battery was 1 Ah. The total amount of electrolyte added in the dry cell was 2 mL. The battery was first put through two formation cycles (0.1 C 0.5 D) before the regular cycles (0.2 C 0.5 D) between 3V to 4.3V. The capacity vs. voltage curve of 5^{th} cycle is shown in FIG. 14.

### Example 12: Commercial Non-Fluorinated Ether, 1,2-Dimethoxyethane, (DME) as Electrolyte Solution in the Use of Anode-Free Lithium Batteries

As a control, an anode-free battery was prepared and tested using the commercial non-fluorinated ether 1,2-dimethoxyethane, (DME). The structure of DME is shown below.

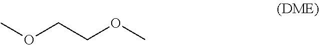

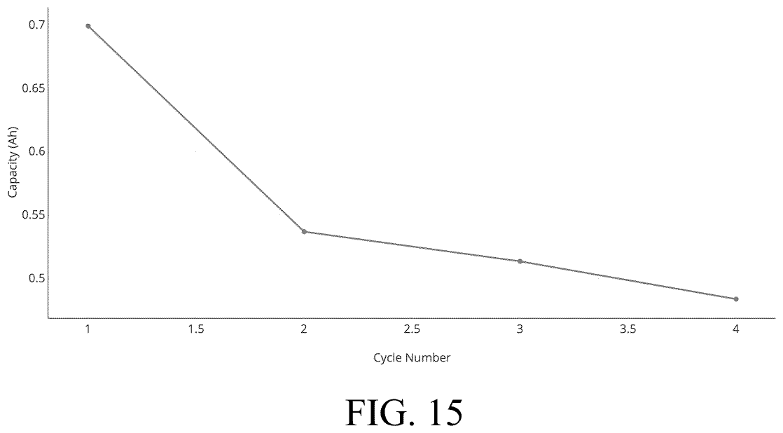

3M LiFSI salts were fully dissolved in DME. The as-prepared electrolyte was tested in an anode-free battery with NMC811 cathode material. The nominal capacity of this battery was 1 Ah. The total amount of electrolyte added in the dry cell was 2 mL. The battery was first put through two formation cycles (0.1 C 0.5 D) before the regular cycles (0.2 C 0.5 D) between 3V to 4.3V. The capacity retention vs. cycle number is shown in FIG. 15. The fluorinated ether electrolytes tested in Examples 3-11 showed improved efficacy cell performance when compared to the non-fluorinated DME-based electrolyte. In contrast to the fluorinated ether electrolytes tested in Examples 3-11, which retain about 70% of their original capacity for at least 30 (e.g., Example 6) and up to over 600 cycles (e.g., Example 9), the non-fluorinated DME based electrolyte could only be cycled for 4 cycles before dropping below 70% of the original capacity and cell failure.

### Example 13: Synthesis of 1-fluoro-2-(2-fluoroethoxy)ethane (Compound 1)

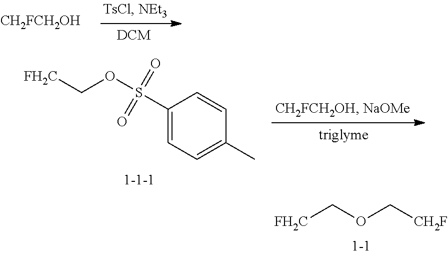

### Step 1: Synthesis of 2-fluoroethyl 4-methylbenzenesulfonate (Intermediate 1-1-1)

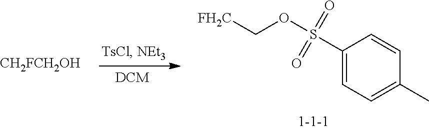

TABLE S13-1

| | | Added | | |
|---|---|---|---|---|
| Reactant | M.W. | amount | | Equiv. |
| 2-fluoroethanol | 64.06 | 300 | g | 1.05 |
| TsCl | 190.65 | 850 | g | 1.0 |
| triethylamine | 101.19 | 680 | g | 1.5 |
| DCM (solvent) | | 2.4 | L | |

In a reactor were added 2-fluoroethanol, triethylamine and DCM, and the mixed solution was stirred at 0° C. for 30 minutes. TsCl was added in batches and the system was stirred at room temperature for 6 hours. After the reaction, NH₄Cl aqueous solution was used to wash the DCM layer to remove excess triethylamine. The organic layer was further washed with saturated NaCl solution and dried with Na₂SO₄. Rotary evaporation was then used to obtain a red oil as the crude product, which was used in the next step without further purification. Yield was ˜97%.

### Step 2: Synthesis of 1-fluoro-2-(2-fluoroethoxy)ethane (Compound 1)

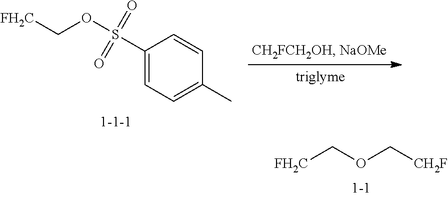

TABLE S13-2

| | | Added | | |
|---|---|---|---|---|
| Reactant | M.W. | amount | | Equiv. |
| 1-1-1 | 218.21 | 360 | g | 1.0 |
| 2-fluoroethanol | 64.06 | 138 | g | 1.3 |
| NaOMe | 54.02 | 178 | g | 2.0 |
| Triglyme (solvent) | | 1.8 | L | |

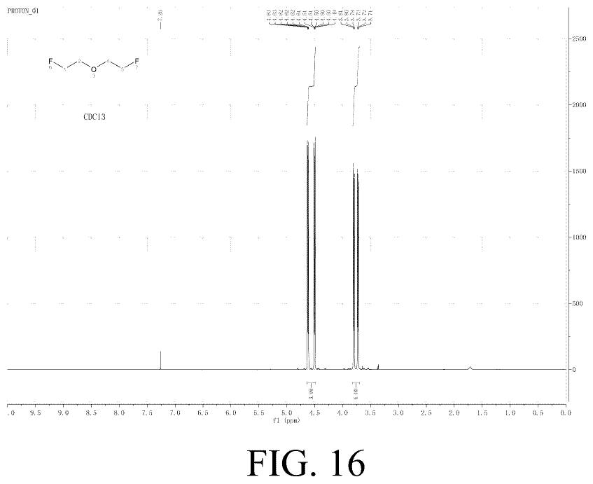

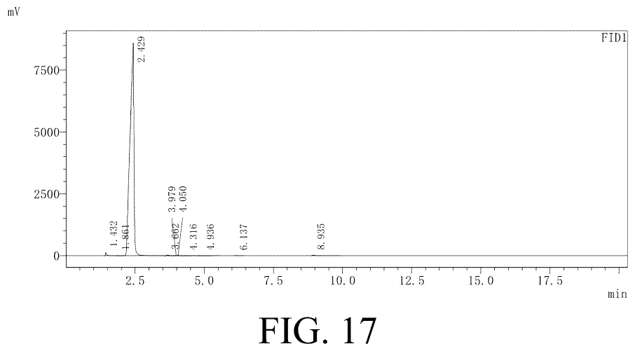

Under nitrogen atmosphere, in a reactor were added NaOMe and triglyme. The suspension was cooled to 0° C. While stirring, 2-fluoroethanol was added slowly into the suspension. After the addition of 2-fluoroethanol, Intermediate 1-1-1 was added slowly into the suspension. After stirring at 0° C. for 30 min, the suspension was warmed up and allowed to react overnight. After the reaction, water was added to quench excess NaOMe. DCM was used to extract the system. The organic layer was washed by saturated NaCl solution and dried with Na₂SO₄. Rotary evaporation was used to obtain concentrated crude product. The crude product underwent vacuum distillation to obtain a colorless liquid as the final product with a purity of 99.7%. Final yield after distillation was ˜80%. ¹H-NMR and GC-MS for the resulting product (Compound 1) are shown in FIG. 16 and FIG. 17, respectively.

### Example 14: Synthesis of 1,1-difluoro-2-(2-fluoroethoxy)ethane (Compound 2)

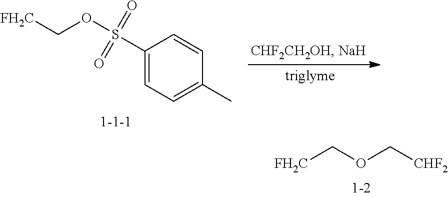

TABLE S14-1

| | | Added | | |
|---|---|---|---|---|
| Reactant | M.W. | amount | | Equiv. |
| 1-1-1 | 218.21 | 800 | g | 1.0 |
| 2-fluoroethanol | 82.04 | 391 | g | 1.3 |
| NaH (60% in oil) | 24 | 190 | g | 1.3 |
| Triglyme (solvent) | | 3.5 | L | |

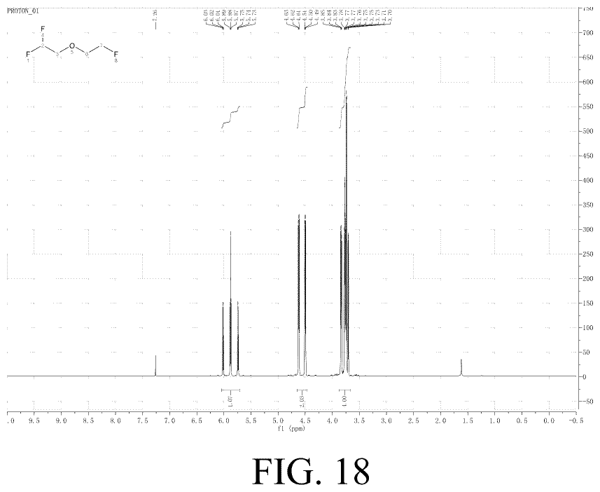

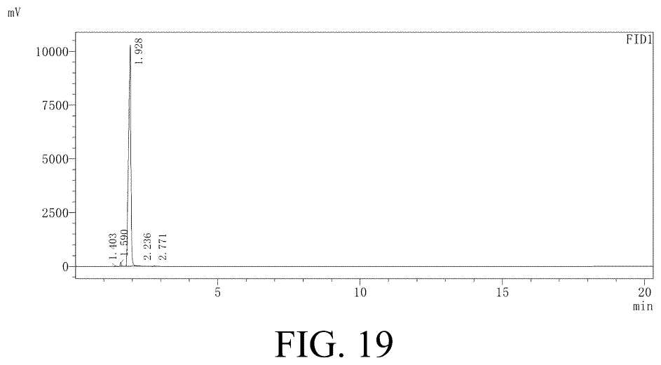

Under nitrogen atmosphere, in a reactor were added NaH and triglyme. While stirring at room temperature, 2,2-difluoroethanol was added slowly into the suspension. After the addition of 2,2-difluoroethanol, Intermediate 1-1-1 was added slowly into the suspension. The suspension was warmed up and allowed to react overnight. After the reaction, the suspension was cooled down to 0° C. again and cold saturated NaCl solution was added to quench excess NaH. DCM was used to extract the system. The organic layer was washed by saturated NaCl solution and dried with Na₂SO₄. Rotary evaporation was used to obtain concentrated crude product. The crude product underwent vacuum distillation to obtain a colorless liquid as the final product with a purity of 99.8%. Final yield after distillation was ˜85%. ¹H-NMR and GC-MS for the resulting product (Compound 2) are shown in FIG. 18 and FIG. 19, respectively.

### Example 15: Synthesis of 1-fluoro-3-(3-fluoropropoxy)propane (Compound 10)

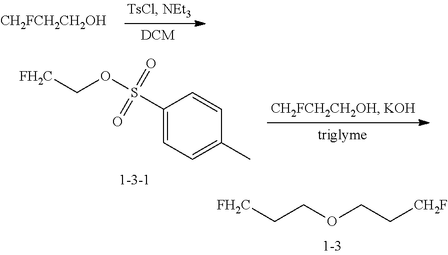

Step 1: Synthesis of 3-fluoropropyl 4-methylbenzenesulfonate (Intermediate 1-3-1)

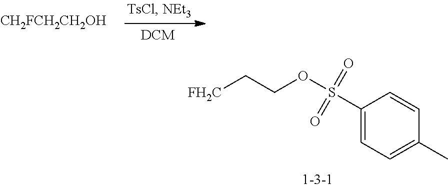

TABLE S15-1

| | | Added | | |
|---|---|---|---|---|
| Reactant | M.W. | amount | | Equiv. |
| 3-fluoropropanol | 78.09 | 65 | g | 1.0 |
| TsCl | 190.65 | 165.86 | g | 1.05 |
| triethylamine | 101.19 | 126.48 | g | 1.5 |
| DCM (solvent) | | 0.5 | L | |

In a reactor were added 3-fluoropropanol, triethylamine and DCM, and the mixed solution was stirred at 0° C. for 30 min. TsCl was added in batches and the system was stirred at room temperature for 6 hours. After the reaction, NH₄Cl aqueous solution was used to wash the DCM layer to remove excess triethyl amine. The organic layer was further washed with saturated NaCl solution and dried with Na₂SO₄. Rotary evaporation was used to obtain a red oil as the crude product, which was used in the next step without strict purification. Yield was ˜95%.

### Step 2: Synthesis of 1-fluoro-3-(3-fluoropropoxy)propane (Compound 10)

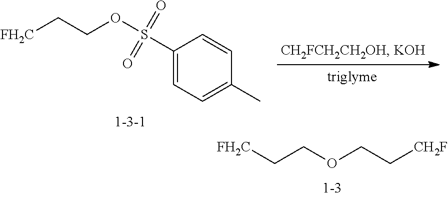

TABLE S15-2

| | | Added | | |
|---|---|---|---|---|
| Reactant | M.W. | amount | | Equiv. |
| 1-3-1 | 232.21 | 168 | g | 1.0 |
| 3-fluoropropanol | 78.09 | 73.3 | g | 1.3 |
| KOH | 56.11 | 60.6 | g | 1.5 |
| Triglyme (solvent) | | 0.9 | L | |

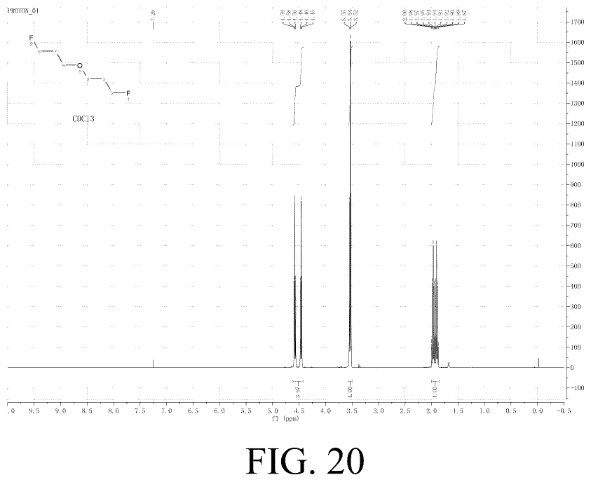

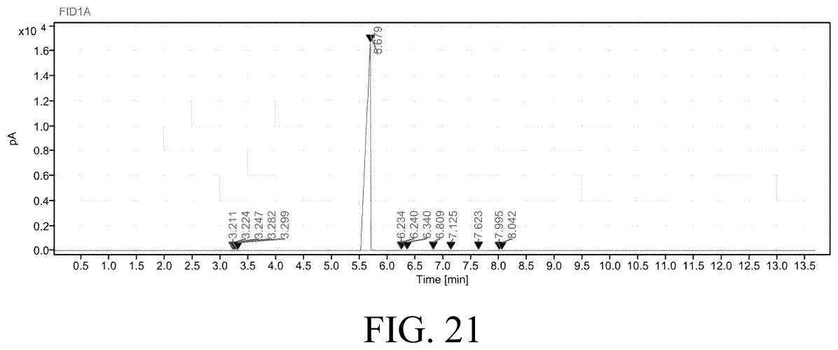

Under nitrogen atmosphere, in a reactor were added KOH and triglyme. While stirring at room temperature, 3-fluoropropanol was added slowly into the suspension. After the addition of 3-fluoropropanol, Intermediate 1-3-1 was added slowly into the suspension. The suspension was warmed up and allowed to react overnight. DCM was used to extract the system. The organic layer was washed by saturated NaCl solution and dried with Na₂SO₄. Rotary evaporation was used to obtain concentrated crude product. The crude product underwent vacuum distillation to obtain a colorless liquid as the final product with a purity of 99.5%. Final yield after distillation was ˜76%. ¹H-NMR and GC-MS for the resulting product (Compound 10) are shown in FIG. 20 and FIG. 21, respectively.

### Example 16: Synthesis of tris(2-fluoroethoxy)methane (Compound 23)

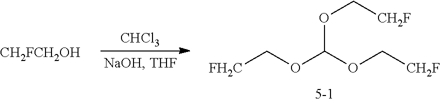

TABLE S16-1

| | | Added | | |
|---|---|---|---|---|
| Reactant | M.W. | amount | | Equiv. |
| 2-fluoroethanol | 64.06 | 700 | g | 3.0 |
| CHCl₃ | 119.38 | 434 | g | 1.0 |
| NaOH | 40 | 437 | g | 3.0 |
| THF (solvent) | | 3.5 | L | |

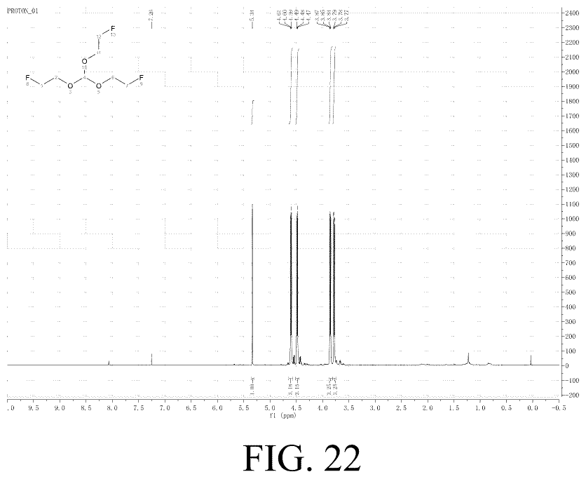

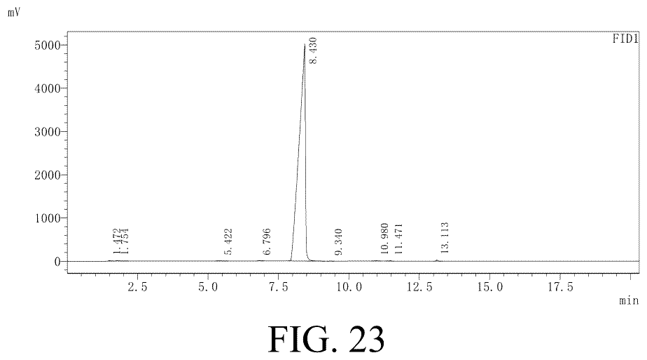

Under nitrogen atmosphere, in a reactor were added 2-fluoroethanol, NaOH and THF. The suspension was stirred at room temperature. While stirring, CHCl₃ was added slowly into the suspension and then the suspension was stirred for overnight. After the reaction, the suspension was washed with water and extracted with methyl t-butyl ether for several times. The organic layer was washed by saturated NaCl solution and dried with Na₂SO₄. Rotary evaporation was used to obtain concentrated crude product. The crude product underwent vacuum distillation to obtain a colorless liquid as the final product with a purity of 99.5%. Final yield after distillation was ˜50%. ¹H-NMR and GC-MS for the resulting product (Compound 23) are shown in FIG. 22 and FIG. 23, respectively.

### Example 17: Synthesis of 1,1-Bis(2-fluoroethoxy)methane

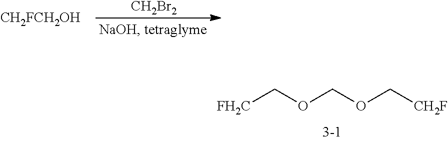

TABLE S17-1

| | | Added | | |
|---|---|---|---|---|
| Reactant | M.W. | amount | | Equiv. |
| 2-fluoroethanol | 64.06 | 1900 | g | 2.1 |
| Dibromomethane | 173.84 | 2454 | g | 1.0 |
| NaOH | 40 | 1186 | g | 2.1 |
| Tetraglyme (solvent) | | 9.5 | L | |

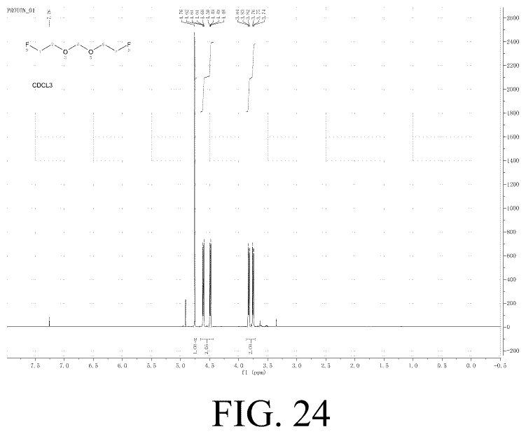

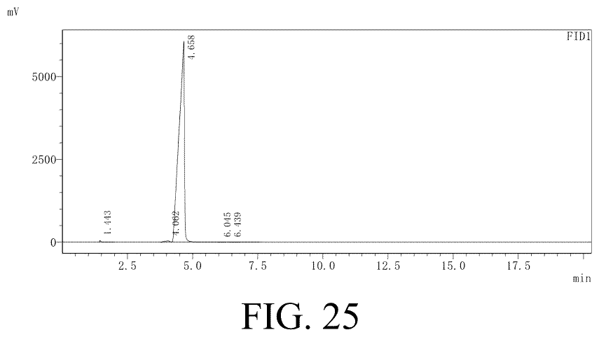

Under nitrogen atmosphere, in a reactor were added NaOH and tetraglyme. The suspension was stirred at room temperature. While stirring, dibromomethane was added slowly into the suspension and then the suspension was warmed up to react for 2 hours. Then 2-fluoroethanol was added slowly into the suspension and the mixture was further warmed up to reflux overnight. After the reaction, the suspension was washed with water and extracted with hexane several times. The organic layer was washed by saturated NaCl solution and dried with Na₂SO₄. Rotary evaporation was used to obtain concentrated crude product. The crude product underwent vacuum distillation to obtain a colorless liquid as the final product with a purity of 99.5%. Final yield after distillation was ˜35%. ¹H-NMR and GC-MS for the resulting product (1,1-Bis(2-fluoroethoxy)methane) are shown in FIG. 24 and FIG. 25, respectively.

### Example 18: Synthesis of 1,1-Bis(2,2-difluoroethoxy)methane

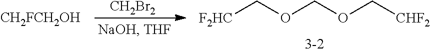 [SMILES: "CH2FCH2OH" ; "CH2Br2" ; FC(F)COCOCC(F)F]

TABLE S18-1

| | | Added | | |
|---|---|---|---|---|
| Reactant | M.W. | amount | | Equiv. |
| 2,2-difluoroethanol | 82.04 | 2400 | g | 2.0 |
| Dibromomethane | 173.84 | 2542 | g | 1.0 |
| NaOH | 40 | 1170 | g | 2.0 |
| THF (solvent) | | 12 | L | |

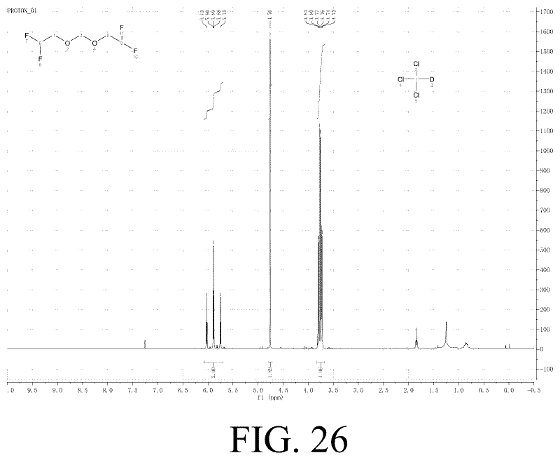

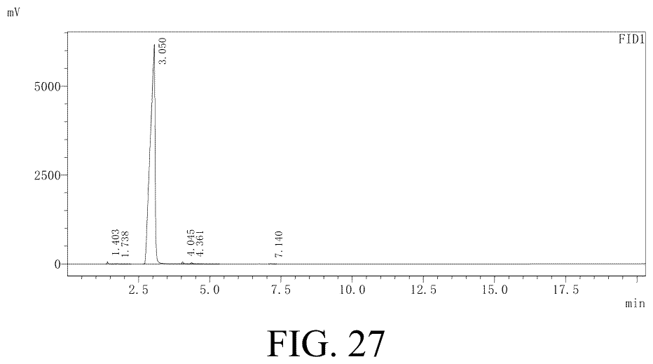

Under nitrogen atmosphere, in a reactor were added NaOH and THF. The suspension was stirred at room temperature. While stirring, dibromomethane was added slowly into the suspension and then the suspension was warmed up to react for 2 hours. Then 2,2-difluoroethanol was added slowly into the suspension and the mixture was further warmed up to reflux overnight. After the reaction, the suspension was washed with water and extracted with hexane several times. The organic layer was washed by saturated NaCl solution and dried with Na₂SO₄. Rotary evaporation was used to obtain concentrated crude product. The crude product underwent vacuum distillation to obtain a colorless liquid as the final product with a purity of 99.5%. Final yield after distillation was ˜60%. ¹H-NMR and GC-MS for the resulting product (1,1-Bis(2,2-difluoroethoxy)methane) are shown in FIG. 26 and FIG. 27, respectively.

The invention claimed is:

1: A compound of Formula (I), Formula (II), or Formula (III): 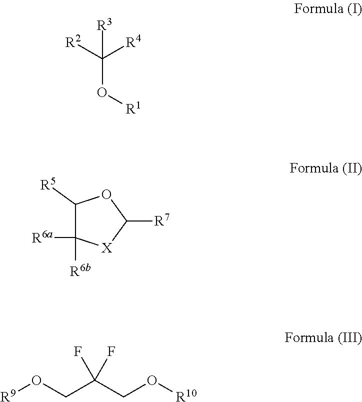
wherein:
X is selected from the group consisting of *—(CH₂)ₙO—**, *—(CH₂)ₙ—**, and *—O(CHR⁸)—**, wherein * denotes the point of attachment to the carbon bearing R^{6a} and R^{6b}, and ** denotes the point of attachment to the carbon bearing R⁷;
n is 0-3;
R¹ is C₁-C₄ alkyl substituted by one or more halogen, wherein each halogen is independently selected;
R² is selected from the group consisting of R^{2′} and —OR^{2′}, wherein R^{2′} is C₁-C₄ alkyl substituted by one or more halogen, wherein each halogen is independently selected;
R³ is selected from the group consisting of H, R^{3′}, and —OR^{3′}, wherein R^{3′} is C₁-C₄ alkyl substituted by one or more halogen, wherein each halogen is independently selected;
R⁴ is selected from the group consisting of H, R^{4′}, and —OR^{4′}, wherein R^{4′} is C₁-C₄ alkyl substituted by one or more halogen, wherein each halogen is independently selected;
R⁵ is selected from the group consisting of H, R^{5′}, and —CH₂OR^{5′}, wherein R^{5′} is C₁-C₄ alkyl substituted by one or more halogen, wherein each halogen is independently selected;
R^{6a} is selected from the group consisting of H, halogen, R^{6′}, and —CH₂OR^{6′}, wherein R^{6′} is C₁-C₄ alkyl substituted by one or more halogen, wherein each halogen is independently selected;
R^{6b} is selected from the group consisting of H and halogen;
R⁷ is selected from the group consisting of H, R^{7′}, and —CH₂OR^{7′}, wherein R^{7′} is C₁-C₄ alkyl substituted by one or more halogen, wherein each halogen is independently selected; and
R⁸ is selected from the group consisting of H, R^{8′}, and —CH₂OR^{8′}, wherein R^{8′} is C₁-C₄ alkyl substituted by one or more halogen, wherein each halogen is independently selected;
R⁹ is C₁-C₄ alkyl;
R¹⁰ is C₁-C₄ alkyl;
provided that when R⁷ is H, R^{6a} is H, and R^{6b} is H, then R⁵ is R^{5′}, and
provided that the compound is not

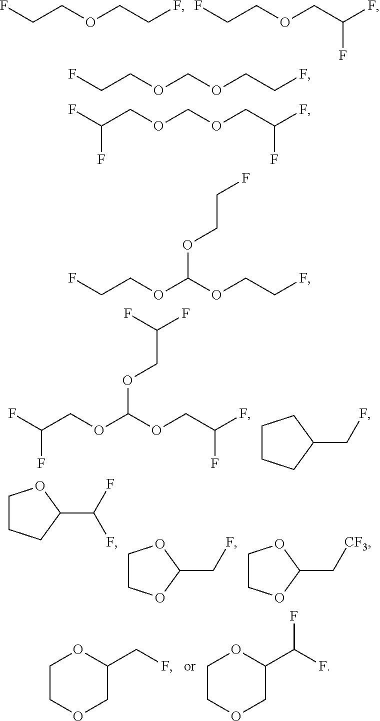

2: The compound of claim 1, wherein the compound is a compound of Formula (I).

3: The compound of claim 1, wherein the compound is a compound of Formula (II).

4: The compound of claim 1, wherein the compound is a compound of Formula (III).

5: The compound of claim 1, wherein the compound is a compound of Formula (I), and wherein
R² is R^{2′};
R³ is R^{3′}; and
R⁴ is R^{4′}.

6-7. (canceled)

8: The compound of claim 1, wherein the compound is a compound of Formula (I), and wherein
R² is —OR^{2′};
R³ is H; and
R⁴ is H.

9-11. (canceled)

12: The compound of claim 1, wherein the compound is a compound of Formula (I), and wherein
R² is —OR^{2′};
R³ is —OR^{3′}; and
R⁴ is H.

13-14. (canceled)

15: The compound of claim 1, wherein the compound is a compound of Formula (I), and wherein
R² is —OR^{2′};
R³ is —OR^{3′}; and
R⁴ is —OR^{4′}.

16. (canceled)

17: The compound of claim 1, wherein the compound is a compound of Formula (II), and wherein X is *—(CH₂)ₙO—**, wherein * denotes the point of attachment to the carbon bearing R^{6a} and R^{6b}, and ** denotes the point of attachment to the carbon bearing R⁷.

18-21. (canceled)

22: The compound of claim 1, wherein the compound is a compound of Formula (II), and wherein X is *—(CH₂)ₙ—**, wherein * denotes the point of attachment to the carbon bearing R^{6a} and R^{6b}, and ** denotes the point of attachment to the carbon bearing R⁷.

23-26. (canceled)

27: The compound of claim 1, wherein the compound is a compound of Formula (II), and wherein X is *—O(CHR⁸)—**, wherein * denotes the point of attachment to the carbon bearing R^{6a} and R^{6b}, and ** denotes the point of attachment to the carbon bearing R⁷.

28-29. (canceled)

30: A compound selected from the group consisting of:

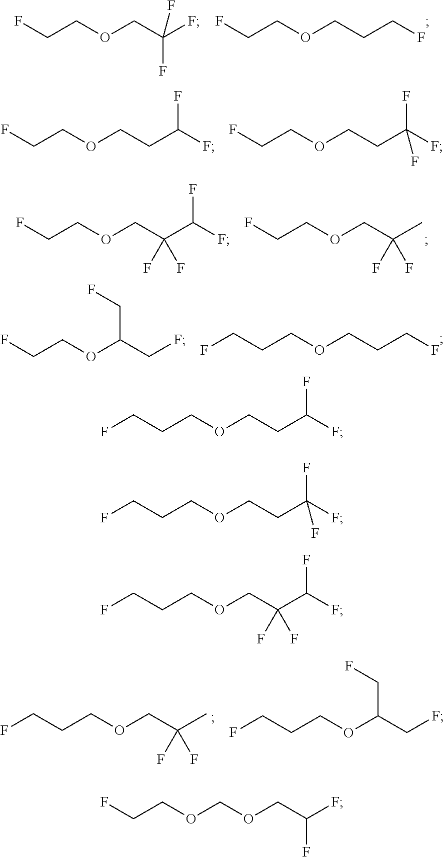

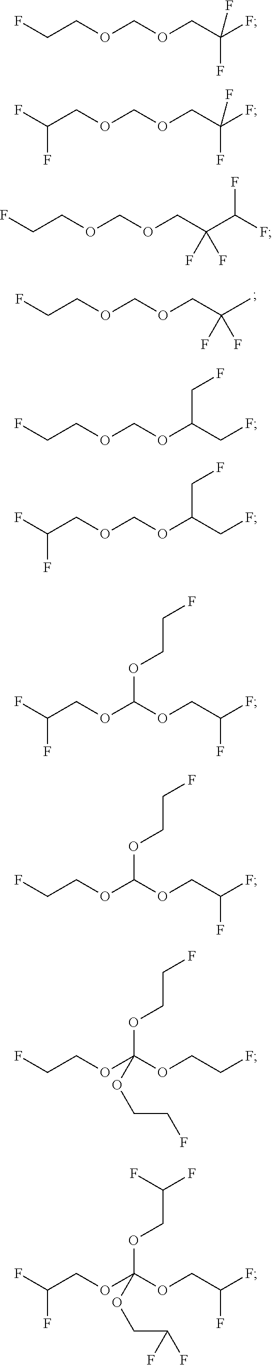

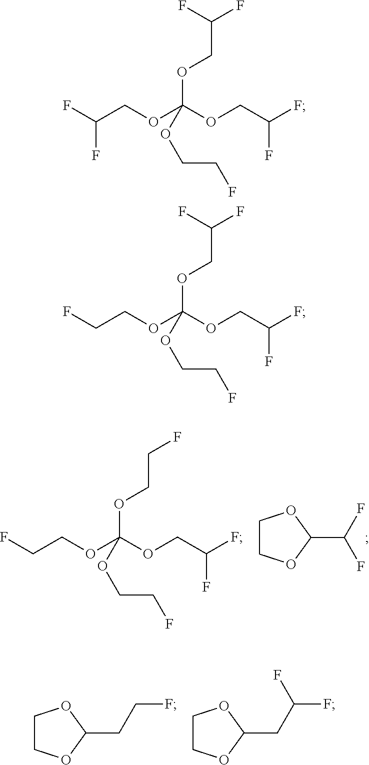

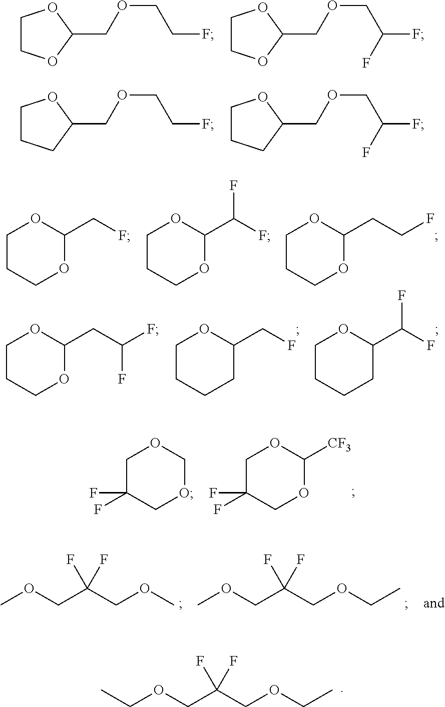

31: An electrolyte comprising a first halogenated ether component of Formula (I), Formula (II), or Formula (III): 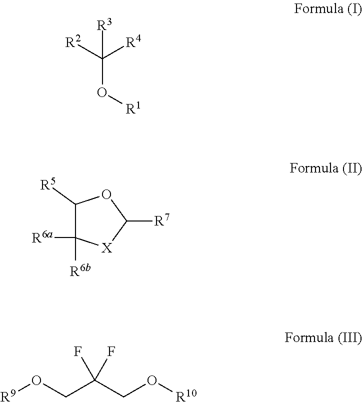
wherein:
X is selected from the group consisting of *—(CH₂)ₙO—**, *—(CH₂)ₙ—**, and *—O(CHR⁸)—**, wherein * denotes the point of attachment to the carbon bearing R^{6a} and R^{6b}, and ** denotes the point of attachment to the carbon bearing R⁷;
n is 0-3;
R¹ is C₁-C₄ alkyl substituted by one or more halogen, wherein each halogen is independently selected;
R² is selected from the group consisting of R^{2′} and —OR^{2′}, wherein R^{2′} is C₁-C₄ alkyl substituted by one or more halogen, wherein each halogen is independently selected;
R³ is selected from the group consisting of H, R^{3′}, and —OR^{3′}, wherein R^{3′} is C₁-C₄ alkyl substituted by one or more halogen, wherein each halogen is independently selected;
R⁴ is selected from the group consisting of H, R^{4′}, and —OR^{4′}, wherein R^{4′} is C₁-C₄ alkyl substituted by one or more halogen, wherein each halogen is independently selected;
R⁵ is selected from the group consisting of H, R^{5′}, and —CH₂OR^{5′}, wherein R^{5′} is C₁-C₄ alkyl substituted by one or more halogen, wherein each halogen is independently selected;
R^{6a} is selected from the group consisting of H, halogen, R^{6′}, and —CH₂OR^{6′}, wherein R^{6′} is C₁-C₄ alkyl substituted by one or more halogen, wherein each halogen is independently selected;
R^{6b} is selected from the group consisting of H and halogen;
R⁷ is selected from the group consisting of H, R^{7′}, and —CH₂OR^{7′}, wherein R^{7′} is C₁-C₄ alkyl substituted by one or more halogen, wherein each halogen is independently selected;
R⁸ is selected from the group consisting of H, R^{8′}, and —CH₂OR^{8′}, wherein R^{8′} is C₁-C₄ alkyl substituted by one or more halogen, wherein each halogen is independently selected; and
R⁹ is C₁-C₄ alkyl;
R¹⁰ is C₁-C₄ alkyl,
provided that when R⁷ is H, R^{6a} is H, and R^{6b} is H, then R⁵ is R^{5′}, and
provided that the compound is not

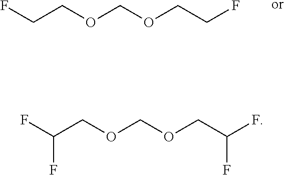 [SMILES: FCCOCOCCF ; "or" ; FC(F)COCOCC(F)F]

32-50. (canceled)

51: The electrolyte of claim 31, wherein the first halogenated ether component is a compound selected from the group consisting of:

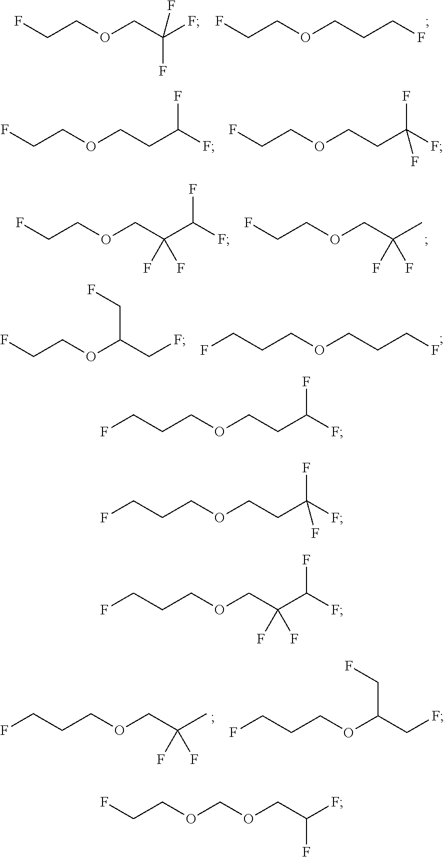

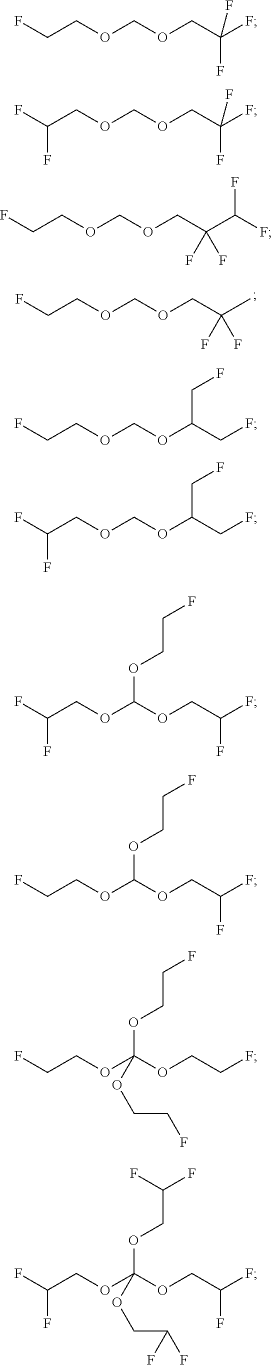

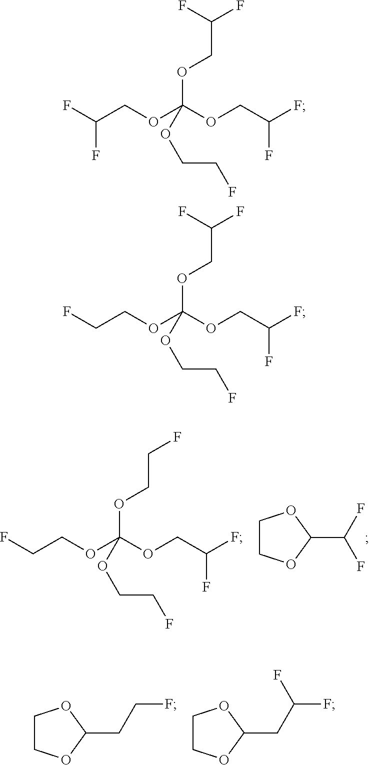

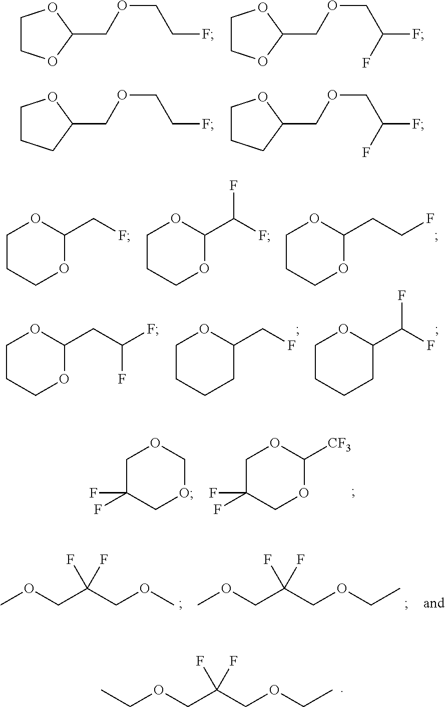

52: The electrolyte of claim 31, wherein the electrolyte comprises one or more additional halogenated ether components, each of which is independently a compound of Formula (I), Formula (II), or Formula (III).

53: The electrolyte of claim 31, wherein the electrolyte comprises a solvent component that is not a compound of Formula (I), Formula (II), or Formula (III).

54: The electrolyte of claim 53, wherein the solvent component is selected from the group consisting of ethylene carbonate (EC); propylene carbonate (PC); dimethyl carbonate (DMC); diethyl carbonate (DEC); ethyl methyl carbonate (EMC); vinyl carbonate (VC); vinyl ethylene carbonate (VEC); fluoroethylene carbonate (FEC); difluoroethylene carbonate (DFEC); 3,3,3-trifluoropropylene carbonate (TFPC); monofluoroethyl methyl carbonate (F1EMC); difluoroethyl methyl carbonate (F2EMC); trifluoroethyl methyl carbonate (F3EMC); bis(2,2,2-trifluoroethyl) carbonate (TFEC); succinic anhydride (SA), butyric anhydride (BA); 1,2-dimethyoxylethane (DME); 1,3-dioxolane (DOL); 1,4-dioxane (DOX); tetrahydrofuran (THF); tetravinyl silane (TVSI); acetonitrile (AN); ethyl acetate (EA); methyl acetate (MA); methyl propanoate (MP); succinonitrile (SN); adiponitrile (ADN); 1,3,6-Hexanetricarbonitrile (HTCN); trimethyl borate (TMB); triphenyl borate (TPB); triethyl borate (TEB); tris(pentafluorophenyl)borane (TPFPB); tris(trimethylsilyl)phosphate (TTSB); tris(2,2,2-trifluoroethyl) borate (TTFEB); trimethyl phosphate (TMP); triethyl phosphate (TEP); tris(trimethylsilyl)phosphate (TTSP); tris(trimethylsilyl)phosphite (TTSPi); tris(2,2,2-trifluoroethyl) phosphate (TFEPa); tris(2,2,2-trifluoroethyl) phosphite (TFEPi); (pentafluorophenyl)diphenyl phosphine (PFPDPP); tris(pentafluorophenyl) phosphine (TPFPP); 1,3,2-dioxathiolane-2,2-dioxide (DTD); 1,3-propanesultone (PS); prop-1-ene-1,3-sultone (PES); propanediol cyclic sulfate (PCS); ethylene sulfite (ES); 1,4-butane sultone (BS); dimethyl sulfoxide (DMSO); methylene methanedisulfonate (MMDS); N,N-Dimethylformamide (DMF); gamma-butyrolactone (BL); bis(2,2,2-trifluoroethyl) ether (BTFE); 1,1,2,2-tetrafluoroethyl-2,2,3,3-tetrafluoropropyl ether (TTE); 1H,1H,5H-octafluoropentyl-1,1,2,2-tetrafluoroethylether (OTE); 2,2,2-trifluoroethyl 1,1,2,2-tetrafluoroethyl ether (HFE); tris(2,2,2-trifluoroethyl) orthoformate (TFEO); 1,2-bis(1,1,2,2-tetrafluoroethoxy)ethane (F8DEE); 1,1,1,2,3,3-hexafluoro-3-(2,2,2-trifluoroethoxy)propane; hexafluoroisopropyl methyl ether; 1-fluoro-2-(2-methoxyethoxy)ethane; 1,1-difluoro-2-(2-methoxyethoxy)ethane; 1,1,1-trifluoro-2-(2-methoxyethoxy)ethane; 1-ethoxy-2-(2-fluoroethoxy)ethane; 2-(2-ethoxyethoxy)-1,1-difluoroethane; 1,2-bis(2-fluoroethoxy)ethane; 1,1-difluoro-2-(2-(2-fluoroethoxy)ethoxy)ethane; 1,1,1-trifluoro-2-(2-(2-fluoroethoxy)ethoxy)ethane; 2-(2-ethoxyethoxy)-1,1,1-trifluoroethane; 1,2-bis(2,2-difluoroethoxy)ethane; 2-(2-(2,2-difluoroethoxy)ethoxy)-1,1,1-trifluoroethane; or 1,2-bis(2,2,2-trifluoroethoxy)ethane; 2-fluoro-1,3-dimethoxypropane; 2-fluoro-1,3-diethoxypropane; 2,2-difluoro-1,3-dimethoxypropane; or 2,2-difluoro-1,3-diethoxypropane; 2-fluoro-1,3-bis(2-fluoroethoxy)propane; 2-fluoro-1,3-bis(2,2-difluoroethoxy)propane; 2-fluoro-1,3-bis(2,2,2-trifluoroethoxy)propane; 2,2-difluoro-1,3-bis(2-fluoroethoxy)propane; 2,2-difluoro-1,3-bis(2,2-difluoroethoxy)propane; 2,2-difluoro-1,3-bis(2,2,2-trifluoroethoxy)propane; and mixtures of any of the foregoing.

55: The electrolyte of claim 53, wherein the amount of the solvent component in the electrolyte is between about 0.5 wt. % and about 99 wt. %.

56: The electrolyte of claim 31, wherein the electrolyte comprises one or more salts.

57: The electrolyte of claim 56, wherein the salt is selected from the group consisting of a lithium salt; a potassium salt; a sodium salt; a cesium salt; a magnesium salt; a zinc salt; a calcium salt; a silver salt; an aluminum salt; a lanthanum salt, and mixtures of any of the foregoing.

58: The electrolyte of claim 57, wherein the salt is selected from the group consisting of lithium bis(fluorosulfonyl)imide (LiFSI); lithium bis(trifluoromethanesulfonyl)imide (LiTFSI); lithium bis(pentafluoroethanesulfonyl)imide (LiBETI); lithium hexafluorophosphate (LiPF₆); lithium hexafluoroarsenate (LiAsF₆); lithium tetrafluoroborate (LiBF₄); lithium bis(oxalato)borate (LiBOB); lithium difluoro(oxalato)borate (LiDFOB); lithium difluorophosphate (LiDFP); lithium difluoro(dioxalato)phosphate (LiDFDOP); lithium tetrafluoro(oxalato)phosphate (LiTFOP); lithium nitrate (LiNO₃); lithium perchlorate (LiClO₄); lithium triflate (LiTf); lithium trifluoroacetate (LiTFA); lithium 4,5-dicyano-2-(trifluoromethyl)imidazole (LiTDI); sodium hexafluorophosphate (NaPF₆); sodium bis(fluorosulfonyl)imide (NaFSI); sodium bis(trifluoromethanesulfonyl)imide (NaTFSI); sodium triflate (NaTf); potassium hexafluorophosphate (KPF₆); potassium bis(fluorosulfonyl)imide (KFSI); potassium bis(trifluoromethanesulfonyl)imide (KTFSI); potassium triflate (KTf); cesium bis(fluorosulfonyl)imide (CsFSI); cesium bis(trifluoromethanesulfonyl)imide (CsTFSI); magnesium bis(trifluoromethanesulfonyl)imide (Mg(TFSI)₂); zinc bis(trifluoromethanesulfonyl)imide (Zn(TFSI)₂); calcium bis(trifluoromethanesulfonyl)imide (Ca(TFSI)₂); silver bis(trifluoromethanesulfonyl)imide (AgTFSI); aluminum bis(trifluoromethanesulfonyl)imide (Al(TFSI)₃); lanthanum bis(trifluoromethanesulfonyl)imide (La(TFSI)₃), and mixtures of any of the foregoing.

59: An electrochemical cell comprising:
an anode;
a cathode; and
the electrolyte of claim 31.

60: The electrochemical cell of claim 59, wherein the electrochemical cell is a battery.

61: The electrochemical cell of claim 59, wherein the anode comprises an element selected from the group consisting of lithium, sodium, and potassium.

62: The electrochemical cell of claim 59, wherein the anode comprises lithium metal, sodium metal, lithium-magnesium alloy, or lithium-aluminum alloy.

63: The electrochemical cell of claim 59, wherein the anode comprises a surface protection layer comprising fluorine.

64: The electrochemical cell of claim 59, wherein the anode comprises a material selected from the group consisting of lithium metal, graphite, silicon, silicon oxide (SiOₓ), graphite/silicon composite, graphite/silicon oxide (SiOₓ) composite, graphite/silicon nitride (Si₃N₄) composite, graphite/silicon carbide (SiC) composite, sodium metal, hard carbon, potassium metal, and mixtures of any of the foregoing.

65: The electrochemical cell of claim 59, wherein the cathode comprises sulfur, a lithium nickel manganese cobalt oxide, a lithium nickel cobalt aluminum oxide (NCA), a lithium nickel manganese aluminum oxide (NMA), a lithium nickel manganese cobalt aluminum oxide (NMCA), a lithium nickel oxide (LNO), a lithium nickel manganese oxide (LiNi_{0.5}Mn_{1.5}O₄), a lithium cobalt oxide (LCO), a lithium manganese oxide (LMO), a lithium and manganese rich cathode (LMR or LLMO), a lithium iron phosphate (LFP), a lithium cobalt phosphate (LCP), a lithium manganese phosphate (LMP), a lithium manganese iron phosphate (LMFP), a transition metal sulfide, a sodium vanadium phosphate (Na₃V₂(PO₄)₃), a sodium copper nickel iron manganese oxide (Na[Cu_{1/9}Ni_{2/9}Fe_{1/3}Mn_{1/3}]O₂), a Prussian white (R—Na_{1.92}Fe[Fe(CN)₆]), and mixtures of any of the foregoing.

66: A method of preparing the compound of claim 5, comprising reacting a compound of Formula (S1) 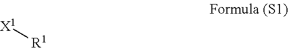
with a compound of Formula (S2) 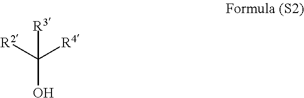
and a base, to provide the compound of claim 5, wherein X¹ is selected from the group consisting of 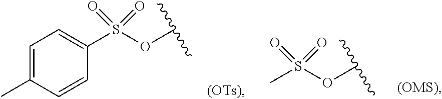
Cl, Br, and I.

67: A method of preparing the compound of claim 8, comprising reacting a compound of Formula (S3) 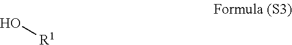
and a compound of formula (S4) 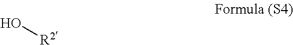
with a compound of 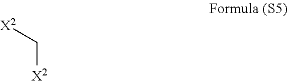
and a base, to provide the compound of claim 8, wherein each X² is independently selected from the group consisting of Cl, Br, and I.

68: A method of preparing the compound of claim 12, comprising reacting a compound of Formula (S3) 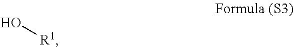
a compound of Formula (S4) 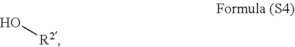
a compound of Formula (S6) 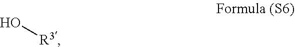
with a compound of Formula (S7) 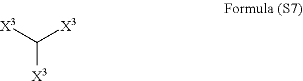
and a base, to provide the compound of claim 12, wherein each X³ is independently selected from the group consisting of Cl, Br, and I.

69: A method of preparing the compound of claim 15, comprising reacting a compound of Formula (S3) 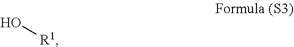
a compound of Formula (S4) 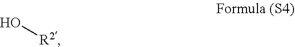
a compound of Formula (S6) 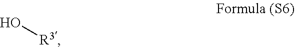
a compound of Formula (S8) 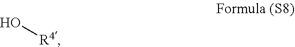
with a compound of Formula (S9) 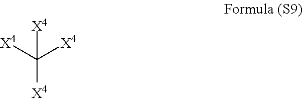
and a base, to provide the compound of claim 15, wherein each X⁴ is independently selected from the group consisting of Cl, Br, and I.

70: A method of preparing the compound of claim 4, comprising reducing a compound of Formula (S10) 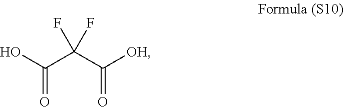
to provide a compound of Formula (S11) 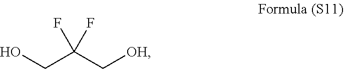
and reacting the compound of Formula (S11) with a compound of Formula (S12) 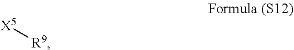
a compound of Formula (S13) 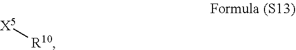
to provide the compound of claim 4, wherein each X⁵ is independently selected form the group consisting of 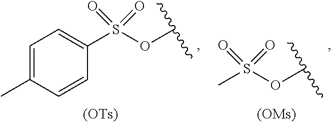
Cl, Br, and I.
